(19) United States
(12) Patent Application Publication
(10) Pub. No.: US 2006/0024684 A1
(43) Pub. Date: Feb. 2, 2006

(54) Prognostic markers for prediction of treatment response and/or survival of breast cell proliferative disorder patients

(75) Inventors: John Foekens, Rotterdam (NL); Nadia Harbeck, Otterfing (DE); Thomas Koenig, Berlin (DE); Sabine Maier, Berlin (DE); John Martens, Rotterdam (NL); Fabian Model, Berlin (DE); Inko Nimmrich, Berlin (DE); Tamas Rujan, Berlin (DE); Manfred Schmitt, Munich (DE); Ralf Lesche, Berlin (DE); Dimo Dietrich, Berlin (DE); Volkmar Mueller, Hamburg (DE); Antje Kluth, Berlin (DE); Ina Schwope, Berlin (DE); Oliver Hartmann, Berlin (DE); Peter Adorjan, Berlin (DE)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Epigenomics AG

(21) Appl. No.: 11/011,332

(22) Filed: Dec. 13, 2004

Related U.S. Application Data
(63) Continuation-in-part of application No. 10/517,741.

(30) Foreign Application Priority Data
Oct. 1, 2002 (DE) 10245779.4
Jan. 7, 2003 (DE) 10300096.8
Apr. 17, 2003 (DE) 10317955.0
Dec. 13, 2004 (WO) PCT/EP04/14170
Dec. 11, 2003 (EP) 03090432.0
Feb. 10, 2004 (EP) 04090041.7
Sep. 30, 2004 (EP) 04090380.9
Apr. 1, 2004 (EP) 04090127.4
Nov. 16, 2004 (EP) 04027213.0
Jun. 5, 2004 (EP) 04013328.2

(51) Int. Cl. C12Q 1/68

(52) U.S. Cl. 435/6

(57) ABSTRACT

Aspects of the present invention provide compositions and methods for prognosis of, and/or predicting the estrogen treatment outcome of breast cell proliferative disorder patients, and in particular of patients with breast carcinoma. In preferred embodiments, this is achieved, at least in part, by determining the expression level of PITX2, and/or the genetic or the epigenetic modifications of the genomic DNA associated with the gene PITX2. Additional aspects of the invention provide novel sequences, oligomers (e.g., oligonucleotides or peptide nucleic acid (PNA)-oligomers), and antibodies, which have substantial utility in the described inventive methods and compositions.

### CROSS-REFERENCE TO RELATED APPLICATIONS

This application is a continuation-in-part of U.S. application Ser. No. ______, filed on 11 Dec. 2004 and entitled METHOD AND NUCLEIC ACIDS FOR THE IMPROVED TREATMENT OF BREAST PROLIFERATIE DISORDERS, and additionally claims the additional benefit of priority to: PCT/EP03/010881 of same title, international filing date 1 Oct. 2003; DE 10245779.4, filed 1 Oct. 2002; DE 10300096.8, filed 7 Jan. 2003; DE 10317955.0, filed 17 Apr. 2003; PCT/______, international filing date 13 Dec. 2004, of same title; EP 03 090 432.0, filed 11 Dec. 2003; EP 04 090 041.7, filed 10 Feb. 2004; EP 04 090 380.9, filed 30 Sep. 2004; EP 04 090 127.4, filed 1 Apr. 2004; EP 04 027 213.0, filed 16 Nov. 2004; and EP 04 013 328.2, filed 5 Jun. 2004; all of which are incorporated by reference herein in their entireties.

### FIELD OF THE INVENTION

The present invention relates generally to methods having utility for predicting the survival and/or treatment response of a patient diagnosed with a cell proliferative disorder of the breast tissues, and in particular aspects, to determining PITX2 gene expression level and/or determining the genetic and/or the epigenetic modifications of the genomic DNA associated with the gene PITX2 and/or the regulatory or promoter regions thereof. Additional aspects relate to novel nucleic acids, oligonucleotides, antibodies, and compositions thereof, having utility in the described methods.

### SEQUENCE LISTING

A Sequence Listing, pursuant to 37 C.F.R. § 1.52(e)(5), has been provided on compact disc (1 of 1) as a 0.735 MB text file, entitled “47675-99 Sequence Listing.txt,” and which is incorporated by reference herein in its entirety.

### BACKGROUND

BREAST CANCER. Breast cancer is the most frequently diagnosed cancer and the second leading cause of cancer death in European and American women. In women aged 40-55, breast cancer is the leading cause of death (Greenlee et al., 2000). In 2002 there were 204,000 new cases of breast cancer in the US, and a comparable number in Europe.

Breast cancer is defined as the uncontrolled proliferation of cells within breast tissues. Breasts are comprised of 15 to 20 lobes joined together by ducts. Cancer arises most commonly in the duct, but is also found in the lobes, with the rarest type of cancer termed inflammatory breast cancer. It will be appreciated by those skilled in the art that there exists a continuing need to improve methods of early detection, classification and treatment of breast cancers. In contrast to the detection of some other common cancers such as cervical and dermal there are inherent difficulties in classifying and detecting breast cancers.

BREAST CANCER TREATMENT. The first step of any treatment is the assessment of the patient's condition comparative to defined classifications of the disease. However the value of such a system is inherently dependent upon the quality of the classification. Breast cancers are staged according to their size, location and occurrence of metastasis. Methods of treatment include the use of surgery, as well as radiation therapy, chemotherapy and endocrine therapy, which are also used as adjuvant therapies to surgery. Generally, more aggressive diseases are regarded as requiring treatment with more aggressive therapies.

Although the vast majority of early cancers are operable, (i.e., the tumor can be completely removed by surgery), about one third of the patients with lymph-node negative diseases and about 50-60% of patients with node-positive disease will develop metastases during follow-up.

Based on this observation, systemic adjuvant treatment has been introduced for both node-positive and node-negative breast cancers. Systemic adjuvant therapy is administered after surgical removal of the tumor, and has been shown to reduce the risk of recurrence significantly. Several types of adjuvant treatment are available: endocrine treatment, also called hormone treatment (for hormone receptor positive tumors); different chemotherapy regimens; and antibody treatments, based on novel agents like Herceptin (an antibody to an epidermal growth factor receptor).

The growth of the majority (app. 70-80%) of breast cancers is dependent on the presence of estrogen. Therefore, one important target for adjuvant therapy is the removal of estrogen (e.g, by ovarian ablation), the blocking of its synthesis or the blocking of its actions on the tumor cells, either by blocking the receptor with competing substances (e.g., Tamoxifen) or by inhibiting the conversion of androgen into estrogen (e.g., aromatase inhibitors). This type of treatment is referred to in the art as “endocrine treatment.” Endocrine treatment is thought to be efficient only in tumors that express hormone receptors (the estrogen receptor (ER), and/or the progesterone receptor (PR)). Currently, the vast majority of women with hormone receptor positive breast cancer receive some form of endocrine treatment, independent of their nodal status. The most frequently used drug in this scenario is Tamoxifen.

However, even in hormone receptor positive patients, not all patients benefit from endocrine treatment. Adjuvant endocrine therapy reduces mortality rates by 22% while response rates to endocrine treatment in the metastatic (advanced) setting are 50 to 60%.

Because Tamoxifen has relatively few side effects, treatment may be justified even for patients with low likelihood of benefit. However, these patients may require additional, more aggressive adjuvant treatment. Even in earliest and least aggressive tumors, such as node-negative, hormone receptor positive tumors, about 21% of patients relapse within 10 years after initial diagnosis if they receive Tamoxifen monotherapy as the only adjuvant treatment (Lancet. 351:1451-67, 1998; Tamoxifen for early breast cancer: an overview of the randomized trials; Early Breast Cancer Trialists' Collaborative Group). Similarly, some patients with hormone receptor negative disease may be treated sufficiently with surgery and potentially radiotherapy alone, whereas others may require additional chemotherapy.

Several cytotoxic regimens have shown to be effective in reducing the risk of relapse in breast cancer (Mansour et al., 1998). According to current treatment guidelines, most node-positive patients receive adjuvant chemotherapy both in the US and Europe, because the risk of relapse is considerable. Nevertheless, not all patients do relapse, and there is a proportion of patients who would never have relapsed even without chemotherapy, but who nevertheless receive chemotherapy due to the currently used criteria. In hormone receptor positive patients, chemotherapy is usually given before endocrine treatment, whereas hormone receptor negative patients receive only chemotherapy.

The situation for node-negative patients is particularly complex. In the US, cytotoxic chemotherapy is recommended for node-negative patients, if the tumor is larger than 1 cm. In Europe, chemotherapy is considered for the node-negative cases if one or more risk factors is present, such as: tumor size larger than 2 cm; negative hormone receptor status; tumor grading of three; or age <35. Generally, there is a tendency to select premenopausal women for additional chemotherapy whereas for postmenopausal women, chemotherapy is often omitted. Compared to endocrine treatment, in particular that with Tamoxifen or aromatase inhibitors, chemotherapy is highly toxic, with short-term side effects such as nausea, vomiting, bone marrow depression, as well as long-term effect, such as cardiotoxicity and an increased risk for secondary cancers.

LONGFELT NEED IN THE ART. It is currently not clear which breast cancer patients should be selected for more aggressive therapy and which would do well without additional aggressive treatment, and thus clinicians agree that there is a substantial and unmet need for proper patient selection methods. The difficulty of selecting the right patients for adjuvant treatment, and of selecting the right adjuvant treatment, and the lack of suitable criteria is also reflected by a recent study and data, which showed that chemotherapy is used much less frequently than recommended (New Mexico Tumor registry; Du et al., 2003). This study provided substantial evidence that there is a need for better selection of patients for chemotherapy or other, more aggressive forms of breast cancer therapy.

THE PITX2 GENE. PITX2 (a.k.a. PTX2, RS, RGS, ARPI, Brx1, IDG2, IGDS, IHG2, RIEG, IGDS2, IRID2, Otlx2, RIEG1, MGC20144) is known to belong to the PTX subfamily of PTX1, PTX2, and PTX3 genes which define a novel family of transcription factors, within the paired-like class of homeodomain factors. The gene PITX2 (accession number NM_{—}153426) encodes the paired-like homeodomain transcription factor 2, which is known to be expressed during development of anterior structures such as the eye, teeth, and anterior pituitary.

Toyota et al., (Blood 97:2823-9, 2001) found hypermethylation of the PITX2 gene in a large proportion of acute myeloid leukemia. Furthermore, in this study hypermethylation of PITX2 is positively correlated to methylation of the ER gene and to a reduced expression level. Means to analyze the methylation pattern of the PITX2 gene have been described in a number of patent applications: WO 02/077272 relates to the use of methylation markers to differentiate between AML and ALL; WO 01/19845 relates to several differentially methylated sequences useful for diagnosis of several cell proliferative disorders; WO 02/00927 and WO 01/092565 relate to the use of methylation markers to diagnose diseases associated with development genes or associated with DNA transcription, respectively.

Loss of heterozygosity (hereinafter also referred to as “LOH”) of chromosome 4 is a known characteristic of many tumor types, and Shivapurkar et al. (Cancer Research 59, 3576-3580, 1999) have observed loss of heterozygosity at multiple regions of chromosome 4 in breast cancer samples and cell lines. Deletions at 4q25-26 were present in 67% of analyzed samples. However, the analyzed region (between markers D4S1586 and D4S175) does not map to the PITX2 gene, and no inference concerning PITX2 expression was made. Furthermore, the investigation as carried out does not indicate the suitability of any genes or loci of the region for a prognostic use.

Although the methylation of PITX2 has been associated with development, transcription and disease such as cancer, it has not heretofore been recognized as, or suggested as having a role in the outcome prediction of breast cancer patients, or for predicting responsiveness to endocrine treatment.

PRIOR ART EXPRESSSION ANALYSIS. The expression of a gene, or rather the protein encoded by the gene, can be studied on four different levels: firstly, protein expression levels can be determined directly; secondly, mRNA transcription levels can be determined; thirdly, epigenetic modifications, such as gene's DNA methylation profile or the gene's histone profile; can be analyzed, as methylation is often correlated with inhibited protein expression; and fourth, the gene itself may be analyzed for genetic modifications such as mutations, deletions, polymorphisms etc., which influence the expression of the gene product.

The levels of observation that have been studied by the methodological developments of recent years in molecular biology, are the genes themselves, the transcription of these genes into RNA, and the translation into the resulting proteins. However, how the activation and inhibition of specific genes, in specific cells and tissues, at specific time points in the course of development of an individual are controlled, is correlatable to the degree and character of the methylation of the genes or respectively the genome. In this respect, pathogenic conditions may manifest themselves in a changed methylation pattern of individual genes or of the genome.

Four terms generally apply to the fields of overall genome-wide analysis of all these biological processes: namely, Proteomics, Transcriptomics, Epigenomics (or Methylomics) and Genomics. Methods and techniques that can be used for studying expression or studying the modifications responsible for expression on all of these levels are well described in the literature and therefore known to a person skilled in the art. They are described in text books of molecular biology and in a large number of scientific journals.

Methods for analysis of protein expression of a single gene are known; typically requiring an antibody specific for the gene product of interest. Appropriate technologies are, for example, ELISA or Immunohistochemistry.

The analysis of mRNA levels has also has been adequately described, with the present ‘gold-standard’ being the use of reverse transcriptase PCR.

A more detailed description of the prior art relating to existing and well known technologies is given within the description of the present invention.

U.S. patent application 2003/0198970 by Gareth Roberts lists some of the technologies and methods relating to determine a person's “genetic make up” (i.e., the genetic modifications, such as deletions, polymorphisms, mutations etc., that may vary between and among individuals), and describes the potential role of this genetic sequence information in the individual's variability in disease, response to therapy and prognosis. Epigenetic differences however, are not mentioned. The gene PITX2 is listed within this application as one, out of a long list of about 2,500 other gene names, suggesting its expression could play a role in some kind of treatment response. However, this is merely an assumption, based on sheer speculation, becauses no experiments are disclosed, which demonstrate any kind of relation between genetic modifications of PITX2 and an individual's variation in treatment response.

PRIOR ART IN METHYLATION ANALYSIS. A less established area in this context is the field of epigenomics or epigenetics (i.e., the field concerned with analysis of DNA methylation patterns. 5-methylcytosine is the most frequent covalent base modification in the DNA of eukaryotic cells. Methylation of DNA can play an important role in the control of gene expression in mammalian cells. It plays a role, for example, in the regulation of the transcription, in genetic imprinting, and in tumorigenesis. DNA methyltransferases are involved in DNA methylation and catalyze the transfer of a methyl group from S-adenosylmethionine to cytosine residues to form 5-methylcytosine, a modified base that is found mostly at CpG sites in the genome. The presence of methylated CpG islands in the promoter region of genes can suppress their expression. This process may be due to the presence of 5-methylcytosine, which apparently interferes with the binding of transcription factors or other DNA-binding proteins to block transcription. In different types of tumors, aberrant or accidental methylation of CpG islands in the promoter region has been observed for many cancer-related genes, resulting in the silencing of their expression. Such genes include tumor suppressor genes, genes that suppress metastasis and angiogenesis, and genes that repair DNA (Momparler and Bovenzi, J. Cell Physiol. 183:145-54, 2000). Therefore, the identification of 5-methylcytosine as a component of genetic information is of considerable interest. However, 5-methylcytosine positions cannot be identified by sequencing, because 5-methylcytosine has the same base pairing behaviour as cytosine. Moreover, the epigenetic information carried by 5-methylcytosine is completely lost during PCR amplification.

METHYLATION ANALYSIS TECHNIQUES. Additionally, it has been described that DNA methylation may also play a role in the field of pharmacogenetics. An approach concerning the application of information concerning genetic modifications of the genome to the analysis of individual responses to treatment (e.g., similar to that described by Gareth Roberts in U.S. application 2003/0198970) is the subject of the application WO 02/037398, tailored to the application of information about epigenetic modifications of the genome, and based on DNA methylation analysis, to guide treatment selection and to study an individual's treatment responses.

Demonstration of the applicability of this idea was given, for example, by Esteller et al. (N Engl J. Med. 343:1350-4, 2000), who demonstrated that methylation of the MGMT promoter in gliomas is a useful predictor of the responsiveness of the tumors to alkylating agents. More recently, Frühwald has summarized a series of studies demonstrating that DNA methylation is associated with the aggressiveness of different cancers (Fruhwald M C. DNA methylation patterns in cancer: novel prognostic indicators? Am J Pharmacogenomics: 245-60, 2003).

An example of the potential of analysis of epigenetic modifications, such as DNA methylation analysis, to the prediction of treatment response related to breast cancer was presented by Martens et al. at the San Antonio Breast Cancer Symposium, San Antonio, Tex., Dec. 3-6, 2003. Breast cancer patients who were initially treated by surgical removal of tumors were treated for metastases using Tamoxifen. The primary tumor samples were analyzed for aberrant methylation patterns. The patients were then divided into two sub-classes according to their objective tumor response: patients with progressive disease (increasing metastasis size); and patients with complete or partial remission of the relapsed tumor (decreasing metastasis size). The two sub-classes could be distinguished on the basis of their methylation patterns. This indicates that the methylation pattern described in that study can serve as a predictive treatment response tool for an endocrine treatment (e.g., Tamoxifen). The results of this study, are the subject of patent application WO 04/035803, published on Apr. 29, 2004. and entitled “Method and nucleic acid for the improved treatment of breast cell proliferative disorders.” PITX2 is also listed as a predictive marker in said application, however the use of said marker is only described as a treatment response marker and not as a prognostic marker.

Currently several predictive markers are under evaluation. The only commonly used treatment targeting the endocrine pathways is Tamoxifen, however it is anticipated that the majority of biomarkers associated with Tamoxifen response are relevant to all drugs with the same mechanism of action, or that target the same pathway. For example, Estrogen receptor (hereinafter also referred to as ‘ER’) and Progesterone receptor (hereinafter also referred to as ‘PR’) expression are used to select patients for any treatment targeting the endocrine pathways. Among the markers which have been associated with Tamoxifen response is bcl-2. High bcl-2 expression levels showed promising correlation to Tamoxifen therapy response in patients with metastatic disease and prolonged survival and added valuable information to an ER negative patient subgroup (J Clin Oncology, 15 5:1916-1922, 1997; Endocrine, 13:1-10, 2000). There is conflicting evidence regarding the independent predictive value of c-erbB2 (Her2/neu) overexpression in patients with advanced breast cancer that require further evaluation and verification (British J of Cancer, 79:1220-1226, 1999; J Natl Cancer Inst, 90:1601-1608, 1998).

Other predictive markers include SRC-1 (steroid receptor coactivator-1), CGA mRNA over expression, cell kinetics and S phase fraction assays (Breast Cancer Res and Treat, 48:87-92, 1998; Oncogene, 20:6955-6959, 2001). Recently, uPA (Urokinase-type plasminogen activator) and PAI-1 (Plasminogen activator inhibitor type 1) together showed to be useful to define a subgroup of patients who have worse prognosis and who would benefit from adjuvant systemic therapy (J Clinical Oncology, 20 No. 4, 2002). However, all of these markers need further evaluations in prospective trials as none of them is yet a validated marker of response.

Additionally, study results presented by Paik et al. at the San Antonio Breast Cancer Symposium, San Antonio, Tex., Dec. 3-6, 2003, address this question, by analyzing the mRNA expression pattern of 16 genes plus 5 controls with RT-PCR. However it is unlikely that said markers will be suitable for use in a commercial test, due to the high number of genes. It is particularly preferred that for a commercially available test a more limited number of genes are analyzed.

A recent study relates to the prognostic power of methylation analysis in breast cancer patients (Müller et al. Cancer Res. 63:7641-5, 2003). Müller et al describe a set of genes that can be used as prognostic biomarkers in breast cancer patients by analysis of pre-therapeutic sera. Specific aberrant methylation patterns of two genes found in DNA from pre-treatment serum of cancer patients indicated whether their prognosis was good or bad. The DNA analyzed was not tissue derived DNA but serum DNA. Most likely, the presence of a tumor-specific pattern indicates that tumor derived DNA is present, however, the absence of a specific methylation pattern may be due to a tumor which does not show this methylation pattern, or a tumor which does not shed sufficient DNA into the blood stream. Good or bad prognosis was defined as long or short “overall survival” after surgery without adjuvant treatment. This result therefore relates to patients who do not receive a post-surgical treatment. The markers are therefore (unless proven otherwise) considered to be purely prognostic. The markers provide no information concerning treatment response and can provide only a very basic guide as to the aggressiveness of the tumor. On this basis clinicians can only speculate on the suitability of treatment options. As it is however standard to provide Tamoxifen (or other endocrine therapies) as an adjuvant treatment to the majority of patients irrespectively of the aggressiveness of the tumor, these markers are not applicable to most patients.

Therefore there is still a substantial and long-felt need in the art for the improved treatment of breast cancer patients that is not present in the current art. Specifically, none of the prior art markers is able to answer/address the specific problem as outlined above; namely, whether a patient treated by means of a primary treatment (e.g., surgery) is a suitable candidate for treatment using only an endocrine treatment (e.g., but not limited to Tamoxifen, or aromatase inhibitors) or if said patient would have a better prognosis if treated with a further adjuvant treatment (e.g., chemotherapy) instead of, or in addition to said endocrine treatment.

A purely prognostic marker for cancer patients which is irrespective of treatment, is not the preferred solution for the need in the art as described above. Although said markers provide some indication of the aggressiveness of the tumor and therefore may guide the selection of treatment that may be required they do not take into account the heterogeneity of cancers with respect to treatment response. Therefore, a patient with poor prognosis (determined using a purely prognostic markers) may respond well to adjuvant treatment with endocrine treatment, irrespective of the aggressiveness of the disease, however if a patient is a poor responder to said treatment, an alternative and/or additional treatment will be suitable for treatment even if said patient has a good prognosis.

### BRIEF DESCRIPTION OF THE DRAWINGS

FIG. 1 illustrates a simplified model of a Stage 1-3 breast tumor wherein primary treatment was surgery (at point 1), followed by adjuvant therapy with Tamoxifen, as an example for an endocrine treatment. The Y axis represents tumor(s) mass (or size), wherein the line ‘3’ indicates the limit of detectability of said tumor mass. The X axis represents time. In a first scenario a patient without relapse during endocrine treatment (4) is shown as remaining below the limit of detectability for the duration of the observation. A patient with relapse of the cancer (5) has a period of disease free survival (2) followed by relapse when the carcinoma mass reaches the level of detectability.

FIG. 2 shows the result of the assay (QM assay) as described in Example 4: A Kaplan-Meier estimated metastasis-free survival curve for three CpG sites of the PITX2 gene by means of Real-Time methylation specific probe analysis (QM assay). The lower curve shows the proportion of metastasis free patients in the population with above median methylation levels, the upper curve shows the proportion of metastasis free patients in the population with below median methylation levels. The X axis shows the metastasis free survival times of the patients in months, and the Y axis shows the proportion of metastasis free survival patients.

FIG. 3 shows the result of the chip hybridization experiment as described in Example 2. A Kaplan-Meier estimated metastasis-free survival curves for two CpG positions of the PITX2 gene by means of methylation specific detection oligonucleotide hybridization analysis. The lower curve shows the proportion of metastasis free patients in the population with above median methylation levels, the upper curve shows the proportion of metastasis free patients in the population with below median methylation levels. The X axis shows the metastasis free survival times of the patients in months, and the Y axis shows the proportion of metastasis free survival patients.

FIG. 4 shows the Kaplan-Meier estimated metastasis-free survival curves for two CpG positions of the PITX2 gene by means of methylation specific detection oligonucleotide hybridization analysis. The lower line shows the proportion of metastasis free patients in the population of 55 patients with above median methylation levels, the upper curve shows the proportion of metastasis free patients in the population of 54 patients with below median methylation levels. The X axis shows the metastasis free survival times of the patients in years, and the Y axis shows the proportion of metastasis free survival patients in %. This resulted from a first data set that was achieved in a first study.

FIG. 5 shows the Kaplan-Meier estimated metastasis-free survival curves for six different CpG positions located within the preferred region of the PITX2 gene (SEQ ID NO:13) by means of methylation specific detection oligonucleotide hybridization analysis. The lower line shows the proportion of metastasis free patients in the population of 118 patients with above median methylation levels, the upper curve shows the proportion of metastasis free patients in the population of 118 patients with below median methylation levels. The X axis shows the metastasis free survival times of the patients in years, and the Y axis shows the proportion of metastasis free survival patients in %. This resulted from a second data set that was achieved in a second study.

FIG. 6 shows the Kaplan-Meier estimated metastasis-free survival curves for 6 different CpG positions located within the preferred region of the PITX2 gene (SEQ ID NO:13) by means of methylation specific detection oligonucleotide hybridization analysis. This time only a sub-population of 148 patients, characterized by a tumor at grade G1 or G2, was analyzed: The lower curve shows the proportion of metastasis free patients in the population of 74 patients with above median methylation levels, the upper curve shows the proportion of metastasis free patients in the population of 74 patients with below median methylation levels. The X axis shows the metastasis free survival times of the patients in years, and the Y axis shows the proportion of metastasis free survival patients in %. This resulted from a second data set as shown in the Example 2.

FIG. 7 shows the Kaplan-Meier estimated metastasis-free survival curves for 4 different CpG positions located within the preferred region of the PITX2 gene (SEQ ID NO: 13) by means of methylation specific detection oligonucleotide hybridization analysis. This time a sub-population of 224 patients, characterized by a tumor of stage 1 or 2 (T1 or T2), was analyzed: The lower curve shows the proportion of metastasis free patients in the population of 112 patients with above median methylation levels, the upper curve shows the proportion of metastasis free patients in the population of 112 patients with below median methylation levels. The X axis shows the metastasis free survival times of the patients in years, and the Y axis shows the proportion of metastasis free survival patients in %. This resulted from the second data set that was achieved in the second example.

FIG. 8 shows the disease-free survival curves of a combination of two oligonucleotides each from the genes TBC1D3 and CDK6, and one oligonucleotide from the gene PITX2 covering two CpG sites. The black curve shows the proportion of disease free patients in the population with above median methylation scores, the gray curve shows the proportion of disease free patients in the population with below median methylation scores.

FIG. 9 shows the plot according to FIG. 8 and the classification of the sample set by means of the St. Gallen method. The unbroken lines represent the methylation analysis wherein the black curve shows the proportion of disease free patients in the population with above median methylation scores, the gray curve shows the proportion of disease free patients in the population with below median methylation scores. The broken lines represent the St. Gallen classification of the sample set wherein the black curve shows the disease free survival time of the high risk group and the gray curve shows the disease free survival of the low risk group.

FIG. 10 illustrates the amino acid sequence of the polypeptide encoded by the gene PITX2.

FIG. 11 illustrates the positions of the amplificates sequenced in Example 7. ‘A’ shows an illustration of the gene with the major exons annotated, ‘B’ shows annotated mRNA transcript variants and ‘C’ shows CpG rich regions of the gene. The positions of Amplificates 1 to 11 are shown to the right of the illustrations.

FIG. 12 shows the sequencing data of 11 amplificates of the gene PITX2 according to Example 7. Each column of the matrices of columns ‘A’ and ‘B’ represent the sequencing data for one amplificate. The amplificate number is shown to the left of the matrices. Each row of a matrix represents a single CpG site within the fragment and each column represents an individual DNA sample. The matrices in the column marked ‘A’ showed below median methylation as measured by QM assays, the matrices in the column marked ‘B’ showed below median methylation as measured by QM assays.

The bar on the left represents a scale of the percent methylation, with the degree of methylation represented by the shade of each position within the column from black representing 100% methylation to light gray representing 0% methylation. White positions represented a measurement for which no data was available.

FIG. 13 shows a schematic view of mRNA transcript variants of PITX2, as annotated in the on-line Ensembl database.

FIG. 14 shows the Kaplan-Meier estimated disease-free survival curves for a CpG position of the ERBB2 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 15 shows the Kaplan-Meier estimated metastasis-free survival curves for a CpG position of the ERBB2 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 16 shows the Kaplan-Meier estimated metastasis-free survival curves for a CpG position of the TFF1 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 17 shows the Kaplan-Meier estimated metastasis-free survival curves for a CpG position of the TFF1 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 18 shows the Kaplan-Meier estimated disease-free survival curves for a CpG position of the PLAU gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 19 shows the Kaplan-Meier estimated metastasis-free survival curves for a CpG position of the PLAU gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 20 shows the Kaplan-Meier estimated disease-free survival curves for a CpG position of the PITX2 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 21 shows the Kaplan-Meier estimated metastasis-free survival curves for a CpG position of the PITX2 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 22 shows the Kaplan-Meier estimated disease-free survival curves for a CpG position of the TBC1D3 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 23 shows the Kaplan-Meier estimated metastasis-free survival curves for a CpG position of the TBC1D3 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 24 shows the Kaplan-Meier estimated disease-free survival curves for a CpG position of the ERBB2 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 25 shows the Kaplan-Meier estimated metastasis-free survival curves for a CpG position of the ERBB2 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 26 shows the Kaplan-Meier estimated disease-free survival curves for a CpG position of the TFF1 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 27 shows the Kaplan-Meier estimated metastasis-free survival curves for a CpG position of the TFF1 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 28 shows the Kaplan-Meier estimated disease-free survival curves for a CpG position of the PLAU gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 29 shows the Kaplan-Meier estimated metastasis-free survival curves for a CpG position of the PLAU gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 30 shows the Kaplan-Meier estimated disease-free survival curves for a CpG position of the PITX gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 31 shows the Kaplan-Meier estimated metastasis-free survival curves for a CpG position of the PITX gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 32 shows the Kaplan-Meier estimated disease-free survival curves for a CpG position of the PITX gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 33 shows the Kaplan-Meier estimated metastasis-free survival curves for a CpG position of the PITX gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 34 shows the Kaplan-Meier estimated disease-free survival curves for a CpG position of the ONECUT gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 35 shows the Kaplan-Meier estimated metastasis-free survival curves for a CpG position of the ONECUT gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 36 shows the Kaplan-Meier estimated disease-free survival curves for a CpG position of the TBC1D3 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 37 shows the Kaplan-Meier estimated metastasis-free survival curves for a CpG position of the TBC1D3 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 38 shows the Kaplan-Meier estimated disease-free survival curves for a CpG position of the ABCA8 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 39 shows the Kaplan-Meier estimated metastasis-free survival curves for a CpG position of the ABCA8 gene by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 40 shows the Kaplan-Meier estimated disease-free survival curves for a CpG position of a combination of the TFF1 & PLAU genes by means of Real-Time methylation specific probe analysis according to Example 8. he X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 41 shows the Kaplan-Meier estimated metastasis-free survival curves for a CpG position of a combination of the TFF1 & PLAU genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 42 shows the Kaplan-Meier estimated disease-free survival curves for a CpG position of a combination of the TFF1 & PLAU & PITX genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 43 shows the Kaplan-Meier estimated metastasis-free survival curves for a CpG position of a combination of the TFF1 & PLAU & PITX genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 44 shows the Kaplan-Meier estimated disease-free survival curves for a CpG position of a combination of the PITX & TFF1 genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 45 shows the Kaplan-Meier estimated metastasis-free survival curves for a CpG position a combination of the PITX & TFF1 genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 46 shows the Kaplan-Meier estimated disease-free survival curves for a CpG position of a combination of the PITX & PLAU genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 47 shows the Kaplan-Meier estimated metastasis-free survival curves for a CpG position of a combination of the PITX & PLAU genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 48 shows the Kaplan-Meier estimated disease-free survival curves for a CpG position of a combination of the TFF1 & PLAU genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 49 shows the Kaplan-Meier estimated metastasis-free survival curves for a CpG position of a combination of the TFF1 & PLAU genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 50 shows the Kaplan-Meier estimated disease-free survival curves for a CpG position of a combination of the TFF1 & PLAU & PITX genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 51 shows the Kaplan-Meier estimated metastasis-free survival curves for a CpG position of a combination of the TFF1 & PLAU & PITX genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 52 shows the Kaplan-Meier estimated disease-free survival curves for a CpG position of a combination of the PITX & TFF1 genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 53 shows the Kaplan-Meier estimated metastasis-free survival curves for a CpG position of a combination of the PITX & TFF1 genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels.

FIG. 54 shows the Kaplan-Meier estimated disease-free survival curves for a CpG position of a combination of the PITX & PLAU genes by means of Real-Time methylation specific probe analysis according to Example 8. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

FIG. 55 shows the Kaplan-Meier estimated metastasis-free survival curves for a CpG position of a combination of the PITX & PLAU genes by means of Real-Time methylation specific probe analysis according to Example 8. The X axis shows the disease free survival times of the patients in years, and the Y-axis shows the proportion of patients with metastasis free survival. The black plot shows the proportion of metastasis free patients in the population with above an optimized cut off point's methylation levels, the gray plot shows the proportion of disease free patients in the population with below an optimized cut off point's methylation levels.

FIG. 56 shows a scatter plot of matched pair PET and fresh frozen tissues analyzed using PITX2 gene assay 1 according to Example 8. Quantitative methylation CT scores of PET samples are shown on the Y-axis, and quantitative methylation CT scores of fresh frozen samples are shown on the X-axis. The association between the paired samples is 0.81 (Spearman's rho). This analysis is based on n=89 samples.

FIG. 57 shows the Disease free survival (DFS) of randomly selected ER+, N0, untreated patient population in Kaplan-Meier survival plot according to Example 8. Proportion of disease free patients is shown on the Y-axis and time in years is shown on the X-axis. 139 events were observed (observed event rate=33%). Disease free survival after 5 years: 74.5% [70.3%, 78.9%], after 10 years 59.8% [54.2%, 66%]. 95% confidence intervals are plotted.

FIG. 58 shows the distribution of follow-up times in ER+, N0, untreated population according to Example 8. Frequency is shown on the Y-axis and time in months is shown on the X-axis. The figure on the left shows patients with event (all kinds of relapses). Mean follow-up time 45.8 months (standard deviation=31), median=38 (range=[2, 123]). The figure on the right shows censored patients. Mean follow up time 93 months (standard deviation=35.6), median=94 (range=[1, 190]).

FIG. 59 shows the Disease free survival (DFS) of ER+, N0, TAM treated population in Kaplan-Meier plot according to Example 8. Proportion of disease free patients is shown on the Y-axis and time in years is shown on the X-axis. 56 events were observed (observed event rate=10%). DFS after 5 years: 92.4% [90%, 94.9%], after 10 years: 82.1% [77.3%, 87.2%]. 95% confidence intervals are plotted.

FIG. 60 shows the distribution of follow-up times in ER+, N0, untreated population according to Example 8. Frequency is shown on the Y-axis and time in months is shown on the X-axis. The figure on the left shows patients with all events (all kinds of relapses). Mean follow-up time 47.9 months (standard deviation=24.4), median=45 (range=[2, 98]). The figure on the right shows censored patients. Mean follow up time 65.3 months (standard deviation=31.6), median=64 (range=[0, 158]).

FIG. 61 shows the ROC plot at different times for marker model 3522 (Assay 1) and 2265 on ER+N0 TAM treated population according to Example 8. FIG. A shows the plot at 60 months, FIG. B shows the plot at 72 months, FIG. C shows the plot at 84 months and FIG. D shows the plot at 96 months. Only distant metastasis are defined as events. Sensitivity (proportion of all relapsed patients in poor prognostic group) shown on the X-axis and specificity (proportion of all relapse free patients in good prognostic group) shown on the Y-axis are calculated from KM estimates, and the estimated area under the curve (AUC) is calculated. Values for median cut off (triangle) and best cut off (diamond, 0.32 quantile) are plotted.

FIG. 62 shows the ROC plot at different times for marker model 3522 (Assay 1) alone on ER+N0 TAM treated population according to Example 8. FIG. A shows the plot at 60 months, FIG. B shows the plot at 72 months, FIG. C shows the plot at 84 months and FIG. D shows the plot at 96 months. Only distant metastasis are defined as events. Sensitivity (proportion of all relapsed patients in poor prognostic group) shown on the X-axis and specificity (proportion of all relapse free patients in good prognostic group) shown on the Y-axis are calculated from KM estimates, and the estimated area under the curve (AUC) is calculated. Values for median cut off (triangle) and best cut off (diamond, 0.42 quantile) are plotted.

FIG. 63 shows the ROC plot at different times for marker model 2265 on ER+N0 TAM treated population according to Example 8. FIG. A shows the plot at 60 months, FIG. B shows the plot at 72 months, FIG. C shows the plot at 84 months and FIG. D shows the plot at 96 months. Only distant metastasis are defined as events. Sensitivity (proportion of all relapsed patients in poor prognostic group) shown on the X-axis and specificity (proportion of all relapse free patients in good prognostic group) shown on the Y-axis are calculated from KM estimates for different thresholds (=5, 6, 7, 8 years) and the estimated area under the curve (AUC) is calculated. Values for median cut off (triangle) and best cut off (diamond, 0.78 quantile) are plotted.

FIG. 64 shows the ROC plot at different times for marker model 2395 on ER+N0 TAM treated population according to Example 8. FIG. A shows the plot at 60 months, FIG. B shows the plot at 72 months, FIG. C shows the plot at 84 months and FIG. D shows the plot at 96 months. Only distant metastasis are defined as events. Sensitivity (proportion of all relapsed patients in poor prognostic group) shown on the X-axis and specificity (proportion of all relapse free patients in good prognostic group) shown on the Y-axis are calculated from KM estimates for different thresholds (=5, 6, 7, 8 years), and the estimated area under the curve (AUC) is calculated. Values for median cut off (triangle) and best cut off (diamond, 0.77 quantile) are plotted.

### SUMMARY OF THE INVENTION

In a preferred aspect, the present invention provides a prognostic marker, PITX2 (which shall be recognized as the gene encoding for the protein PITX2; and the mRNA transcript thereof being described in accession number NM_{—}153426), which, however, is not ‘purely prognostic’. This marker provides a solution to the need in the art as outlined above, by providing guiding information on the question of whether or not an adjuvant chemotoxic therapy shall be subscribed in addition to treatment with endocrines, like tamoxifen, or whether this is an unnecessary burden to the patient.

It is herein disclosed that aberrant expression of the gene PITX2 is correlated to prognosis and/or predicted outcome of endocrine (e.g., estrogen) treatment of breast cell proliferative disorder patients, in particular breast carcinoma.

This marker thereby provides a novel and highly beneficial means for the characterization of breast carcinomas. Aberrant expression of the gene PITX2 is indicative of the relapse and/or survival of a breast carcinoma patient. The herein described invention is thereby particularly useful for making improved treatment decisions.

The marker is also indicative of the relapse and/or survival of said patients when treated with one or more treatments which target the estrogen receptor, synthesis or conversion pathways or are otherwise involved in estrogen metabolism, production or secretion.

The herein described invention is thereby particularly useful for the differentiation of individuals who may be appropriately treated with one or more treatments which target the estrogen receptor pathway or are involved in estrogen metabolism, production or secretion from those individuals who would be optimally treated with other treatments in addition to or instead of said treatment. Preferred ‘other treatments’ include but are not limited to chemotherapies or radiotherapies

Accordingly it is particularly preferred that said marker be used in the treatment of breast cancer patients by enabling the classification of patients according to their likely treatment outcome wherein said patients are treated with an adjuvant therapy targeting the endocrine pathways. It is further preferred that patients with a poor treatment outcome are provided with a further adjuvant treatment instead of or in addition to said endocrine therapy, in particular but not limited to chemotherapy. A marker suitable for said purpose shall hereinafter also be referred to as an ‘adjuvant marker.’

This invention also relates to the use of PITX2, as an ‘adjuvant marker’, which also serves as a ‘prognostic marker’, especially in hormone receptor negative women, which would not get any endocrine treatment at all.

### DETAILED DESCRIPTION OF THE INVENTION

Characterization of a breast cancer in terms of prognosis and/or treatment outcome enables the physician to make an informed decision as to a therapeutic regimen with appropriate risk and benefit trade off's to the patient.

In the context of the present invention the terms “estrogen receptor positive” and/or “progesterone receptor positive” when used to describe a breast cell proliferative disorder are taken to mean that the proliferating cells express said hormone receptor.

In the context of the present invention the term ‘aggressiveness’ is taken to mean one or more of: high likelihood of relapse post surgery; below average or below median patient survival; below average or below median disease free survival; below average or below median relapse-free survival; above average tumor-related complications; fast progression of tumor or metastases. According to the aggressiveness of the disease an appropriate treatment or treatments may be selected from the group consisting of chemotherapy, radiotherapy, surgery, biological therapy, immunotherapy, antibody treatments, treatments involving molecularly targeted drugs, estrogen receptor modulator treatments, estrogen receptor down-regulator treatments, aromatase inhibitors treatments, ovarian ablation, treatments providing LHRH analogues or other centrally acting drugs influencing estrogen production. Wherein a cancer is characterized as ‘aggressive’ it is particularly preferred that a treatment such as, but not limited to, chemotherapy is provided in addition to or instead of an endocrine targeting therapy.

Indicators of tumor aggressiveness standard in the art include but are not limited to, tumor stage, tumor grade, nodal status and survival.

Unless stated otherwise as used herein the term “survival” shall be taken to include all of the following: survival until mortality, also known as overall survival (wherein said mortality may be either irrespective of cause or breast tumor related); “recurrence-free survival” (wherein the term recurrence shall include both localized and distant recurrence); metastasis free survival; disease free survival (wherein the term disease shall include breast cancer and diseases associated therewith). The length of said survival may be calculated by reference to a defined start point (e.g., time of diagnosis or start of treatment) and end point (e.g., death, recurrence or metastasis).

As used herein the term “prognostic marker” shall be taken to mean an indicator of the likelihood of progression of the disease, in particular aggressiveness and metastatic potential of a breast tumor.

As used herein the term ‘predictive marker’ shall be taken to mean an indicator of response to therapy, said response is preferably defined according to patient survival. It is preferably used to define patients with high, low and intermediate length of survival or recurrence after treatment, that is the result of the inherent heterogeneity of the disease process.

As defined herein the term ‘predictive marker’ may in some situations fall within the remit of a herein described ‘prognostic marker’, for example, wherein a prognostic marker differentiates between patients with different survival outcomes pursuant to a treatment, said marker is also a predictive marker for said treatment. Therefore, unless otherwise stated the two terms shall not be taken to be mutually exclusive.

As used herein the term ‘expression’ shall be taken to mean the transcription and translation of a gene, as well as the genetic or the epigenetic modifications of the genomic DNA associated with the marker gene and/or regulatory or promoter regions thereof. Genetic modifications include SNPs, point mutations, deletions, insertions, repeat length, rearrangements and other polymorphisms. The analysis of either the expression levels of protein, or mRNA or the analysis of the patient's individual genetic or epigenetic modification of the marker gene are herein summarized as the analysis of ‘expression of the gene.

The level of expression of a gene may be determined by the analysis of any factors associated with or indicative of the level of transcription and translation of a gene including but not limited to methylation analysis, loss of heterozygosity (hereinafter also referred to as LOH), RNA expression levels and protein expression levels.

Furthermore the activity of the transcribed gene may be affected by genetic variations such as but not limited to genetic modifications (including but not limited to SNPs, point mutations, deletions, insertions, repeat length, rearrangements and other polymorphisms).

The terms “endocrine therapy” or “endocrine treatment” are meant to comprise any therapy, treatment or treatments targeting the estrogen receptor pathway or estrogen synthesis pathway or estrogen conversion pathway, which is involved in estrogen metabolism, production or secretion. Said treatments include, but are not limited to estrogen receptor modulators, estrogen receptor down-regulators, aromatase inhibitors, ovarian ablation, LHRH analogues and other centrally acting drugs influencing estrogen production.

The term “monotherapy” shall be taken to mean the use of a single drug or other therapy.

In the context of the present invention the term “chemotherapy” is taken to mean the use of pharmaceutical or chemical substances to treat cancer. This definition excludes radiation therapy (treatment with high energy rays or particles), hormone therapy (treatment with hormones or hormone analogues) and surgical treatment.

In the context of the present invention the term “adjuvant treatment” is taken to mean a therapy of a cancer patient immediately following an initial non-chemotherapeutical therapy, (e.g., surgery). In general, the purpose of an adjuvant therapy is to decrease the risk of recurrence.

In the context of the present invention the term “determining a suitable treatment regimen for the subject” is taken to mean the determination of a treatment regimen (i.e., a single therapy or a combination of different therapies that are used for the prevention and/or treatment of the cancer in the patient) for a patient that is started, modified and/or ended based or essentially based or at least partially based on the results of the analysis according to the present invention. One example is starting an adjuvant endocrine therapy after surgery, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

In the context of this invention the terms “obtaining a biological sample” or “obtaining a sample from a subject”, shall not be taken to include the active retrieval of a sample from an individual, (e.g., the performance of a biopsy). Said terms shall be taken to mean the obtainment of a sample previously isolated from an individual. Said samples may be isolated by any means standard in the art, including but not limited to biopsy, surgical removal, body fluids isolated by means of aspiration. Furthermore said samples may be provided by third parties including but not limited to clinicians, couriers, commercial sample providers and sample collections.

In the context of the present invention, the term “CpG island” refers to a contiguous region of genomic DNA that satisfies the criteria of (I) having a frequency of CpG dinucleotides corresponding to an “Observed/Expected Ratio”>0.6, and (2) having a “GC Content”>0.5. CpG islands are typically, but not always, between about 0.2 to about 1 kb in length.

In the context of the present invention the term “regulatory region” of a gene is taken to mean nucleotide sequences which affect the expression of a gene. Said regulatory regions may be located within, proximal or distal to said gene. Said regulatory regions include but are not limited to constitutive promoters, tissue-specific promoters, developmental-specific promoters, inducible promoters and the like. Promoter regulatory elements may also include certain enhancer sequence elements that control transcriptional or translational efficiency of the gene.

In the context of the present invention, the term “methylation” refers to the presence or absence of 5-methylcytosine (“5-mCyt”) at one or a plurality of CpG dinucleotides within a DNA sequence.

In the context of the present invention the term “methylation state” is taken to mean the degree of methylation present in a nucleic acid of interest, this may be expressed in absolute or relative terms (i.e., as a percentage or other numerical value) or by comparison to another tissue and therein described as hypermethylated, hypomethylated or as having significantly similar or identical methylation status.

In the context of the present invention, the term “hemi-methylation” or “hemimethylation” refers to the methylation state of a CpG methylation site, where only a single cytosine in one of the two CpG dinucleotide sequences of the double stranded CpG methylation site is methylated (e.g., 5′-NNC^{M}GNN-3′ (top strand): 3′-NNGCNN-5′ (bottom strand)).

In the context of the present invention, the term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

In the context of the present invention, the term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

In the context of the present invention, the term “microarray” refers broadly to both “DNA microarrays,” and ‘DNA chip(s),’ as recognized in the art, encompasses all art-recognized solid supports, and encompasses all methods for affixing nucleic acid molecules thereto or synthesis of nucleic acids thereon.

“Genetic parameters” are mutations and polymorphisms of genes and sequences further required for their regulation. To be designated as genetic modifications or mutations are, in particular, insertions, deletions, point mutations, inversions and polymorphisms and, particularly preferred, SNPs (single nucleotide polymorphisms).

“Epigenetic modifications” or “epigenetic parameters” are modifications of DNA bases of genomic DNA and sequences further required for their regulation, in particular, cytosine methylations thereof. Further epigenetic parameters include, for example, the acetylation of histones which, however, cannot be directly analyzed using the described method but which, in turn, correlate with the DNA methylation.

In the context of the present invention, the term “bisulfite reagent” refers to a reagent comprising bisulfite, disulfite, hydrogen sulfite or combinations thereof, useful as disclosed herein to distinguish between methylated and unmethylated CpG dinucleotide sequences.

In the context of the present invention, the term “Methylation assay” refers to any assay for determining the methylation state of one or more CpG dinucleotide sequences within a sequence of DNA.

In the context of the present invention, the term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

In the context of the present invention, the term “MethyLight™” refers to the art-recognized fluorescence-based real-time PCR technique described by Eads et al., Cancer Res. 59:2302-2306, 1999.

In the context of the present invention, the term “HeavyMethyl™” assay, in the embodiment thereof implemented herein, refers to a methylation assay comprising methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

In the context of the present invention the term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

In the context of the present invention the term “COBRA” (Combined Bisulfite Restriction Analysis) refers to the art-recognized methylation assay described by Xiong & Laird, Nucleic Acids Res. 25:2532-2534, 1997.

In the context of the present invention the term “hybridization” is to be understood as a bond of an oligonucleotide to a complementary sequence along the lines of the Watson-Crick base pairings in the sample DNA, forming a duplex structure.

“Stringent hybridization conditions,” as defined herein, involve hybridizing at 68° C. in 5×SSC/5× Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

“Background DNA” as used herein refers to any nucleic acids which originate from sources other than breast cells.

Using the methods and nucleic acids described herein, statistically significant models of patient relapse, disease free survival, metastasis free survival, overall survival and/or disease progression can be developed and utilized to assist patients and clinicians in determining suitable treatment options to be included in the therapeutic regimen.

In one aspect the method provides a prognostic marker for a cell proliferative disorder of the breast tissues. Preferably this prognosis is provided in terms of an outcome selected from the group consisting of likelihood of relapse; overall patient survival; metastasis free survival; disease free survival or disease progression.

In a further aspect of the invention said marker is used as a predictive marker of outcome of a treatment which targets the estrogen receptor pathway or is involved in estrogen metabolism, production or secretion as a therapy for patients suffering from a cell proliferative disorder of the breast tissues. This aspect of the method enables the physician to determine which treatments may be used in addition to or instead of said endocrine treatment. It is preferred that said additional treatment is a more aggressive therapy such as, but not limited to, chemotherapy. Thus, the present invention will be seen to reduce the problems associated with present breast cell proliferative disorder prognostic, predictive and treatment response prediction methods.

Using the methods and nucleic acids as described herein, patient survival can be evaluated before or during treatment for a cell proliferative disorder of the breast tissues, in order to provide critical information to the patient and clinician as to the likely progression of the disease. It will be appreciated, therefore, that the methods and nucleic acids exemplified herein can serve to improve a patient's quality of life and odds of treatment success by allowing both patient and clinician a more accurate assessment of the patient's treatment options.

The herein disclosed method may be used for the improved treatment of all breast cell proliferative disorder patients, both pre- and post-menopausal and independent of their node or estrogen receptor status. However, it is particularly preferred that said patients are node-negative and estrogen receptor positive.

The present invention makes available a method for the improved treatment of breast cell proliferative disorders, by enabling the improved prediction of a patient's survival, in particular by predicting the likelihood of relapse post-surgery, both with or without adjuvant endocrine treatment. Furthermore, the present invention provides a means for the improved prediction of treatment outcome with endocrine therapy, wherein said therapy comprises one or more treatments which target the estrogen receptor pathway or are involved in estrogen metabolism, production, or secretion.

The method according to the invention may be used for the analysis of a wide variety of cell proliferative disorders of the breast tissues including, but not limited to, ductal carcinoma in situ, invasive ductal carcinoma, invasive lobular carcinoma, lobular carcinoma in situ, comedocarcinoma, inflammatory carcinoma, mucinous carcinoma, scirrhous carcinoma, colloid carcinoma, tubular carcinoma, medullary carcinoma, metaplastic carcinoma, and papillary carcinoma and papillary carcinoma in situ, undifferentiated or anaplastic carcinoma and Paget's disease of the breast.

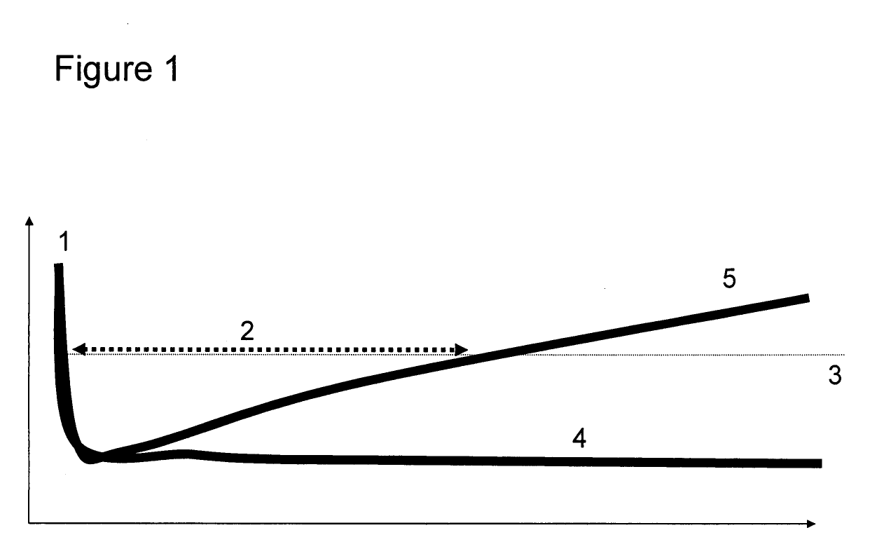

The method according to the invention may be used to provide a prognosis of breast cell proliferative disorder patients, furthermore said method may be used to provide a prediction of patient survival and/or relapse following treatment by endocrine therapy.

Wherein the herein disclosed markers, methods and nucleic acids are used as prognostic markers it is particularly preferred that said prognosis is defined in terms of patient survival and/or relapse. In this embodiment, patients survival times and/or relapse are predicted according to their gene expression or genetic or epigenetic modifications thereof. In this aspect of the invention it is particularly preferred that said patients are tested prior to receiving any adjuvant endocrine treatment.

Wherein the herein disclosed markers, methods and nucleic acids are used as predictive markers it is particularly preferred that the method is applied to predict the outcome of patients who receive endocrine treatment as secondary treatment to an initial non chemotherapeutical therapy, for example, surgery (hereinafter referred to as the ‘adjuvant setting’) as illustrated in FIG. 1. Such a treatment is often prescribed to patients suffering from Stage 1 to 3 breast carcinomas. It is also preferred that said ‘outcome’ is defined in terms of patients survival and/or relapse.

In this embodiment, patients survival times and/or relapse are predicted according to their gene expression or genetic or epigenetic modifications thereof. By detecting patients with below average or below median metastasis free survival or disease free survival times and/or high likelihood of relapse the physician may choose to recommend the patient for further treatment, instead of or in addition to the endocrine targeting therapy(s), in particular but not limited to, chemotherapy.

Aspects of the herein described invention provide, inter alia, a novel breast cell proliferative disorder prognostic and predictive biomarker.

It is herein described that aberrant expression of the gene PITX2 and/or regulatory or promoter regions thereof is correlated to prognosis and/or prediction of outcome of estrogen treatment of breast cell proliferative disorder patients, in particular breast carcinoma.

This marker thereby provides a novel means for the characterization of breast cell proliferative disorders. As described herein determination of the expression of the gene PITX2 and/or regulatory or promoter regions thereof enables the prediction of prognosis of a patient with a proliferative disorder of the breast tissues.

In an alternative embodiment, the expression of the gene PITX2 and/or regulatory or promoter regions thereof enables the prediction of treatment response of a patient treated with one or more treatments which target the estrogen receptor, synthesis or conversion pathways or are otherwise involved in estrogen metabolism, production or secretion.

The herein described invention is thereby useful for the differentiation of individuals who may be appropriately treated with one or more treatments which target the estrogen receptor pathway or are involved in estrogen metabolism, production or secretion from those individuals, who would be optimally treated with other treatments in addition to said treatment.

Preferred ‘other treatments’ include but are not limited to chemotherapy or radiotherapy. It is particularly preferred that said prognosis and/or treatment response is stated in terms of likelihood of relapse, survival or outcome.

In a further embodiment of the invention, the aberrant expression of a plurality of genes comprising the gene PITX2 and/or regulatory or promoter regions thereof is analyzed. Said plurality of genes is hereinafter also referred to as a ‘gene panel’. The analysis of multiple genes increases the accuracy of a provided prognosis and/or prediction of estrogen treatment outcome. It is preferred that the gene panel consists of up to seven genes and/or their promoter regions associated with prognosis and/or prediction of treatment response of breast carcinoma patients. It is further preferred that said panel consists of the gene PITX2 and one or more genes selected from the group consisting of ABCA8, CDK6, ERBB2, ONECUT2, PLAU, TBC1D3 and TFF1 and/or regulatory regions thereof.\It is particularly preferred that the gene panel is selected from the group of gene panels consisting of:
PITX2, PLAU and TFF1;
PITX2 and PLAU; and
PITX2 and TFF1.

It is particularly preferred that the gene panel consisting PITX2 and TFF1 is used to predict outcome of treatment of patients with an endocrine treatment. It is particularly preferred that the gene panel consisting PITX2 and PLAU is used to provide a prognosis of patients. It is preferred that said patients are analyzed prior to receiving any endocrine treatment.

In further embodiments this invention relates to new methods and sequences for the prognosis of patients diagnosed with breast cell proliferative disease.

In yet a further aspect, the invention relates to new methods and sequences, which may be used as tools for the selection of suitable treatments of patients diagnosed with breast cell proliferative disease based on a prediction of likelihood of relapse, survival or outcome.

More specifically, aspects of this invention provide new methods and sequences for patients diagnosed with breast cell proliferative disease, allowing the improved selection of suitable adjuvant therapy. Furthermore, it is preferred that patients with poor prognosis following endocrine monotherapy are provided with chemotherapy in addition to or instead of an endocrine therapy.

One aspect of the invention is the provision of methods for providing a prognosis and/or prediction of outcome of endocrine treatment of a patient with a cell proliferative disorder of the breast tissues. Preferably said prognosis and/or prediction is provided in terms of likelihood of relapse or the survival of said patient. It is further preferred that said survival is disease free survival or metastasis free survival. It is also preferred that said disease is breast cancer. These methods comprise the analysis of the expression levels of the gene PITX2 and/or regulatory regions thereof.

In further embodiments the method comprises analysis of the expression of a ‘gene panel’ comprising the gene PITX2 and one or more genes selected from the group consisting of ABCA8, CDK6, ERBB2, ONECUT2, PLAU, TBC1D3 and TFF1 and/or regulatory regions thereof. It is particularly preferred that said gene panels are selected from the group of gene panels consisting of:
PITX2, PLAU and TFF1
PITX2 and PLAU
PITX2 and TFF1

It is particularly preferred that the expression of the gene panel consisting PITX2 and TFF1 is determined in order to predict outcome of treatment of patients with an endocrine treatment. It is also particularly preferred that the expression of the gene panel consisting PITX2 and PLAU is determined in order to provide a prognosis of patients. It is preferred that said patients are analyzed prior to receiving any endocrine treatment.

Determination of expression may be achieved by any means standard in the art. However, it is most preferably achieved by analysis of LOH, methylation, protein expression, mRNA expression, genetic or other epigenetic modifications of the genomic sequences.

Especially preferred is the analysis of the DNA methylation profile of the genomic sequence of the gene PITX2 and/or regulatory or promoter regions thereof as given in SEQ ID NO:149. Further preferred is the analysis of the methylation status of CpG positions within the following sections of SEQ ID NO:149: nucleotide 2,700-nucleotide 3,000; nucleotide 3,900-nucleotide 4,200; nucleotide 5,500-nucleotide 8,000; nucleotide 13,500-nucleotide 14,500; nucleotide 16,500-nucleotide 18,000; nucleotide 18,500-nucleotide 19,000; nucleotide 21,000-nucleotide 22,500. Especially preferred is the analysis of the methylation status of eight specific CpG dinucleotides, covered in the four sub-sequences of said SEQ ID NO:149 given in SEQ ID NOS:1, 13, 18 and 19. Wherein the method comprises analysis of a gene panel comprising the PITX2 and one or more genes selected from the group consisting ABCA8, CDK6, ERBB2, ONECUT2, PLAU, TBC1D3 and TFF1 and/or regulatory or promoter regions thereof it is preferred that the sequence of said genes is selected from the group consisting of SEQ ID NOS:69 to SEQ ID NO:75, and SEQ ID NO:150 according to TABLE 1.

This methodology presents further improvements over the state of the art in that the method may be applied to any subject, independent of the estrogen and/or progesterone receptor status. Therefore in a preferred embodiment, the subject is not required to have been tested for estrogen or progesterone receptor status.

In further aspects of the invention, the disclosed matter provides novel nucleic acid sequences useful for the analysis of methylation within said gene, other aspects provide novel uses of the gene and the gene product as well as methods, assays and kits directed to providing a prognosis and/or predicting outcome of endocrine treatment of a patient diagnosed with breast cell proliferative disease.

In one embodiment the invention discloses a method for providing the prognosis and/or predicting outcome of endocrine treatment of a patient suffering from a breast cell proliferative disease, by analysis of expression of the gene PITX2 and/or regulatory regions thereof. Preferably said endocrine treatment is an adjuvant endocrine monotherapy. Said method may be enabled by means of any analysis of the expression of the gene, including but not limited to mRNA expression analysis or protein expression analysis or by analysis of its genetic modifications leading to an altered expression (including LOH). However, in the most preferred embodiment of the invention, said expression is determined by means of analysis of the methylation status of CpG sites within the gene PITX2 and its promoter or regulatory elements.

In one embodiment of the method aberrant expression of the gene PITX2 and/or panels thereof may be detected by analysis of loss of heterozygosity of the gene.

In a first step genomic DNA is isolated from a biological sample of the patient's tumor. The isolated DNA is then analyzed for LOH by any means standard in the art including but not limited to amplification of the gene locus or associated microsatellite markers. Said amplification may be carried out by any means standard in the art including polymerase chain reaction (PCR), strand displacement amplification (SDA) and isothermal amplification.

The level of amplificate is then detected by any means known in the art including but not limited to gel electrophoresis and detection by probes (including Real Time PCR). Furthermore the amplificates may be labeled in order to aid said detection. Suitable detectable labels include but are not limited to fluorescence label, radioactive labels and mass labels the suitable use of which shall be described herein.

The detection of a decreased amount of an amplificate corresponding to one of the amplified alleles in a test sample as relative to that of a heterozygous control sample is indicative of LOH.

To detect the levels of mRNA encoding PITX2 and/or panels comprising said gene in a detection system for breast cancer relapse, a sample is obtained from a patient. Said obtaining of a sample is preferably not meant to be retrieving of a sample, as in performing a biopsy, but rather directed to the availability of an isolated biological material representing a specific tissue, relevant for the intended use. The sample can be a tumor tissue sample from the surgically removed tumor, a biopsy sample as taken by a surgeon and provided to the analyst or a sample of blood, plasma, serum or the like. The sample may be treated to extract the nucleic acids contained therein. The resulting nucleic acid from the sample is subjected to gel electrophoresis or other separation techniques. Detection involves contacting the nucleic acids and in particular the mRNA of the sample with a DNA sequence serving as a probe to form hybrid duplexes. The stringency of hybridization is determined by a number of factors during hybridization and during the washing procedure, including temperature, ionic strength, length of time and concentration of formamide. These factors are outlined in, for example, Sambrook et al. (Molecular Cloning: A Laboratory Manual, 2nd ed., 1989). Detection of the resulting duplex is usually accomplished by the use of labeled probes. Alternatively, the probe may be unlabeled, but may be detectable by specific binding with a ligand which is labeled, either directly or indirectly. Suitable labels and methods for labeling probes and ligands are known in the art, and include, for example, radioactive labels which may be incorporated by known methods (e.g., nick translation or kinasing), biotin, fluorescent groups, chemiluminescent groups (e.g., dioxetanes, particularly triggered dioxetanes), enzymes, antibodies, and the like.

To increase the sensitivity of the detection in a sample of mRNA encoding PITX2 and/or panels comprising said gene, the technique of reverse transcription/polymerization chain reaction can be used to amplify cDNA transcribed from mRNA encoding PITX2 and/or panels comprising said gene. The method of reverse transcription/PCR is well known in the art (e.g., see Watson and Fleming, supra).

The reverse transcription/PCR method can be performed as follows: Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and PITX2 and/or panels comprising said gene specific primers (Belyavsky et al, Nucl Acid Res 17:2919-2932, 1989; Krug and Berger, Methods in Enzymology, Academic Press, N.Y., Vol. 152, pp. 316-325, 1987, which are incorporated herein by reference in their entireties)

The present invention may also be described in certain embodiments as a ‘kit’ for use in predicting the likelihood of relapse and/or survival of a breast cancer patient before or after surgical tumor removal with or without adjuvant endocrine monotherapy state through testing of a biological sample. A representative kit may comprise one or more nucleic acid segments as described above that selectively hybridize to PITX2 mRNA and/or mRNA from genes of a panel comprising said PITX2 gene, and a container for each of the one or more nucleic acid segments. In certain embodiments the nucleic acid segments may be combined in a single tube. In further embodiments, the nucleic acid segments may also include a pair of primers for amplifying the target mRNA. Such kits may also include any buffers, solutions, solvents, enzymes, nucleotides, or other components for hybridization, amplification or detection reactions. Preferred kit components include reagents for reverse transcription-PCR, in situ hybridization, Northern analysis and/or RPA.

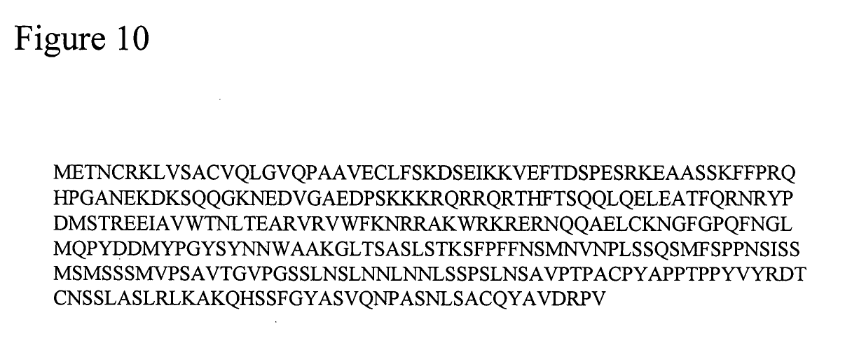

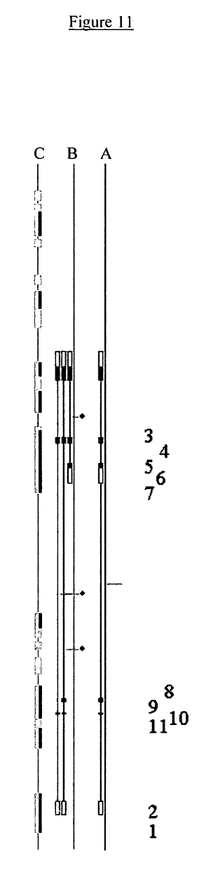

The present invention further provides for methods to detect the presence of the polypeptide(s) of, PITX2 and/or panels comprising said protein, in a sample obtained from a patient. It is preferred that said sequence is essentially the same as the sequence as given in FIG. 10. Any method known in the art for detecting proteins can be used. Such methods include, but are not limited to immunodiffusion, immunoelectrophoresis, immunochemical methods, binder-ligand assays, immunohistochemical techniques, agglutination and complement assays. (e.g., see Basic and Clinical Immunology, Sites and Terr, eds., Appleton & Lange, Norwalk, Conn. pp 217-262, 1991, which is incorporated herein by reference in its entirety). Preferred are binder-ligand immunoassay methods including reacting antibodies with an epitope or epitopes of PITX2 and/or panels thereof and competitively displacing a labeled PITX2 protein and/or panels thereof or derivatives thereof.

Certain embodiments of the present invention comprise the use of antibodies specific to the polypeptide encoded by the gene PITX2 and/or panels comprising said gene. Such antibodies may be useful for providing a prognosis of the likelihood of relapse and/or survival of a breast cancer patient preferably under adjuvant endocrine monotherapy by comparing a patient's levels of PITX2 marker expression and/or the expression of panels comprising PITX2 to expression of the same marker(s) in normal individuals. In certain embodiments the production of monoclonal or polyclonal antibodies can be induced by the use of the PITX2 and/or other polypeptides of the panels as antigene. Such antibodies may in turn be used to detect expressed proteins as markers for prognosis of relapse of a breast cancer patient under adjuvant endocrine monotherapy. The levels of such proteins present in the peripheral blood of a patient may be quantified by conventional methods. Antibody-protein binding may be detected and quantified by a variety of means known in the art, such as labeling with fluorescent or radioactive ligands. The invention further comprises kits for performing the abovementioned procedures, wherein such kits contain antibodies specific for the PITX2 and/or panels thereof polypeptides.

Numerous competitive and non-competitive protein binding immunoassays are well known in the art. Antibodies employed in such assays may be unlabeled, for example as used in agglutination tests, or labeled for use a wide variety of assay methods. Labels that can be used include radionuclides, enzymes, fluorescers, chemiluminescers, enzyme substrates or cofactors, enzyme inhibitors, particles, dyes and the like for use in radioimmunoassay (RIA), enzyme immunoassays, e.g., enzyme-linked immunosorbent assay (ELISA), fluorescent immunoassays and the like. Polyclonal or monoclonal antibodies to PITX2 and/or panels thereof or an epitope thereof can be made for use in immunoassays by any of a number of methods known in the art. One approach for preparing antibodies to a protein is the selection and preparation of an amino acid sequence of all or part of the protein, chemically synthesising the sequence and injecting it into an appropriate animal, usually a rabbit or a mouse (Milstein and Kohler Nature 256:495-497, 1975; Gulfre and Milstein, Methods in Enzymology: Immunochemical Techniques 73:1-46, Langone and Banatis eds., Academic Press, 1981 which are incorporated herein by reference in their entireties). Methods for preparation of PITX2 and/or panels thereof or an epitope thereof include, but are not limited to chemical synthesis, recombinant DNA techniques or isolation from biological samples.

In one aspect the invention provides significant improvements over the state of the art in that it is the first single marker that can be used to predict the likelihood of relapse or of survival of a breast cancer patient under adjuvant endocrine monotherapy.

In the most preferred embodiment of the invention the analysis of expression is carried out by means of methylation analysis. It is further preferred that the methylation state of the CpG dinucleotides within the genomic sequence according to SEQ ID NO:149 and sequences complementary thereto is analyzed. SEQ ID NO:149 discloses the gene PITX2 and its promoter and regulatory elements thereof, wherein said fragment comprises CpG dinucleotides exhibiting a prognosis and/or predicting outcome of endocrine treatment specific methylation pattern. Further preferred is the analysis of the methylation status of CpG positions within the following sections of SEQ ID NO:149: nucleotide 2,700-nucleotide 3,000; nucleotide 3,900-nucleotide 4,200; nucleotide 5,500-nucleotide 8,000; nucleotide 13,500-nucleotide 14,500; nucleotide 16,500-nucleotide 18,000; nucleotide 18,500-nucleotide 19,000; nucleotide 21,000-nucleotide 22,500. Also preferred is the analysis of the sub-sequence of the gene PITX2 as shown in SEQ ID NO:1.

Wherein the method comprises analysis of the expression of a ‘gene panel’ comprising the gene and/or regulatory or promoter regions thereof and one or more genes selected from the group consisting ABCA8, CDK6, ERBB2, ONECUT2, PLAU, TBC1D3 and TFF1 it is almost most preferred that said analysis of expression is carried out by means of methylation analysis. It is particularly preferred that the CpG methylation of the gene panels selected from the group of gene panels consisting:
PITX2, PLAU and TFF1;
PITX2 and PLAU; and
PITX2 and TFF1
   is analyzed.

It is particularly preferred that the methylation of the gene panel consisting PITX2 & TFF1 is determined in order to predict outcome of treatment of patients with an endocrine treatment. It is also particularly preferred that the methylation of the gene panel consisting PITX2 & PLAU is determined in order to provide a prognosis of patients. It is preferred that said patients are analyzed prior to receiving any endocrine treatment.

Hypermethylation of PITX2 and selected other genes as herein and/or sequences thereof are associated with poor prognosis and/or outcome of endocrine treatment of breast cell proliferative disorders, most preferably breast carcinoma.

The methylation pattern of the gene PITX2 and its promoter and regulatory elements have heretofore not been analyzed with regard to prognosis or prediction of outcome of endocrine treatment of a patient diagnosed with a breast cell proliferative disorder. Due to the degeneracy of the genetic code, the sequence as identified in SEQ ID NO:149 should be interpreted so as to include all substantially similar and equivalent sequences upstream of the promoter region of a gene which encodes a polypeptide with the biological activity of that encoded by PITX2.

Most preferably the following method is used to detect methylation within the gene PITX2 and/or regulatory or promoter regions thereof wherein said methylated nucleic acids are present in an excess of background DNA, wherein the background DNA is present in 100 to 1000 times the concentration of the DNA to be detected.

The method for the analysis of methylation comprises contacting a nucleic acid sample obtained from a subject with at least one reagent or a series of reagents, wherein said reagent or series of reagents, distinguishes between methylated and non-methylated CpG dinucleotides within the target nucleic acid.

Preferably, said method comprises the following steps: In the first step, a sample of the tissue to be analyzed is obtained. The source may be any suitable source, preferably, the source of the sample is selected from the group consisting of histological slides, biopsies, paraffin-embedded tissue, bodily fluids, plasma, serum, stool, urine, blood, nipple aspirate and combinations thereof. Preferably, the source is tumor tissue, biopsies, serum, urine, blood or nipple aspirate. The most preferred source, is the tumor sample, surgically removed from the patient or a biopsy sample of said patient.

The DNA is then isolated from the sample. Genomic DNA may be isolated by any means standard in the art, including the use of commercially available kits. Briefly, wherein the DNA of interest is encapsulated in/by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA.

The genomic DNA sample is then treated in such a manner that cytosine bases which are unmethylated at the 5′-position are converted to uracil, thymine, or another base which is dissimilar to cytosine in terms of hybridization behavior. This will be understood as “treatment” or “pre-treatment” herein.

The above described pre-treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior. Enclosing the DNA to be analyzed in an agarose matrix, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996) is one preferred example how to perform said pre-treatment. It is further preferred that the bisulfite treatment is carried out in the presence of a radical scavenger or DNA denaturing agent.

The treated DNA is then analyzed in order to determine the methylation state of the gene PITX2 and/or regulatory regions thereof (prior to the treatment) associated with prognosis and/or outcome of endocrine treatment. In a further embodiment of the method the methylation state of the gene PITX2 and/or regulatory regions thereof and the methylation state of one or more genes selected from the group consisting ABCA8, CDK6, ERBB2, ONECUT2, PLAU, TBC1D3 and TFF1 and/or regulatory or promoter regions thereof is determined. It is particularly preferred that methylation status of a gene panel selected from the group of gene panels consisting PITX2, PLAU & TFF1; PITX2 & PLAU; PITX2 & TFF1 is determined. It is further preferred that the sequences of said genes as described in the accompanying sequence listing (see TABLE 3) are analyzed

In the third step of the method, fragments of the pretreated DNA are amplified. Wherein the source of the DNA is free DNA from serum, or DNA extracted from paraffin it is particularly preferred that the size of the amplificate fragment is between 100 and 200 base pairs in length, and wherein said DNA source is extracted from cellular sources (e.g. tissues, biopsies, cell lines) it is preferred that the amplificate is between 100 and 350 base pairs in length. It is particularly preferred that said amplificates comprise at least one 20 base pair sequence comprising at least three CpG dinucleotides. Said amplification is carried out using sets of primer oligonucleotides according to the present invention, and a preferably heat-stable polymerase. The amplification of several DNA segments can be carried out simultaneously in one and the same reaction vessel, in one embodiment of the method preferably six or more fragments are amplified simultaneously. Typically, the amplification is carried out using a polymerase chain reaction (PCR). The set of primer oligonucleotides includes at least two oligonucleotides whose sequences are each reverse complementary, identical, or hybridize under stringent or highly stringent conditions to an at least 18-base-pair long segment of the base sequences of SEQ ID NOS:2-5, SEQ ID NOS:76 to SEQ ID NO:103 and SEQ ID NOS:151 to SEQ ID NO:158, and sequences complementary thereto.

In a preferred embodiment of the method the primers may be selected from the group consisting to SEQ ID NOS:6 to SEQ ID NO:10.

In an alternate embodiment of the method, the methylation status of preselected CpG positions within the nucleic acid sequences comprising SEQ ID NO:1, SEQ ID NOS:60 to SEQ ID NO:75, SEQ ID NO:149 and SEQ ID NO:150 may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primers pairs contain at least one primer which hybridizes to a bisulfite treated CpG dinucleotide. Therefore, the sequence of said primers comprises at least one CpG, TpG or CpA dinucleotide. MSP primers specific for non-methylated DNA contain a “T” at the 3′ position of the C position in the CpG. Preferably, therefore, the base sequence of said primers is required to comprise a sequence having a length of at least 18 nucleotides which hybridizes to a pretreated nucleic acid sequence according to SEQ ID NOS:2 to SEQ ID NO:5 and SEQ ID NOS: 151, 152, 155 and 156, and sequences complementary thereto, wherein the base sequence of said oligomers comprises at least one CpG, tpG or Cpa dinucleotide. In this embodiment of the method according to the invention it is particularly preferred that the MSP primers comprise between 2 and 4 CpG, tpG or Cpa dinucleotides. It is further preferred that said dinucleotides are located within the 3′ half of the primer, e.g., wherein a primer is 18 bases in length the specified dinucleotides are located within the first 9 bases form the 3′end of the molecule. In addition to the CpG, tpG or Cpa dinucleotides it is further preferred that said primers should further comprise several bisulfite converted bases (i.e. cytosine converted to thymine, or on the hybridizing strand, guanine converted to adenosine). In a further preferred embodiment said primers are designed so as to comprise no more than 2 cytosine or guanine bases.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labeled amplificates have a single positive or negative net charge, allowing for better detectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1:147-57, 1995). The sensitivity with respect to nucleic acid analysis is approximately 100-times less than for peptides, and decreases disproportionally with increasing fragment size. Moreover, for nucleic acids having a multiply negatively charged backbone, the ionisation process via the matrix is considerably less efficient. In MALDI-TOF spectrometry, the selection of the matrix plays an eminently important role. For the desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In a particularly preferred embodiment of the method the amplification of step three is carried out in the presence of at least one species of blocker oligonucleotides. The use of such blocker oligonucleotides has been described by Yu et al., BioTechniques 23:714-720, 1997. The use of blocking oligonucleotides enables the improved specificity of the amplification of a subpopulation of nucleic acids. Blocking probes hybridized to a nucleic acid suppress, or hinder the polymerase mediated amplification of said nucleic acid. In one embodiment of the method blocking oligonucleotides are designed so as to hybridize to background DNA. In a further embodiment of the method said oligonucleotides are designed so as to hinder or suppress the amplification of unmethylated nucleic acids as opposed to methylated nucleic acids or vice versa.

Blocking probe oligonucleotides are hybridized to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘TpG’ at the position in question, as opposed to a ‘CpG.’ In one embodiment of the method the sequence of said blocking oligonucleotides should be identical or complementary to molecule is complementary or identical to a sequence at least 18 base pairs in length selected from the group consisting of SEQ ID NOs: 2 to 5, 151, 152, 155 and 156 preferably comprising one or more CpG, TpG or CpA dinucleotides. In one embodiment of the method the sequence of said oligonucleotides is selected from the group consisting SEQ ID NO:15 and SEQ ID NO:16, and sequences complementary thereto.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivatised at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-termini thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker, a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

In one embodiment of the method, the binding site of the blocking oligonucleotide is identical to, or overlaps with that of the primer and thereby hinders the hybridization of the primer to its binding site. In a further preferred embodiment of the method, two or more such blocking oligonucleotides are used. In a particularly preferred embodiment, the hybridization of one of the blocking oligonucleotides hinders the hybridization of a forward primer, and the hybridization of another of the probe (blocker) oligonucleotides hinders the hybridization of a reverse primer that binds to the amplificate product of said forward primer.

In an alternative embodiment of the method, the blocking oligonucleotide hybridizes to a location between the reverse and forward primer positions of the treated background DNA, thereby hindering the elongation of the primer oligonucleotides.

It is particularly preferred that the blocking oligonucleotides are present in at least 5 times the concentration of the primers.

In the fourth step of the method, the amplificates obtained during the third step of the method are analyzed in order to ascertain the methylation status of the CpG dinucleotides prior to the treatment.

In embodiments where the amplificates are obtained by means of MSP amplification and/or blocking oligonucleotides, the presence or absence of an amplificate is in itself indicative of the methylation state of the CpG positions covered by the primers and or blocking oligonucleotide, according to the base sequences thereof. All possible known molecular biological methods may be used for this detection, including, but not limited to gel electrophoresis, sequencing, liquid chromatography, hybridizations, real time PCR analysis or combinations thereof. This step of the method further acts as a qualitative control of the preceding steps.

In the fourth step of the method amplificates obtained by means of both standard and methylation specific PCR are further analyzed in order to determine the CpG methylation status of the genomic DNA isolated in the first step of the method. This may be carried out by means of hybridization-based methods such as, but not limited to, array technology and probe based technologies as well as by means of techniques such as sequencing and template directed extension.

In one embodiment of the method, the amplificates synthesized in step three are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplificates serve as probes which hybridize to oligonucleotides previously bonded to a solid phase; the non-hybridized fragments are subsequently removed; said oligonucleotides contain at least one base sequence having a length of at least 9 nucleotides which is reverse complementary or identical to a segment of the base sequences specified in the SEQ ID NOD:2 to SEQ ID NO:5 and SEQ ID NOS:151, 152, 155 and 156 and the segment comprises at least one CpG, TpG or CpA dinucleotide. In further embodiments said oligonucleotides contain at least one base sequence having a length of at least 9 nucleotides which is reverse complementary or identical to a segment of the base sequences specified in the SEQ ID NOS:2-5, SEQ ID NOS:151 to SEQ ID NO:158 and SEQ ID NOS:76 to SEQ ID NO:103; and the segment comprises at least one CpG, TpG or CpA dinucleotide.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. In a further embodiment one oligonucleotide exists for the analysis of each CpG dinucleotide within the sequence according to SEQ ID NO:1 and 149, and the equivalent positions within SEQ ID NOS:2 to 5 and SEQ ID NOS:151, 152, 155 and 156. One oligonucleotide exists for the analysis of each CpG dinucleotide within the sequence according to SEQ ID NO:1, SEQ ID NOS:149, 150, and SEQ ID NOS:60 to SEQ ID NO:75, and the equivalent positions within SEQ ID NOS:2-5, SEQ ID NOS:151 to SEQ ID NO:158, and SEQ ID NOS:76 to SEQ ID NO:103. Said oligonucleotides may also be present in the form of peptide nucleic acids. The non-hybridized amplificates are then removed. The hybridized amplificates are detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes that are hybridized to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393). There are two preferred embodiments of utilizing this method. One embodiment, known as the TaqMan™ assay employs a dual-labeled fluorescent oligonucleotide probe. The TaqMan™ PCR reaction employs the use of a non-extendible interrogating oligonucleotide, called a TaqMan™ probe, which is designed to hybridize to a CpG-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. Hybridized probes are displaced and broken down by the polymerase of the amplification reaction thereby leading to an increase in fluorescence. For analysis of methylation within nucleic acids subsequent to bisulfite treatment, it is required that the probe be methylation specific, as described in U.S. Pat. No. 6,331,393, (hereby incorporated by reference in its entirety) also known as the MethyLigh™ assay. The second preferred embodiment of this MethyLight™ technology is the use of dual-probe technology (Lightcycler®), each probe carrying donor or recipient fluorescent moieties, hybridization of two probes in proximity to each other is indicated by an increase or fluorescent amplification primers. Both these techniques may be adapted in a manner suitable for use with bisulfite treated DNA, and moreover for methylation analysis within CpG dinucleotides.

Also any combination of these probes or combinations of these probes with other known probes may be used.

In a further preferred embodiment of the method, the fourth step of the method comprises the use of template-directed oligonucleotide extension, such as MS-SNuPE as described by Gonzalgo & Jones, Nucleic Acids Res. 25:2529-2531, 1997. In said embodiment it is preferred that the methylation specific single nucleotide extension primer (MS-SNuPE primer) is identical or complementary to a sequence at least nine but preferably no more than twenty five nucleotides in length of one or more of the sequences taken from the group of SEQ ID NOS:2 to SEQ ID NO:5 and SEQ ID NOS:151, 152, 155 and 156. However it is preferred to use fluorescently labeled nucleotides, instead of radiolabeled nucleotides.

In yet a further embodiment of the method, the fourth step of the method comprises sequencing and subsequent sequence analysis of the amplificate generated in the third step of the method (Sanger F., et al., Proc Natl Acad Sci USA 74:5463-5467, 1977). In the most preferred embodiment of the methylation analysis method the genomic nucleic acids are isolated and treated according to the first three steps of the method outlined above, namely:
a) obtaining, from a subject, a biological sample having subject genomic DNA;
b) extracting or otherwise isolating the genomic DNA;
c) treating the genomic DNA of b), or a fragment thereof, with one or more reagents to convert cytosine bases that are unmethylated in the 5-position thereof to uracil or to another base that is detectably dissimilar to cytosine in terms of hybridization properties; and wherein
d) amplifying subsequent to treatment in c) is carried out in a methylation specific manner, namely by use of methylation specific primers or blocking oligonucleotides, and further wherein
e) detecting of the amplificates is carried out by means of a real-time detection probe, as described above.

Preferably, where the subsequent amplification of d) is carried out by means of methylation specific primers, as described above, said methylation specific primers comprise a sequence having a length of at least 9 nucleotides which hybridizes to a treated nucleic acid sequence according to one of SEQ ID NOS:2 to SEQ ID NO:5 and SEQ ID NOS:151, 152, 155 and 156, and sequences complementary thereto, wherein the base sequence of said oligomers comprises at least one CpG dinucleotide. Additionally, further methylation specific primers may also be used for the analysis of a gene panel as described above wherein said primers comprise a sequence having a length of at least 9 nucleotides which hybridizes to a treated nucleic acid sequence according to one of SEQ ID NOS:76 to SEQ ID NO:103 and SEQ ID NOS:153, 154, 157 and 158, and sequences complementary thereto, wherein the base sequence of said oligomers comprises at least one CpG dinucleotide.

In an alternative most preferred embodiment of the method, the subsequent amplification of d) is carried out in the presence of blocking oligonucleotides, as described above. It is particularly preferred that said blocking oligonucleotides comprise a sequence having a length of at least 9 nucleotides which hybridizes to a treated nucleic acid sequence according to one of SEQ ID NOS:2 to SEQ ID NO:5 and SEQ ID NOS:151, 152, 155 and 156, and sequences complementary thereto, wherein the base sequence of said oligomers comprises at least one CpG, TpG or CpA dinucleotide.

Additionally, further blocking oligonucleotides may also be used for the analysis of a gene panel as described above wherein said blocking oligonucleotides comprising a sequence having a length of at least 9 nucleotides which hybridizes to a treated nucleic acid sequence according to one of SEQ ID NOS:76 to SEQ ID NO:103 and SEQ ID NOS:153, 154, 157 and 158, and sequences complementary thereto, wherein the base sequence of said oligomers comprises at least one CpG, TpG or CpA dinucleotide.

Step e) of the method, namely the detection of the specific amplificates indicative of the methylation status of one or more CpG positions according to SEQ ID NOS:2-5, SEQ ID NOs:151 to SEQ ID NO:158, and SEQ ID NOS:76 to SEQ ID NO:103, and most preferably SEQ ID NOS:2 to SEQ ID NO:5 and SEQ ID NOS:151, 152, 155 and 156 is carried out by means of real-time detection methods as described above.

Additional embodiments of the invention provide a method for the analysis of the methylation status of the gene PITX2 and/or regulatory regions thereof without the need for pretreatment. Furthermore said method may also be used for the methylation analysis of the gene PITX2 and/or regulatory regions thereof and the methylation state of one or more genes selected from the group consisting ABCA8, CDK6, ERBB2, ONECUT2, PLAU, TBC1D3, TFF1 and/or regulatory or promoter regions thereof is determined. It is particularly preferred that methylation status of a gene panel selected from the group of gene panels consisting PITX2, PLAU and TFF1; PITX2 and PLAU; PITX2 and TFF1 is determined.

In the first step of such additional embodiments, the genomic DNA sample is isolated from tissue or cellular sources. Preferably, such sources include cell lines, histological slides, biopsy tissue, body fluids, or breast tumor tissue embedded in paraffin. Extraction may be by means that are standard to one skilled in the art, including but not limited to the use of detergent lysates, sonification and vortexing with glass beads. Once the nucleic acids have been extracted, the genomic double-stranded DNA is used in the analysis.

In a preferred embodiment, the DNA may be cleaved prior to the treatment, and this may be by any means standard in the state of the art, but preferably with methylation-sensitive restriction endonucleases.

In the second step, the DNA is then digested with one or more methylation sensitive restriction enzymes. The digestion is carried out such that hydrolysis of the DNA at the restriction site is informative of the methylation status of a specific CpG dinucleotide.

In the third step, which is optional but a preferred embodiment, the restriction fragments are amplified. This is preferably carried out using a polymerase chain reaction, and said amplificates may carry suitable detectable labels as discussed above, namely fluorophore labels, radionuclides and mass labels.

In the fourth step the amplificates are detected. The detection may be by any means standard in the art, for example, but not limited to, gel electrophoresis analysis, hybridization analysis, incorporation of detectable tags within the PCR products, DNA array analysis, MALDI or ESI analysis.

In the final step of the method the prognosis and/or predicting outcome of endocrine treatment is determined. Preferably, the correlation of the expression level of the genes with the prognosis and/or predicting outcome of endocrine treatment is done substantially without human intervention. Poor prognosis and/or predicting outcome of endocrine treatment is determined by aberrant levels of mRNA and/or protein, and hypermethylation. It is particularly preferred that said hypermethylation is above average or above median of said disease in said specific setting.

It is particularly preferred that the classification of the sample is carried out by algorithmic means.

In one embodiment machine learning predictors are trained on the methylation patterns at the investigated CpG sites of the samples with known status. A selection of the CpG positions which are discriminative for the machine learning predictor are used in the panel. In a particularly preferred embodiment of the method, both methods are combined; that is, the machine learning classifier is trained only on the selected CpG positions that are significantly differentially methylated between the classes according to the statistical analysis.

The development of algorithmic methods for the classification of a sample based on the methylation status of the CpG positions within the panel are demonstrated in the examples.

The disclosed invention provides treated nucleic acids, derived from genomic SEQ ID NOS:1, SEQ ID NO:149, SEQ ID NO: 150 and SEQ ID NOS:60 to SEQ ID NO:75, wherein the treatment is suitable to convert at least one unmethylated cytosine base of the genomic DNA sequence to uracil or another base that is detectably dissimilar to cytosine in terms of hybridization. The genomic sequences in question may comprise one, or more, consecutive or random methylated CpG positions. Said treatment preferably comprises use of a reagent selected from the group consisting of bisulfite, hydrogen sulfite, disulfite, and combinations thereof. In a preferred embodiment of the invention, the objective comprises analysis of a non-naturally occurring modified nucleic acid comprising a sequence of at least 16 contiguous nucleotide bases in length of a sequence selected from the group consisting of SEQ ID NO: 1, SEQ ID NO:149, SEQ ID NO:150 and SEQ ID NOS:60 to SEQ ID NO:75, wherein said sequence comprises at least one CpG, TpA or CpA dinucleotide and sequences complementary thereto. The sequences of SEQ ID NOS:2-5, SEQ ID NOS:151 to SEQ ID NO:158 and SEQ ID NOS:76 to SEQ ID NO:103 provide non-naturally occurring modified versions of the nucleic acid according to SEQ ID NO:1, SEQ ID NO:149, SEQ ID NO:150 and SEQ ID NOS:60 to SEQ ID NO:75, wherein the modification of each genomic sequence results in the synthesis of a nucleic acid having a sequence that is unique and distinct from said genomic sequence as follows. For each sense strand genomic DNA, e.g., SEQ ID NO: 1, four converted versions are disclosed. A first version wherein “C” to “T,” but “CpG” remains “CpG” (i.e., corresponds to case where, for the genomic sequence, all “C” residues of CpG dinucleotide sequences are methylated and are thus not converted); a second version discloses the complement of the disclosed genomic DNA sequence (i.e. antisense strand), wherein “C” to “T,” but “CpG” remains “CpG” (i.e., corresponds to case where, for all “C” residues of CpG dinucleotide sequences are methylated and are thus not converted). The ‘upmethylated’ converted sequences of SEQ ID NO:1, SEQ ID NO:149, SEQ ID NO:150 and SEQ ID NOS:60 to SEQ ID NO:75 correspond to SEQ ID NOS:2-5, SEQ ID NOS:151 to SEQ ID NO:158 and SEQ ID NOS:76 to SEQ ID NO:103. A third chemically converted version of each genomic sequences is provided, wherein “C” to “T” for all “C” residues, including those of “CpG” dinucleotide sequences (i.e., corresponds to case where, for the genomic sequences, all “C” residues of CpG dinucleotide sequences are unmethylated); a final chemically converted version of each sequence, discloses the complement of the disclosed genomic DNA sequence (i.e. antisense strand), wherein “C” to “T” for all “C” residues, including those of “CpG” dinucleotide sequences (i.e., corresponds to case where, for the complement (antisense strand) of each genomic sequence, all “C” residues of CpG dinucleotide sequences are unmethylated). The ‘downmethylated’ converted sequences of SEQ ID NO:1, SEQ ID NO:149, SEQ ID NO:150 and SEQ ID NOS:60 to SEQ ID NO:75 correspond to SEQ ID NOS:2-5, SEQ ID NOS: 51 to SEQ ID NO:158 and SEQ ID NOS:76 to SEQ ID NO:103.

The invention further discloses oligonucleotide or oligomer for detecting the cytosine methylation state within genomic or pre-treated DNA, according to SEQ ID NO:1, SEQ ID NOS:149 to SEQ ID NO:158 and SEQ ID NOS:60 to SEQ ID NO:103. Said oligonucleotide or oligomer comprising a nucleic acid sequence having a length of at least nine (9) nucleotides which hybridizes, under moderately stringent or stringent conditions (as defined herein above), to a treated nucleic acid sequence according to SEQ ID NOS:2-5, SEQ ID NOS:151 to SEQ ID NO:158 and SEQ ID NOS:76 to SEQ ID NO:103 and/or sequences complementary thereto, or to a genomic sequence according to SEQ ID NO:1, SEQ ID NO:149, SEQ ID NO:150 and SEQ ID NOS:60 to SEQ ID NO:75, and/or sequences complementary thereto.

Thus, the present invention includes nucleic acid molecules (e.g., oligonucleotides and peptide nucleic acid (PNA) molecules (PNA-oligomers)) that hybridize under moderately stringent and/or stringent hybridization conditions to all or a portion of the sequences SEQ ID NOS:2-5, SEQ ID NOS:151 to SEQ ID NO:158 and SEQ ID NOS:76 to SEQ ID NO:103, or to the complements thereof. The hybridizing portion of the hybridizing nucleic acids is typically at least 9, 15, 20, 25, 30 or 35 nucleotides in length. However, longer molecules have inventive utility, and are thus within the scope of the present invention.

Preferably, the hybridizing portion of the inventive hybridizing nucleic acids is at least 95%, or at least 98%, or 100% identical to the sequence, or to a portion thereof of SEQ ID NOS:2-5, SEQ ID NOS:151 to SEQ ID NO:158 and SEQ ID NOS:76 to SEQ ID NO:103, or to the complements thereof.

Hybridizing nucleic acids of the type described herein can be used, for example, as a primer (e.g., a PCR primer), or a diagnostic and/or prognostic probe or primer. Preferably, hybridization of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO:1, SEQ ID NO:149, SEQ ID NO:150 and SEQ ID NOS:60 to SEQ ID NO:75 (such as allelic variants and SNPs), rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridisation reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to, e.g., SEQ ID NO:1, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:
n to (n+(X−1));
where n=1, 2, 3, . . . (Y−(X−1));
where Y equals the length (nucleotides or base pairs) of SEQ ID NO: 1 (9001);
where X equals the common length (in nucleotides) of each oligonucleotide in the set (e.g., X=20 for a set of consecutively overlapping 20-mers); and
where the number (Z) of consecutively overlapping oligomers of length X for a given SEQ ID NO of length Y is equal to Y−(X−1). For example Z=9001−19=8,982 for either sense or antisense sets of SEQ ID NO:1, where X=20.

Preferably, the set is limited to those oligomers that comprise at least one CpG; TpG or CpA dinucleotide.

Examples of inventive 20-mer oligonucleotides include the following set of oligomers (and the antisense set complementary thereto), indicated by polynucleotide positions with reference to SEQ ID NO:1: 1-20, 2-21, 3-22, 4-23, 5-24, . . . and 8,982-9,001.

Preferably, the set is limited to those oligomers that comprise at least one CpG, TpG or CpA dinucleotide.

Likewise, examples of inventive 25-mer oligonucleotides include the following set of oligomers (and the antisense set complementary thereto), indicated by polynucleotide positions with reference to SEQ ID NO:1: 1-25, 2-26, 3-27, 4-28, 5-29, . . . and 8,977-9,001.

Preferably, the set is limited to those oligomers that comprise at least one CpG, TpG or CpA dinucleotide.

The present invention encompasses, for each of SEQ ID NOS:1-5, SEQ ID NOS:149 to SEQ ID NO:158 and SEQ ID NOS:60 to SEQ ID NO:103 (sense and antisense), multiple consecutively overlapping sets of oligonucleotides or modified oligonucleotides of length X, where, e.g., X=9, 10, 17, 20, 22, 23, 25, 27, 30 or 35 nucleotides.

The oligonucleotides or oligomers according to the present invention constitute effective tools useful to ascertain genetic and epigenetic parameters of the genomic sequence corresponding to SEQ ID NO:1, SEQ ID NO:149, SEQ ID NO:150 and SEQ ID NOS:60 to SEQ ID NO:75. Preferred sets of such oligonucleotides or modified oligonucleotides of length X are those consecutively overlapping sets of oligomers corresponding to SEQ ID NOS:1-5, SEQ ID NOS:149 to SEQ ID NO:158 and SEQ ID NOS:60 to SEQ ID NO:103 (and to the complements thereof). Preferably, said oligomers comprise at least one CpG, TpG or CpA dinucleotide.

Particularly preferred oligonucleotides or oligomers according to the present invention are those in which the cytosine of the CpG dinucleotide (or of the corresponding converted TpG or CpA dinculeotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides of the invention can also be modified by chemically linking the oligonucleotide to one or more moieties or conjugates to enhance the activity, stability or detection of the oligonucleotide. Such moieties or conjugates include chromophores, fluorophores, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridisation-triggered cleavage agent, etc.

The oligonucleotide may also comprise at least one art-recognized modified sugar and/or base moiety, or may comprise a modified backbone or non-natural internucleoside linkage.

The oligonucleotides or oligomers according to particular embodiments of the present invention are typically used in ‘sets,’ which contain at least one oligomer for analysis of each of the CpG dinucleotides of genomic sequences SEQ ID NO:1, SEQ ID NO:149, SEQ ID NO:150 and SEQ ID NOS:60 to SEQ ID NO:75, and sequences complementary thereto, or to the corresponding CpG, TpG or CpA dinucleotide within a sequence of the treated nucleic acids according to SEQ ID NOS:2-5, SEQ ID NOS:151 to SEQ ID NOS:158 and SEQ ID NOS:76 to SEQ ID NO:103, and sequences complementary thereto. However, it is anticipated that for economic or other factors it may be preferable to analyze a limited selection of the CpG dinucleotides within said sequences, and the content of the set of oligonucleotides is altered accordingly.

Therefore, in particular embodiments, the present invention provides a set of at least two (2) (oligonucleotides and/or PNA-oligomers) useful for detecting the cytosine methylation state of treated genomic DNA (SEQ ID NOS:2-5, SEQ ID NOS:151 to SEQ ID NO:158 and SEQ ID NOS:76 to SEQ ID NO:103), or in genomic DNA (SEQ ID NO:1, SEQ ID NO:149, SEQ ID NO:150 and SEQ ID NOS:60 to SEQ ID NO:75, and sequences complementary thereto). hese probes enable diagnosis, and/or classification of genetic and epigenetic parameters of lung cell proliferative disorders. The set of oligomers may also be used for detecting single nucleotide polymorphisms (SNPs) in treated genomic DNA (SEQ ID NOS:2-5, SEQ ID NOS:151 to SEQ ID NO:158 and SEQ ID NOS:76 to SEQ ID NO:103), or in genomic DNA (SEQ ID NO:1, SEQ ID NO:149, SEQ ID NO:150 and SEQ ID NOS:60 to SEQ ID NO:75, and sequences complementary thereto).

In preferred embodiments, at least one, and more preferably all members of a set of oligonucleotides is bound to a solid phase.

In further embodiments, the present invention provides a set of at least two (2) oligonucleotides that are used as ‘primer’ oligonucleotides for amplifying DNA sequences of one of SEQ ID NOS:2-5, SEQ ID NOS:151 to SEQ ID NO:158 and SEQ ID NOS:76 to SEQ ID NO:103 and sequences complementary thereto, or segments thereof.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the form of a rectangular or hexagonal lattice. The solid-phase surface may be composed of silicon, glass, polystyrene, aluminium, steel, iron, copper, nickel, silver, or gold. Nitrocellulose as well as plastics such as nylon, which can exist in the form of pellets or also as resin matrices, may also be used. An overview of the prior art in oligomer array manufacturing can be gathered from a special edition of Nature Genetics (Nature Genetics Supplement, Volume 21, January 1999, and from the literature cited therein). Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example, via a confocal microscope. Cy3 and Cy5 dyes, besides many others, are commercially available.

It is also anticipated that the oligonucleotides, or particular sequences thereof, may constitute all or part of an “virtual array” wherein the oligonucleotides, or particular sequences thereof, are used, for example, as ‘specifiers’ as part of, or in combination with a diverse population of unique labeled probes to analyze a complex mixture of analytes. Such a method, for example is described in U.S. 2003/0013091 (U.S. Ser. No. 09/898,743, published 16 Jan. 2003). n such methods, enough labels are generated so that each nucleic acid in the complex mixture (i.e., each analyte) can be uniquely bound by a unique label and thus detected (each label is directly counted, resulting in a digital read-out of each molecular species in the mixture).

The described invention further provides a composition of matter useful for providing a prognosis and/or prediction of outcome of endocrine treatment of breast cancer patients.

Said composition comprising at least one nucleic acid 18 base pairs in length of a segment of the nucleic acid sequence disclosed in SEQ ID NOS:2 to 5 and SEQ ID NOS:151, 152, 155 and 156, and one or more substances taken from the group comprising: magnesium chloride, dNTP, taq polymerase, bovine serum albumen, an oligomer in particular an oligonucleotide or peptide nucleic acid (PNA)-oligomer, said oligomer comprising in each case at least one base sequence having a length of at least 9 nucleotides which is complementary to, or hybridizes under moderately stringent or stringent conditions to a pretreated genomic DNA according to one of the SEQ ID NOS:2 to SEQ ID NO:5 and SEQ ID NOS:151, 152, 155 and 15, and sequences complementary thereto. It is preferred that said composition of matter comprises a buffer solution appropriate for the stabilization of said nucleic acid in an aqueous solution and enabling polymerase based reactions within said solution. Suitable buffers are known in the art and commercially available.

Moreover, an additional aspect of the present invention is a kit comprising, for example: a bisulfite-containing reagent as well as at least one oligonucleotide whose sequences in each case correspond, are complementary, or hybridize under stringent or highly stringent conditions to a 18-base long segment of the sequences SEQ ID NOS:2 to 5 and SEQ ID NOS:151, 152, 155 and 156. Said kit may further comprise at least one oligonucleotide whose sequences in each case correspond, are complementary, or hybridize under stringent or highly stringent conditions to a 18-base long segment of the sequences SEQ ID NOS:2-5, SEQ ID NOS:151-158 and SEQ ID NOS:76 to 103. Said kit may further comprise instructions for carrying out and evaluating the described method. In a further preferred embodiment, said kit may further comprise standard reagents for performing a CpG position-specific methylation analysis, wherein said analysis comprises one or more of the following techniques: MS-SNuPE, MSP, MethyLight®, HeavyMethyl®, COBRA, and nucleic acid sequencing. However, a kit along the lines of the present invention can also contain only part of the aforementioned components.

Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligonucleotide probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Typical reagents (e.g., as might be found in a typical MethyLight™-based kit) for MethyLight™ analysis may include, but are not limited to: PCR primers for specific gene (or methylation-altered DNA sequence or CpG island); TaqMan® probes; optimized PCR buffers and deoxynucleotides; and Taq polymerase.

Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes.

While the present invention has been described with specificity in accordance with certain of its preferred embodiments, the following examples and figures serve only to illustrate the invention and is not intended to limit the invention within the principles and scope of the broadest interpretations and equivalent configurations thereof.

Exemplary SEQ ID NOS:

SEQ ID NOS:6 to 9 provide the nucleic acid sequences of exemplary primers and probes useful to predict the survival of breast cancer patients according to the invention aspect as described in EXAMPLE 4.

SEQ ID NOS:10 to 12 provide the nucleic acid sequences of exemplary primers and probes according to a control gene used in the EXAMPLES 4 and 5.

SEQ ID NO:13 provides a sub-sequence of SEQ ID NO:1, which represents the nucleic acid sequence of the human gene PITX2.

SEQ ID NOS:14 to 17 provide the nucleic acid sequences of those exemplary primers and probes useful to predict the survival of breast cancer patients according to the invention aspects as described in EXAMPLE 5.

SEQ ID NO:21 provides an amino acid sequence of the polypeptide encoded by the gene PITX2. The amino acid sequence of the polypeptide encoded by the gene PITX2 is also illustrated in FIG. 10.

TABLE 1
Genomic sequences and treated variants thereof according to the invention

| | | Sense | Antisense | Sense un- | Antisense |
|---|---|---|---|---|---|
| | | methylated | methylated | methylated | unmethylated |
| Gene | Genomic | converted | converted | converted | converted |
| name | SEQ ID NO: | SEQ ID NO: | SEQ ID NO: | SEQ ID NO: | SEQ ID NO: |
| PITX2 | 1 | 2 | 3 | 4 | 5 |
| PITX2 | 13 | | | | |
| PITX2 | 18 | | | | |
| PITX2 | 19 | | | | |
| ABCA8 | 69 | 76 | 77 | 90 | 91 |
| CDK6 | 70 | 78 | 79 | 92 | 93 |
| ERBB2 | 71 | 80 | 81 | 94 | 95 |
| ONECUT | 72 | 82 | 83 | 96 | 97 |
| PLAU | 73 | 84 | 85 | 98 | 99 |
| TBC1D3 | 74 | 86 | 87 | 100 | 101 |
| TFF1 | 75 | 88 | 89 | 102 | 103 |
| PITX2 | 149 | 151 | 152 | 155 | 156 |
| TFF1 | 150 | 153 | 154 | 157 | 158 |

### EXAMPLES

### Example 1

Study 1. The first study was based on a population of 109 patients, comprising patients of both nodal statuses N0 and N+. All patients were ER+ (estrogen receptor positive). All patients received Tamoxifen monotherapy immediately after surgery or diagnosis. The samples were analyzed using the applicant's chip technology with two chip panels representing 117 candidate genes. For further details see examples in the published patent applications WO 04/035803 and EP 03 090 432.0, which are hereby incorporated by reference. In this study one of the most significant marker gene was PITX2. The methylation status of PITX2, coding for a transcription factor, was statistically significantly correlated with disease-free survival of patients undergoing adjuvant Tamoxifen treatment. This was calculated using the Cox regression model taking into account the nodal status of the patient at the time of diagnosis.

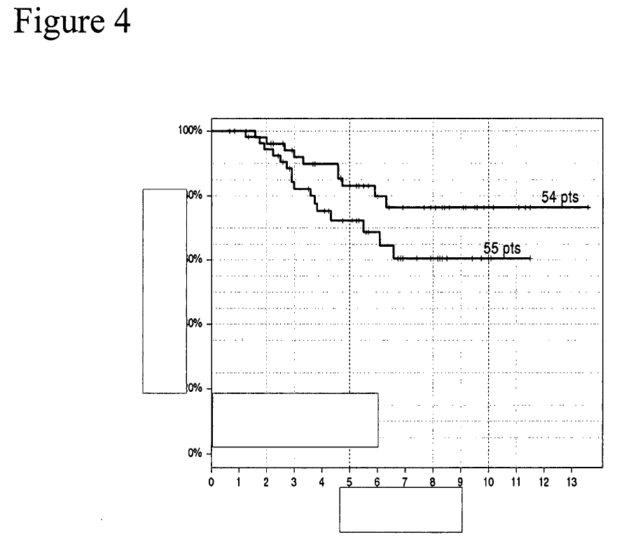

Results. The result from this study, with respect to PITX2, is illustrated in FIG. 4. The X axis shows the metastasis free survival times of the patients in years, and the Y axis shows the proportion of metastasis free survival patients in %. Amongst the 54 patients (upper line) with below median methylation levels have a significantly longer metastasis free survival time than the 55 patients with above median methylation levels (lower line). To illustrate the result, at 10 years after surgery combined with Tamoxifen monotherapy, more than 75% of the patients with below median methylation in PITX2 were still metastasis free, as compared to less than 60% of the patients with above median methylation in PITX2.

As the survival of a breast cancer patient is known to also be correlated to the patient's nodal status, the differentiating power of the marker in this mixed population is expected to be less than in a homogenous population.

Another study was performed to analyze whether the same marker can be identified independently, in a completely different set of patient samples and also to characterize the differential power towards predicting survival for a sub-group of patients, all being N0.

### Example 2

Study 2. The second study was based on samples from 236 patients from 5 different sample providers, wherein all patients were N0 (nodal status negative), and older than 35 years. In all cases surgery was performed before 1998. All patients were ER+ (estrogen receptor positive), and the tumors were graded to be T1-3, G1-3. In this study all patients received Tamoxifen directly after surgery, and the outcome was assessed according to the length of disease-free survival. In order to be as representative as possible for the final target group, the patients and their tumor samples had to fulfil the following criteria:

The range and median follow-up of patients were the following:
Median: 64.5 months
Range: 3 months to 142 months
   (calculated based on patients who were disease-free at end of observation time).

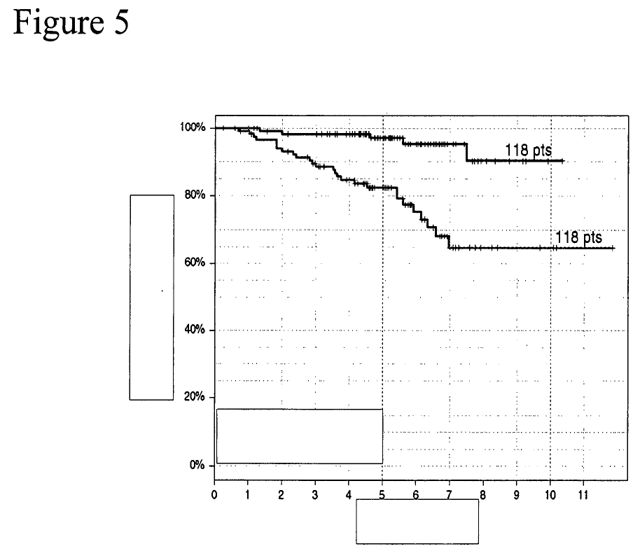

Analysis of the methylation patterns of patient samples treated with Tamoxifen as an adjuvant therapy immediately following surgery (see FIG. 1) is shown in the plots according to FIGS. 5 to 7. For the amplificate, the mean methylation over 4 oligo-pairs for that amplificate was calculated and the population split into groups according to their mean methylation values, wherein one group was composed of individuals with a methylation score higher than the median and a second group composed of individuals with a methylation score lower than the median.

The primer oligonucleotides used to generate the amplificate, that was analyzed in the array experiment were:

| | | |
|---|---|---|
| Array Primer | | |
| PITX2_Q21: | GTAGGGGAGGGAAGTAGATGT | (SEQ ID NO:22) |
| Array Primer | | |
| PITX2_R23: | TCCTCAACTCTACAAACCTAAAA | (SEQ ID NO:23) |

The corresponding genomic region of said amplificate is given in SEQ ID NO:13.

The sequences of the oligonucleotides used in this array experiment were the following:

| | |
|---|---|
| SEQ ID NO xx: | AGTCGGGAGAGCGAAA |
| SEQ ID NO xx: | AGTTGGGAGAGTGAAA |
| SEQ ID NO xx: | AAGAGTCGGGAGTCGGA |
| SEQ ID NO xx: | AAGAGTTGGGAGTTGGA |
| SEQ ID NO xx: | GGTCGAAGAGTCGGGA |
| SEQ ID NO xx: | GGTTGAAGAGTTGGGA |
| SEQ ID NO xx: | ATGTTAGCGGGTCGAA |
| SEQ ID NO xx: | TAGTGGGTTGAAGAGT |

When the data derived from analyzing 6 different CpG sites, located within the preferred amplified region of the PITX2 gene by means of methylation specific detection oligonucleotide hybridization analysis were plotted as Kaplan-Meier estimated metastasis-free survival curves, it can be seen that the differential power of the marker PITX2 increased with selecting for N0 patients. This is shown in FIGS. 5 to 7. The X axis shows the metastasis free survival times of the patients in years, and the Y axis shows the proportion of metastasis free survival patients in %. The lower curve shows the proportion of metastasis free patients in the population with above median methylation levels, and the upper curve shows the proportion of metastasis free patients in the population with below median methylation levels.

For example, as illustrated in FIG. 5, 10 years after surgery only about 65% of the patients of the 118 patients with the higher methylation status are metastasis free, whereas about 90% of the 118 patients with lower methylation status are metastasis free.

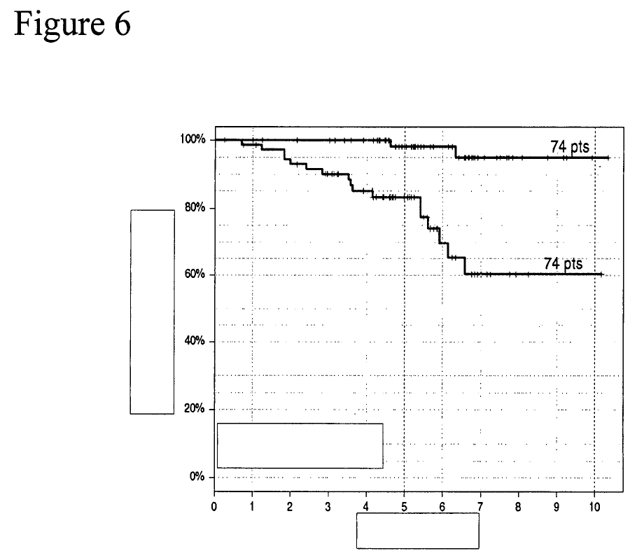

As illustrated in FIG. 6 the analogous Kaplan-Meier analysis for a sub-population of 148 patients, characterized by a tumor at stage G1 or G2 this differential power increases again: 10 years after surgery only about 60% of the 74 patients with the higher methylation status are metastasis free, whereas about 95% of the 74 patients with lower methylation status are metastasis free.

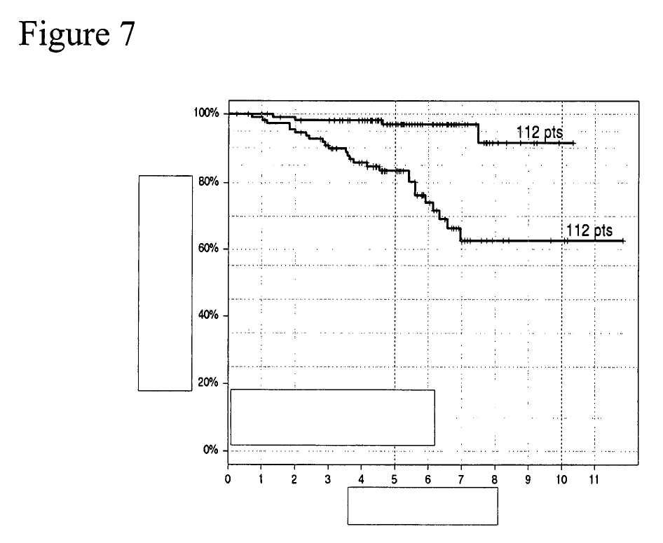

FIG. 7 illustrates how the survival is also correlated to the tumor stage at surgery by showing the analogous Kaplan-Meier analysis for a sub-population of 150 patients, characterized by a tumor stage of T1 or T2: The number of patients with 10 years MFS is about 68% of patients of the 112 with the higher methylation status, whereas about 95% of the 112 patients with lower methylation status are metastasis free.

### Example 3

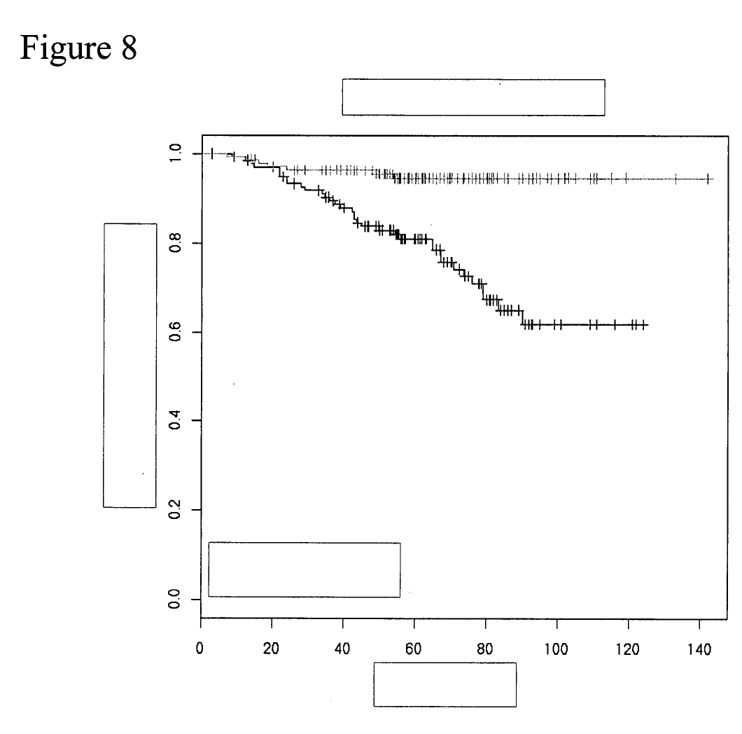

The accuracy of the differentiation between the different groups was further increased by combining multiple oligonucleotides from different genes. As described in the text it was recognized that adding additional informative markers to the analysis could potentially increase the prognostic power of a survival test. Therefore it was calculated how a combination of two methylation specific oligonucleotides each from the genes TBC1D3 and CDK6, and one oligonucleotide from the gene PITX2 would differentiate the groups of good or bad prognosis. The result is shown in FIG. 8 as the according Kaplan-Meier curve.

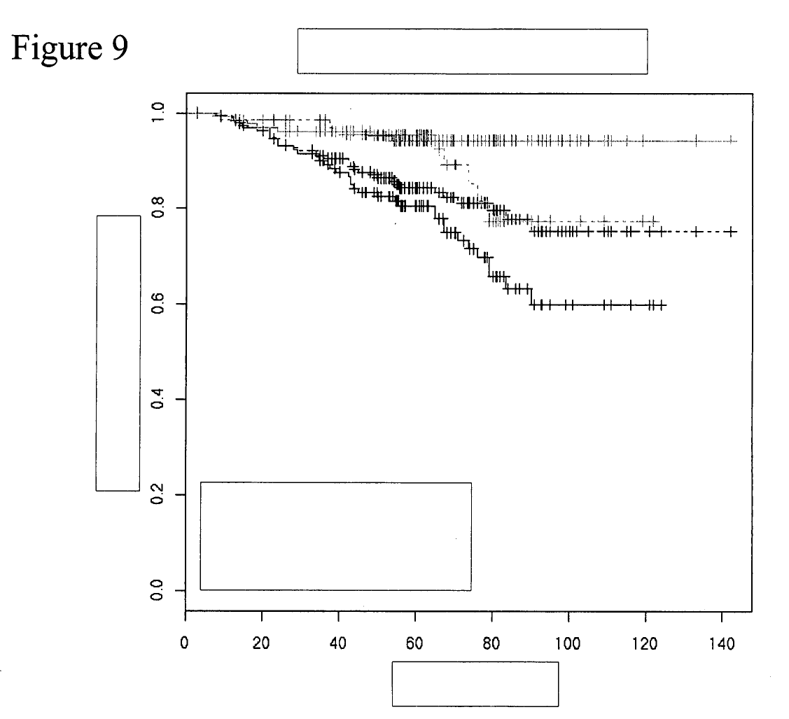

FIG. 9 shows, on top of FIG. 8, the classification of the patients from the sample set by means of the St. Gallen method (the current method of choice for estimating disease free survival), thereby showing the improved effectiveness of methylation analysis over current methods, in particular post 80 months.

### Example 4

Real time quantitative methylation analysis. Genomic DNA was analyzed using the Real Time PCR technique after bisulfite conversion. In this analysis four oligonucleotides were used in each reaction. Two non methylation specific PCR primers were used to amplify a segment of the treated genomic DNA containing a methylation variable oligonucleotide probe binding site. Two oligonucleotide probes competitively hybridize to the binding site, one specific for the methylated version of the binding site, the other specific to the unmethlyated version of the binding site. Accordingly, one of the probes comprises a CpG at the methylation variable position (i.e. anneals to methylated bisulphite treated sites) and the other comprises a TpG at said position (i.e. anneals to unmethylated bisulphite treated sites). Each species of probe is labeled with a 5′ fluorescent reporter dye and a 3′ quencher dye wherein the CpG and TpG oligonucleotides are labeled with different dyes.

The reactions are calibrated by reference to DNA standards of known methylation levels in order to quantify the levels of methylation within the sample. The DNA standards were composed of bisulfite treated phi29 amplified genomic DNA (i.e., unmethlyated), and/or phi29 amplified genomic DNA treated with Sss1 methylase enzyme (thereby methylating each CpG position in the sample), which is then treated with bisulfite solution. Seven different reference standards were used with 0%, (i.e. phi29 amplified genomic DNA only), 5%, 10%, 25%, 50%, 75% and 100% (i.e., phi29 Sss1 treated genomic only).

The amount of sample DNA amplified is quantified by reference to the gene (β-actin (ACTB)) to normalize for input DNA. For standardization the primers and the probe for analysis of the ACTB gene lack CpG dinucleotides so that amplification is possible regardless of methylation levels. As there are no methylation variable positions, only one probe oligonucleotide is required.

The following oligonucleotides were used in the reaction to amplify the control amplificate:

| | |
|---|---|
| Control Primer1: | |
| TGGTGATGGAGGAGGTTTAGTAAGT | (SEQ ID NO:10) |
| Control Primer2: | |
| AACCAATAAAACCTACTCCTCCCTTAA | (SEQ ID NO:11) |
| Control Probe: | |
| 6FAM-ACCACCACCCAACACACAATAACAAACACA- | (SEQ ID NO:12) |
| TAMRA or Dabcyl | |

The nucleic acid sequence of the gene PITX2 is given in (SEQ ID NO:1), after treatment with bisulfite two different strands are generated, and each of the strands is represented twice, once in a prior to treatment methylated version (SEQ ID NOS:2 and 3) and once in the prior to treatment unmethylated form (SEQ ID NOS:4 and 5), which are characterized as containing no cytosine bases (despite of those 5′ adjacent to a guanine and methylated before treatment).

The following primers are used to generate an amplificate within the PITX2 sequence comprising the CpG sites of interest:

Primers for PITX bisulfite amplificate length: 144 bp

| | | |
|---|---|---|
| PITX2: | GTAGGGGAGGGAAGTAGATGTT | (SEQ ID NO:6) |
| PITX2: | TTCTAATCCTCCTTTCCACAATAA | (SEQ ID NO:7) |

The genomic region according to the generated amplificate of 144 bp in length is given in SEQ ID NO:18.

Probes:

| | |
|---|---|
| PITX2cg1: | |
| FAM-AGTCGGAGTCGGGAGAGCGA-Darquencher | (SEQ ID NO:8) |

As an alternative quencher TAMRA was also used in additional experiments:

FAM-AGTCGGAGTCGGGAGAGCGA-TAMRA

| | | |
|---|---|---|
| PITX2tg1: | | |
| YAKIMA | YELLOW-AGTTGGAGTTGGGAGAGTGAAAGG | (SEQ ID NO:9) |
| | AGA Darquencher | |

In additional experiments the following was also used:

VIC- AGTTGGAGTTGGGAGAGTGAAAGGAGA-TAMRA

The extent of methylation at a specific locus was determined by the following formula:
methylation rate=100*I(CG)/(I(CG)+I(TG))
(I=Intensity of the fluorescence of CG-probe or TG-probe)
   PCR components were ordered from Eurogentec:
3 mM MgCl2 buffer, 10× buffer, Hotstart TAQ
Program (45 cycles): 95° C., 10 min; 95° C., 15 sec; 62° C., 1 min

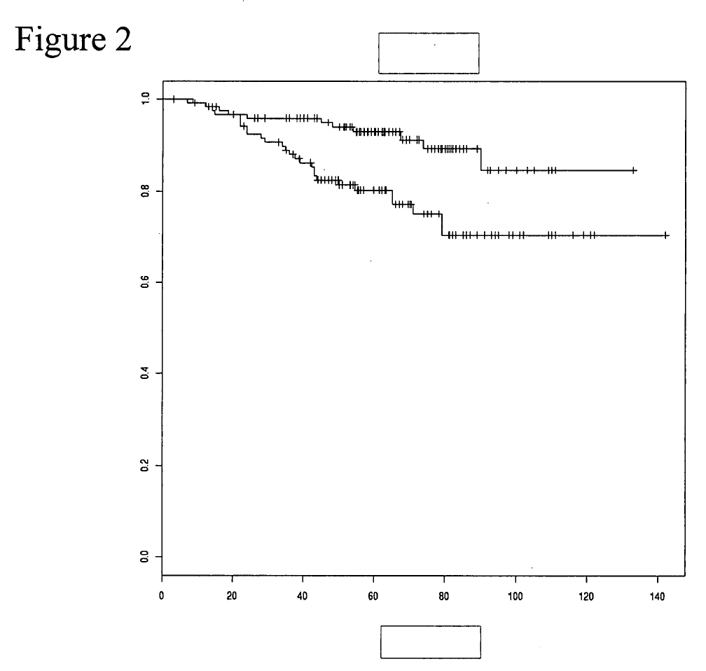

This assay was performed on 236 samples identical to those used in Example 2. The result is shown in FIG. 2. FIG. 2 shows the Kaplan-Meier estimated disease-free survival curves for 3 CpG positions of the PITX2 gene by means of Real-Time methylation specific probe analysis, as described above. The lower curve shows the proportion of disease free patients in the population with above median methylation levels, the upper curve shows the proportion of disease free patients in the population with below median methylation levels. The X axis shows the disease free survival times of the patients in months, and the Y-axis shows the proportion of disease free survival patients. The p-value (probability that the observed distribution occurred by chance) was calculated as 0.0031, thereby confirming the data obtained by means of array analysis.

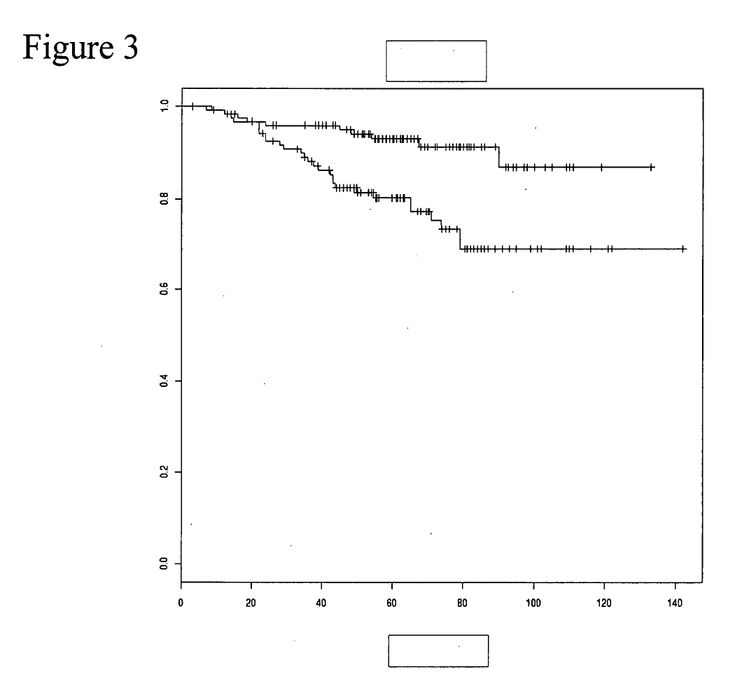

For comparison, FIG. 3 illustrates the result from the array analysis of said gene, according to the chip hybridization experiment described in Example 2, wherein detection oligos were used (for details see EP 03 090 432.0, which is incorporated by reference). The p-value (probability that the observed distribution occurred by chance) was calculated as 0.0011.

### Example 5

Another QM assay was developed, which also performed very well. The following PITX2 specific oligonucleotides were employed to generate an amplificate of 164 bp. The oligonucleotides are specific for three co-methylated CpG positions:

Primers for PITX2 bisulfite amplificate with a length of 162 bp:

| | |
|---|---|
| PITX2: | |
| AACATCTACTTCCCTCCCCTAC | (SEQ ID NO: 14) |
| PITX2: | |
| GTTAGTAGAGATTTTATTAAATTTTATTGTAT | (SEQ ID NO: 15) |

The genomic region according to the generated amplificate of 162 bp in length is given in SEQ ID NO:19.

Probes (from ABI):

| | |
|---|---|
| PITX2-IIcgl: | |
| FAM-TTCGGTTGCGCGGT-MGBNQF | (SEQ ID NO:16) |
| PITX2-IItgl: | |
| VIC-TTTGGTTGTGTGGTTG-MGBNQF | (SEQ ID NO:17) |

The extent of methylation at a specific locus was determined by the following formula:
methylation rate=100*I(CG)/(I(CG)+I(TG))
(I=Intensity of the fluorescence of CG-probe or TG-probe)
PCR components were ordered from Eurogentec: 2.5 mM MgCl2 buffer, 10× buffer, Hotstart TAQ
Program (45 cycles): 95° C., 10 min; 95° C., 15 sec; 60° C., 1 min

### Example 6

LOH Analysis

Patient material. The material to be used in this study, consists of fresh frozen healthy breast tissue, fresh frozen breast tumor tissue from untreated breast cancer patients (follow up over >10 years) and samples from Tamoxifen treated patients (follow up over >10 years from Tamoxifen treatment). Aliquots of DNA from these micro-dissected lesions are used as the source template for PCR-based LOH (Loss of heterozygosity) analysis. All tumor samples were derived from ER+ node negative patients.

LOH analysis. DNA from all tissue samples is subjected to PCR-based LOH analysis using two 4q25-26 markers (D4S1284 and D4S406). These markers define a region on chromosome 4 comprising the gene PITX2 gene said region but being more than 8.5 kbp distant of a region previously shown to undergo LOH in breast carcinomas [Cancer Research 59, 3576-3580, Aug. 1, 1999].

DNA Extraction

Extract DNA from samples using the Wizzard Kit (Promega).

PCR Reaction

See Clin. Cancer Res., 5: 17-23, 1999 for further details.

Analyze each sample by means of single-plex PCR using the following primers:

D4S406

| | |
|---|---|
| Forward primer: | |
| GAAAGGCAGAGTCATAACAGGAAG | (SEQ ID NO:32) |
| Reverse primer: | |
| TAAGGATAGAGTGATTTCCAAGAAAG | (SEQ ID NO:33) |

PCR product size: 205 (bp)
GenBank Accession: Z16728

D4S1284

| | |
|---|---|
| Forward primer: | |
| CTTATCTGACAACAAGCGAGTATG | (SEQ ID NO: 34) |
| Reverse primer: | |
| CAATTATTGTATTGTAGCATCGGAG | (SEQ ID NO: 35) |

PCR product size: 172 (bp)
GenBank Accession: L14168
Synthesize forward primers with either a fluorescent FAM tag (D4S1284) or a fluorescent
TET tag (D4S406) at the 5′ end.
Prepare a suitable quantity of nucleotide mixture according to Table 2.
Aliquot 1 μl of each DNA sample into separate PCR tubes, add 9 μl reaction mixture according to Table 3 and thermal cycle according to the following conditions.
   Thermal Cycling Conditions:
95° C. for 15 min; 39 cycles: 95° C. for 1 min; 55° C. for 0:45; 72° C. for 1:15; and 72° C. for 10 min

Gel electrophoresis. Horizontal ultrathin, high throughput fluorescence-based DNA fragment gel electrophoresis is the preferred technique to separate and analyze the PCR-generated alleles. Combine one microliter of amplified material with 2 μl formamide loading dye (APB) prior to electrophoresis. Add ROX 350 fluorescent size markers (0.7 μl; ABI) to amplified tumor DNA to allow sizing of alleles. Heat samples to 95° C., load on 70 μm, 5% horizontal polyacrylamide gel and electrophorese for 1 h and 15 min at 30 W in 1×TBE.

Data may be collected as commonly known in the art (see for example Clin. Cancer Res., 5: 17-23, 1999).

To determine whether allelic deletion had occurred at individual markers, calculate allelic ratios and express as a percentage of loss of intensity for the treated and untreated tumor samples compared with the corresponding normal samples (D-value) after normalization. When the allelic ratio in the tumor DNA is reduced by greater than 40% (DO.40) from that found in the normal DNA, the sample is denoted as having LOH at that locus.

TABLE 2
Nucleotide Mix

| | |
|---|---|
| 10 | μl dATP, 10 mM |
| 10 | μl dGTP, 10 mM |
| 10 | μl dTTP, 10 mM |
| 2.0 | μl dCTP, 10 mM |
| 288 | μl DEPC-treated H₂O |

TABLE 3
Reaction mixture

| | |
|---|---|
| 1.0 μl | Taq Buffer |
| 0.8 μl | Reduced nucleotide mixture |
| 0.2 μl | Forward primer, 20 μM |
| 0.2 μl | Reverse primer, 20 μM |
| 6.6 μl | DEPC treated H₂0 |
| 0.1 μl | □-32P dCTP |
| 0.1 μl | AmpliTaq Gold Polymerase |
| | Total volume = 9 μl |

### Example 7

Sequencing of gene PITX2. Sequencing of the gene PITX2 was carried out in order to confirm that co-methylation of CpG positions correlated across all exons. For bisulfite sequencing amplification primers were designed to cover 11 sequences within the gene PITX2, see FIG. 11 for further details. Sixteen samples analyzed in Example 4 were utilized for amplicon production. Each sample was treated with sodium bisulfite and sequenced. Sequence data was obtained using ABI 3700 sequencing technology. Obtained sequence traces were normalized and percentage methylation calculated using the Applicant's proprietary bisulphite sequence sequencing trace analysis program (See WO 2004/000463 for further information).

Samples

Eight samples displayed hypermethylation and eight samples displayed hypomethylation in analysis using QM assay as described in example 4.

Amplification

Fragments of interest were amplified using the following conditions

PCR Reaction solution:

| | |
|---|---|
| Taq 5 U/μl | 0.2 |
| dNTPs 25 mM each | 0.2 |
| 10× buffer | 2.5 |
| water | 10.1 |
| primer (6.25 μM) | 2 |
| DNA (1 ng/μl) | 10 |

Cycling Conditions:

15 min 95° C.; 30 s 95° C.; 30 s 58° C.; 1:30 min 72° C. (40 cycles)

TABLE 4
Primers and Amplificates

| Forward primer | Reverse primer | Amplificate | Amplificate |
|---|---|---|---|
| SEQ ID NO: | SEQ ID NO: | SEQ ID NO: | number |
| 36 | 37 | 38 | 1 |
| 39 | 40 | 41 | 2 |
| 42 | 43 | 44 | 3 |
| 45 | 46 | 47 | 4 |
| 48 | 49 | 50 | 5 |
| 51 | 52 | 53 | 6 |
| 54 | 55 | 56 | 7 |
| 57 | 58 | 59 | 8 |
| 60 | 61 | 62 | 9 |
| 63 | 64 | 65 | 10 |
| 66 | 67 | 68 | 11 |

Sequencing

Only g-rich primers were used for sequencing with one exception: Amplificate Number 2 was sequenced using both forward and reverse primer.

ExoSAP-IT Reaction Solution:

4 μl PCR product+2 μl ExoSAP-IT
45 min/37° C. and 15 min/95° C.
   Cycle Sequencing:
1 μl BigDye v.1.1
1 μl water
4 μl Sanger buffer
4 μl dNTP mix (0.025 mM each)
-----
10 μl
+
5 μl Primer (2 pmol/μl)
+
6 μl ExoSAP-IT product
   Cycling
2 min 96° C., 26 cycles a (30 s/96° C., 15 s/55° C., 4 min/60° C.)
   Purification

A 96 well MultiScreen (Millipore) plate was filled with Sephadex G50 (Amersham) using an appropriate admeasure device. 300 μl water were added to each well and incubated 3 h at 4° C. Water was removed by spinning for 5 minutes at 910 g. Cycle sequencing product was loaded to the plate and purified by spinning for 5 min at 910 g. 10 μl of formamide was added to each eluate.

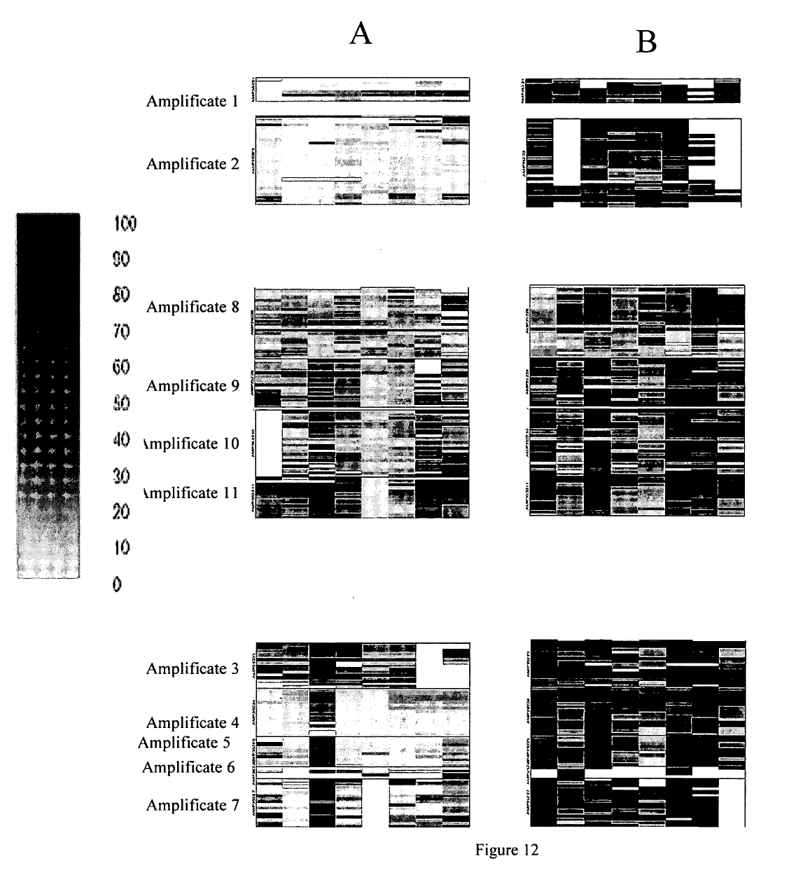

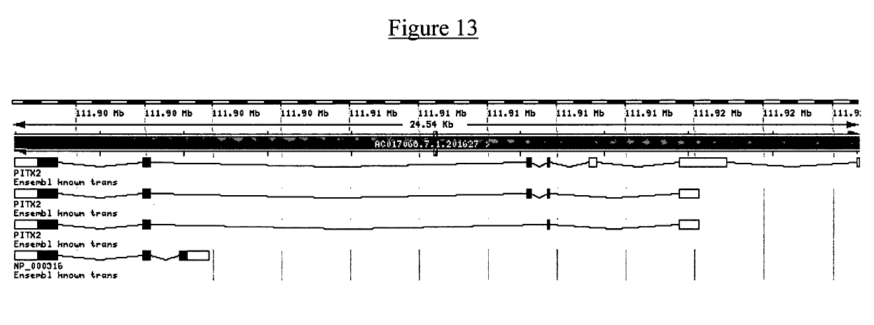

Results. All PCRs yielded a product. FIG. 12 provides matrices produced from bisulfite sequencing data analyzed by the applicant's proprietary software (See WO 2004/000463 for further information). Each column of the matrices of columns ‘A’ and ‘B’ represent the sequencing data for one amplificate. The amplificate number is shown to the left of the matrices. Each row of a matrix represents a single CpG site within the fragment and each column represents an individual DNA sample. The matrices in the column marked ‘A’ showed below median methylation as measured by QM assays (see example 4), the matrices in the column marked ‘B’ showed below median methylation as measured by QM assays.

The bar on the left represents a scale of the percent methylation, with the degree of methylation represented by the shade of each position within the column from black representing 100% methylation to light gray representing 0% methylation. White positions represented a measurement for which no data was available.

Bisulfite sequencing indicated differential methylation of CpG sites between the two selected classes of samples, furthermore co-methylation was observed across the gene. In particular amplificates 4 to 7 showed a high level of differential methylation between the two analyzed groups.

### Example 8

To validate the most promising marker panels from the set of ERBB2, TFF1, PLAU, PITX2, ONECUT, TBC1D3, & ABCA8 Real-Time assays were designed and optimized in order to provide assays of optimum accuracy. The assays were run on a combination of paraffin embedded tissue (hereinafter also referred to as PET) and fresh frozen tissue samples. DNA derived from PET is often of ‘lower quality’ (e.g., higher degree of DNA fragmentation and low DNA yield from samples), thus confirmation of assay results on PET demonstrates the robustness of the assay and increased utility of the marker.

Quantitative methylation assays were designed for the genes ERBB2, TFF1, PLAU, PITX2, ONECUT, TBC1D3, & ABCA8 and tested using a sample set of 415 estrogen receptor positive node negative samples untreated breast cancer patients and 541 estrogen receptor positive node negative samples Tamoxifen treated samples. Approximately 100 of these samples were previously analyzed in the microarray study.

The QM assay (=Quantitative Methylation Assay) is a Real-time PCR based method for quantitative DNA methylation detection. The assay principle is based on non-methylation specific amplification of the target region and a methylation specific detection by competitive hybridization of two different probes specific for the CG or the TG status, respectively. For the present study, TaqMan probes were used that were labeled with two different fluorescence dyes (“FAM” for CG specific probes, “VIC” for TG specific probes) and were further modified by a quencher molecule (“TAMRA” or “Minor Groove Binder/non-fluorescent quencher”).

Evaluation of the QM assay raw data is possible with two different methods:

1. Measuring absolute fluorescence intensities (FI) in the logarithmic phase of amplification
2. Difference in threshold cycles (Ct) of CG and TG specific probe.
   Results of this study were generated by using the Ct method.

In the following series of quantitative methylation assays the amount of sample DNA amplified is quantified by reference to the gene GSTP1 to normalize for input DNA. For standardization, the primers and the probe for analysis of the GSTP1 gene lack CpG dinucleotides so that amplification is possible regardless of methylation levels. As there are no methylation variable positions, only one probe oligonucleotide is required.

Sample Sets

ER+ N0 Untreated Population

To demonstrate that the markers identified have a strong prognostic component, ER+ N0 tumor samples from patients not treated with any adjuvant therapy were analyzed. Markers that are able to show a significant survival difference in this population are considered to be prognostic. All 508 samples of this set were obtained from an academic collaborator as cell nuclei pellets (fresh frozen samples). The sample population can be divided into two subsets: One with 415 randomly selected samples (from both censored and relapsing patients), representing a population with a natural distribution of relapses, and additional 93 samples from relapsing patients only. The latter samples were used for sensitivity/specificity analyses only.

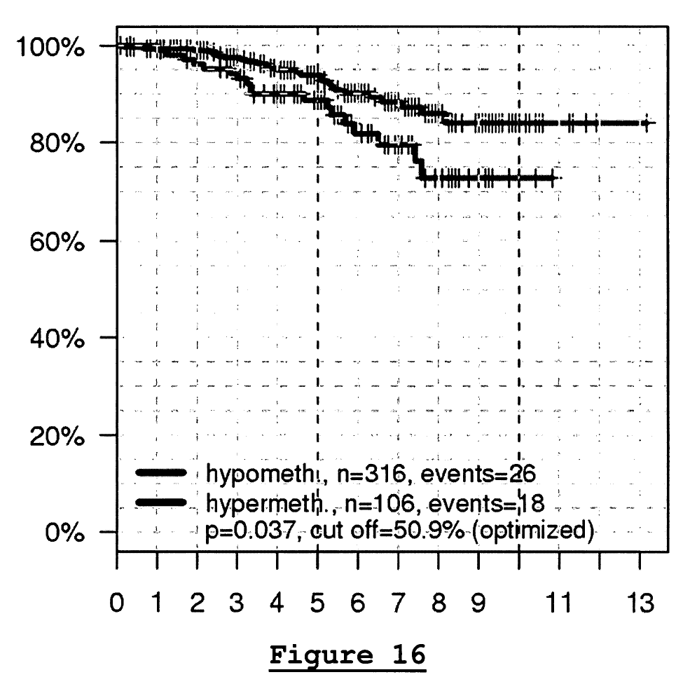

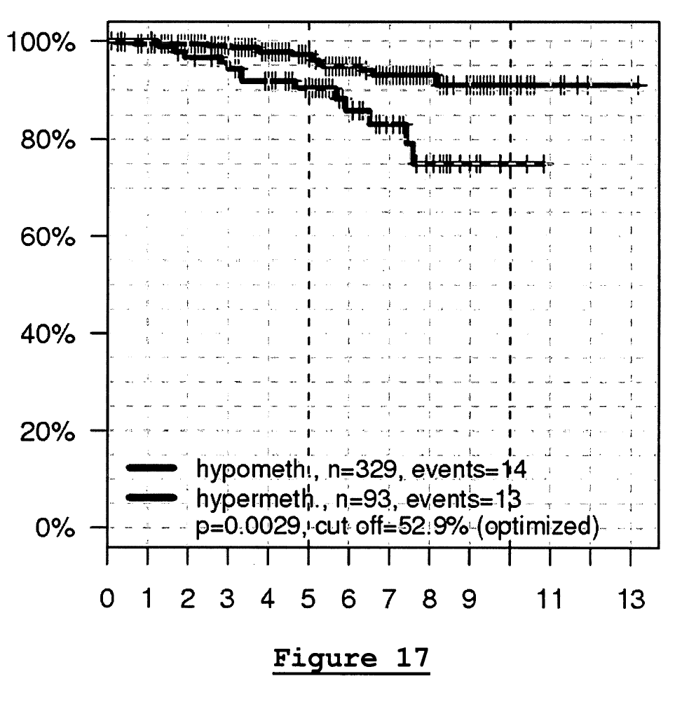

FIG. 16 shows the disease-free survival of the randomly selected population in a Kaplan-Meier plot and FIG. 17 the distribution of follow-up times for the relapsed and censored patients in histograms. TABLE 6 lists the number of events broken down by different kinds of relapse. In summary, the survival of this population is comparable to the expected one from the literature.

ER+ N0 TAM Treated Population

One intended target population of the invention is patients with ER+ N0 tumors that are treated with hormone therapy. To check the performance of the marker candidates in this population, 589 samples from ER+ N0 tumors from patients treated with Tamoxifen were analyzed. All samples were received as Paraffin-embedded tissues (PET). Three to ten 10 μm sections were provided.

In addition, for 89 PET patient samples matching fresh frozen samples from the same tumor were included into the study as controls. As these samples were already used in phase 1, they allowed for two kinds of concordance studies:
Chip versus QM assay
Fresh frozen versus PET samples

Samples of the ER+, N0, TAM treated population were received from eight different providers. Altogether 589 samples were processed, 48 of which had to be excluded from the study due to various reasons (e.g., two samples from same tumor, samples from patients that did not fulfill inclusion criteria etc.).

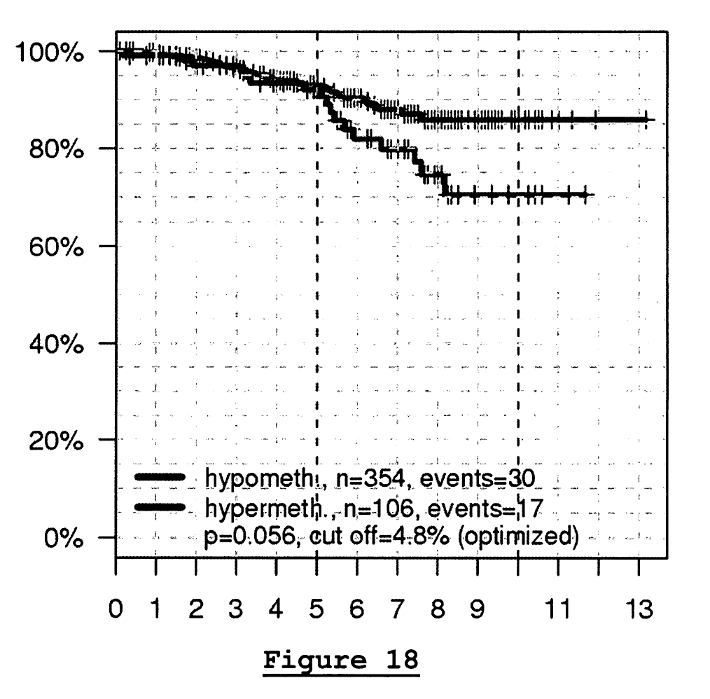

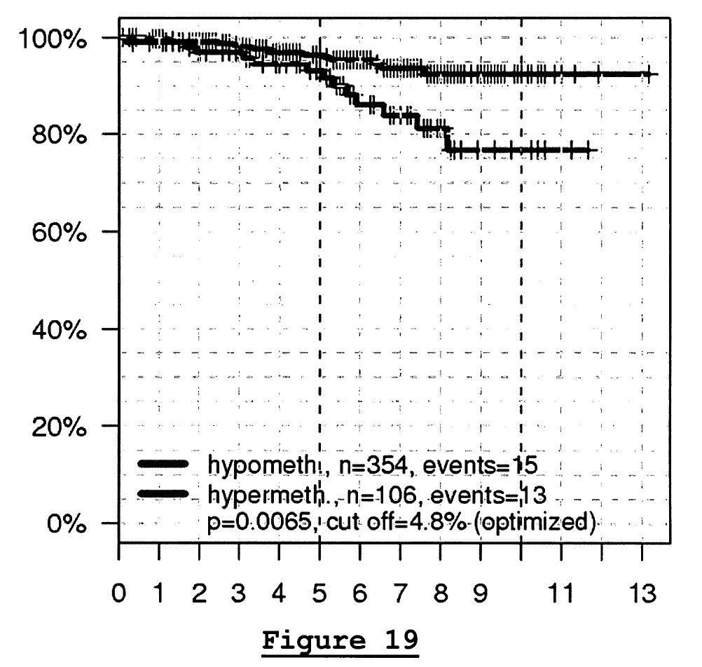

FIG. 18 shows the disease-free survival of the total population in a Kaplan-Meier plot and FIG. 19 the distribution of follow-up times for the relapsed and censored patients in histograms. TABLE 5 lists the number of events broken down by different kinds of relapse. In summary, the survival of this population (82.1% after 10 years) is comparable to the expected one from the literature (79.2%).

DNA Extraction

DNA extraction from Fresh Frozen Samples. From a total of 508 fresh frozen samples available as cell nuclei pellets, genomic DNA was isolated using the QIAamp Kit (Qiagen, Hilden, Germany). The extraction was done according to the Cell Culture protocol using Proteinase K with few modifications.

DNA extraction from PET Samples. 589 provided PET samples were deparaffinated directly in the tube in which they were delivered by the providers. The tissue was then lysed and DNA extracted using the QIAGEN DNeasy Tissue kit.

Bisulfite Treatment

Bisulfite treatment was carried out based on the method disclosed by Olek et al. Nucleic Acids Res. 1996 Dec. 15; 24(24):5064-6, and optimized to the applicant's laboratory workflow.

Quantification Standards. The reactions are calibrated by reference to DNA standards of known methylation levels in order to quantify the levels of methylation within the sample. The DNA standards were composed of bisulfite treated phi29 amplified human genomic DNA (Promega) (i.e. unmethlyated), and/or phi29 amplified genomic DNA treated with Sss1 Methylase enzyme (thereby methylating each CpG position in the sample), which is then treated with bisulfite solution. Seven different reference standards were used with 0%, (i.e., phi29 amplified genomic DNA only), 5%, 10%, 25%, 50%, 75% and 100% (i.e. phi29 Sss1 treated genomic only). 2000 ng batches of human genomic DNA (Promega) were treated with bisulfite. To generate methylated MDA DNA, 13 tubes of 4.5 μg MDA-DNA (700 ng/μl) was treated with Sss1.

Control assay. The GSTP1-C3 assay design makes it suitable for quantitating DNAs from different sources, including fresh/frozen samples, remote samples such as plasma or serum, and DNA obtained from archival specimen such as paraffin embedded material. The following oligonucleotides were used in the reaction to amplify the control amplificate:

| | |
|---|---|
| Control Primer1: | |
| GGAGTGGAGGAAATTGAGAT | (SEQ ID NO:104) |
| Control Primer2: | |
| CCACACAACAAATACTCAAAAC | (SEQ ID NO: 105) |
| Control Probe: | |
| FAM-TGGGTGTTTGTAATTTTTGTTTTGTGTTA- | (SEQ ID NO:106) |
| GGTT-TAMRA | |

| | |
|---|---|
| Cycle program (40 cycles): | 95° C., 10 min |
| | 95° C., 15 sec |
| | 58° C., 1 min |

Assay Design and Reaction Conditions
Two assays were developed for the analysis of the gene PITX2(SEQ ID NO:23)

Assay 1:

| | |
|---|---|
| Primers: | |
| GTAGGGGAGGGAAGTAGATGTT | (SEQ ID NO:107) |
| TTCTAATCCTCCTTTCCACAATAA | (SEQ ID NO:108) |
| Probes: | |
| FAM-AGTCGGAGTCGGGAGAGCGA-TAMRA | (SEQ ID NO:109) |
| VIC-AGTTGGAGTTGGGAGAGTGAAAGGAG | (SEQ ID NO:110) |
| A -TAMRA | |

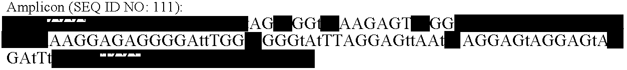

Length of fragment: 143 bp

Positions of primers, probes and CpG dinucleotides ar highlighted.

PCR components (supplied by Eurogentec): 3 mM MgCl2 buffer, 10× buffer, Hotstart TAQ, 200 μM dNTP, 625 nM each primer, 200 nM each probe

| | |
|---|---|
| Cycle program (45 cycles): | 95° C., 10 min |
| | 95° C., 15 sec |
| | 62° C., 1 min |

Assay 2:

| | |
|---|---|
| Primers: | |
| AACATCTACTTCCCTCCCCTAC | (SEQ ID NO:112) |
| GTTAGTAGAGATTTTATTAAATTTTATTGTAT | (SEQ ID NO:113) |
| Probes: | |
| FAM-TTCGGTTGCGCGGT-MGBNQF | (SEQ ID NO:114) |
| VIC-TTTGGTTGTGTGGTTG- MGBNQF | (SEQ ID NO:115) |

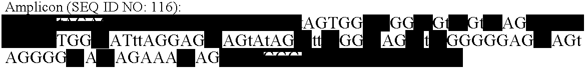

Length of fragment: 164 bp

The positions of probes, primers and CpG positions are highlighted.

The probes cover three co-methylated CpG positions.

PCR components (supplied by Eurogentec): 2.5 mM MgCl2 buffer, 10× buffer, Hotstart TAQ, 200 μM dNTP, 625 nM each primer, 200 nM each probe

| | |
|---|---|
| Program (45 cycles): | 95° C., 10 min |
| | 95° C., 15 sec |
| | 60° C., 1 min |

The extent of methylation at a specific locus was determined by the following formulas:
Using absolute fluorescence intensity: methylation rate=100*I(CG)/(I(CG)+I(TG)) (I=Intensity of the fluorescence of CG-probe or TG-probe)
Using threshold cycle Ct: methylation rate=100*CG/(CG+TG)=100/(1+TG/CG)=100/(1+2ˆdelta(ct))
(assuming PCR efficiency E=2; delta (Ct)=Ct (methylated)−Ct (unmethylated))

Gene PLAU

| | |
|---|---|
| Primer: | |
| GTTAGGTGTATGGGAGGAAGTA | (SEQ ID NO:117) |
| TCCCTCCCCTATCTTACAA | (SEQ ID NO:118) |
| Probes: | |
| FAM-ACCCGAACCCCGCGTACTTC-TAMRA | (SEQ ID NO:119) |
| VIC-ACCCAAACCCCACATACTTCCACA-TAMRA | (SEQ ID NO:120) |

Amplicon (SEQ ID NO:121): 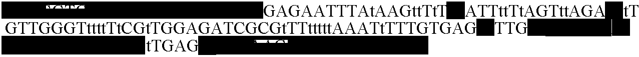
Length of fragment: 166 bp

The positions of probes, primers and CpG positions are highlighted.

PCR components were supplied by Eurogentec: 2.5 mM MgCl2 buffer, 10× buffer, Hotstart TAQ, 200 μM dNTP, 625 nM each primer, 200 nM each probe

| | |
|---|---|
| Program (45 cycles): | 95° C., 10 min |
| | 95° C., 15 sec |
| | 60° C., 1 min |

Gene ONECUT2

| | |
|---|---|
| Primer: | |
| GTAGGAAGAGGTGTTGAGAAATTAA | (SEQ ID NO:122) |
| CCACACAAAAAATTTCTATACTCCT | (SEQ ID NO:123) |
| Probes: | |
| FAM- ACGGGTAGAGGCGCGGGT -TAMRA | (SEQ ID NO:124) |
| VIC- ATGGGTAGAGGTGTGGGTTATATTGTTTT | (SEQ ID NO:125) |
| G-TAMRA | |

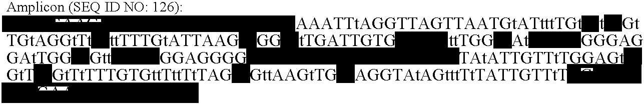 [0414] Length of fragment: 266 bp

The positions of probes, primers and CpG positions are highlighted.

PCR components were supplied by Eurogentec: 3 mM MgCl2 buffer, 10× buffer, Hotstart TAQ, 200 μM dNTP, 625 nM each primer, 200 nM each probe

| | |
|---|---|
| Program (45 cycles): | 95° C., 10 min |
| | 95° C., 15 sec |
| | 60° C., 1 min |

Gene ABCA8

| | |
|---|---|
| Primer: | |
| GTGAGGTATTGGATTTAGTTTATTTG | (SEQ ID NO:127) |
| CCCTAAATCTCATCCTAAAAACAC | (SEQ ID NO:128) |
| Probes: | |
| FAM- TGAGGTTTCGGTTTTTAACGGTGG | (SEQ ID NO:129) |
| -TAMRA | |
| VIC- TGAGGTTTTGGTTTTTAATGGTGGGAT | (SEQ ID NO:130) |
| -TAMRA | |

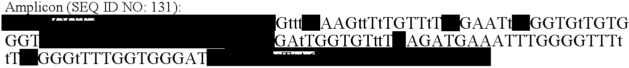 [0419] Length of fragment: 168 bp

The positions of probes, primers and CpG positions are highlighted.

PCR components were supplied by Eurogentec: 3 mM MgCl2 buffer, 10× buffer, Hotstart TAQ, 200 μM dNTP, 625 nM each primer, 200 nM each probe

| | |
|---|---|
| Program (45 cycles): | 95° C., 10 min |
| | 95° C., 15 sec |
| | 62° C., 1 min |

Gene ERBB2

| | |
|---|---|
| Primer: | |
| GGAGGGGGTAGAGTTATTAGTTTT | (SEQ ID NO:134) |
| ACTCCCAACTTCACTTTCTCC | (SEQ ID NO:135) |
| Probes: | |
| FAM- TAATTTAGGCGTTTCGGCGTTAGG | (SEQ ID NO:136) |
| -TAMRA | |
| VIC- TAATTTAGGTGTTTGGTGTTAGGAGGG | (SEQ ID NO:137) |
| A -TAMRA | |

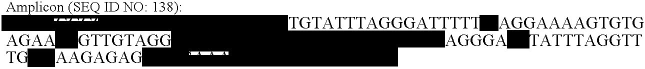 [0424] Length of fragment: 144 bp

The positions of probes, primers and CpG positions are highlighted.

PCR components were supplied by Eurogentec: 2.5 mM MgCl2 buffer, 10× buffer, Hotstart TAQ, 200 μM dNTP, 625 nM each primer, 200 nM each probe

| | |
|---|---|
| Program (45 cycles): | 95° C., 10 min |
| | 95° C., 15 sec |
| | 62° C., 1 min |

Gene TFF1

| | |
|---|---|
| Primer: | |
| AGTTGGTGATGTTGATTAGAGTT | (SEQ ID NO:139) |
| CCCTCCCAATATACAAATAAAAACTA | (SEQ ID NO:140) |
| Probes: | |
| FAM- ACACCGTTCGTAAAA-MGBNFQ | (SEQ ID NO:141) |
| VIC- ACACCATTCATAAAAT-MGBNFQ | (SEQ ID NO:142) |

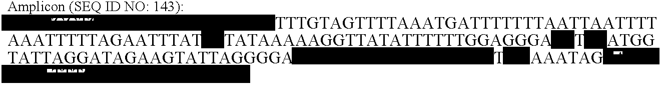 [0429] Length of fragment: 189 bp

The positions of probes, primers and CpG positions are highlighted.

PCR components were supplied by Eurogentec: 2.5 mM MgCl2 buffer, 10× buffer, Hotstart TAQ, 200 μM dNTP, 625 nM each primer, 200 nM each probe

| | |
|---|---|
| Program (45 cycles): | 95° C., 10 min |
| | 95° C., 15 sec |
| | 60° C., 1 min |

Gene TBC1D3

| | |
|---|---|
| Primer: | |
| TTTTTAGTTGGTTTTTATTAGGGTTT | (SEQ ID NO:144) |
| CCAACATATCCACCCACTTACT | (SEQ ID NO:145) |
| Probes: | |
| FAM- TTTCGACTAATCTCCCGCCGA-TAMRA | (SEQ ID NO:146) |
| VIC- TTTCAACTAATCTCCCACCAATTTACT | (SEQ ID NO:147) |
| ATCA-TAMRA | |

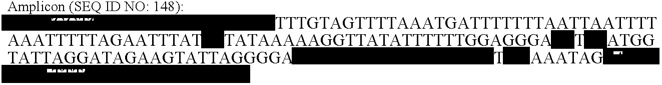 [0434] Length of fragment: 142 bp

The positions of probes, primers and CpG positions are highlighted.
PCR components were supplied by Eurogentec: 4.5 mM MgCl2 buffer, 10× buffer, Hotstart TAQ, 200 μM dNTP, 625 nM each primer, 200 nM each probe
Program (45 cycles): 95° C., 10 min; 95° C., 15 sec; 60° C., 1 min

Each of the designed assays was tested on the following sets of samples:
Tamoxifen treated patients who relapsed during treatment (all relapses).
Tamoxifen treated patients who relapsed during treatment with distant metastases only.
Non-Tamoxifen treated patients who relapsed during treatment (all relapses).
Non-Tamoxifen treated patients who relapsed during treatment with distant metastases only.
   Raw Data Processing

All analyses were based on CT evaluation (evaluation using fluorescence intensities are available upon request). Assuming optimal real-time PCR conditions in the exponential amplification phase, the concentration of methylated DNA (Cₘₑₜₕ) can be determined by

$${C}_{meth} = \frac{100}{1 + {2}^{( {CT}_{CG} - {CT}_{TG} )}} [ \% ] ,$$

where
CT_{CG} denotes the threshold cycle of the CG reporter (FAM channel); and
CT_{TG} denotes the threshold cycle of the TG reporter (VIC channel).

The thresholds for the cycles were determined by human experts after a visual inspection of the Amplification Plots [ABI PRISM 7900 HT Sequence Detection System User Guide]. The values for the cycles (CT_{CG} and CT_{TG}) were calculated with these thresholds by the ABI 7900 software. Whenever the amplification curve did not exceed the threshold, the value of the cycle was set to the maximum cycle (i.e., 50).

Statistical Methods

Cox Regression. The relation between disease-free survival times (DFS) (or metastasis free survival, MFS) and covariates are modeled using Cox Proportional Hazard models (Cox and Oates, 1984; Harrel, 2001). The hazard, i.e. the instantaneous risk of a relapse, is modeled as
h(t|x)=h₀(t)·exp(βx) (3)
and
h(t|x₁, . . . xₖ)=h₀(t)·exp(β₁x₁+ . . . +βₖxₖ) (4)
for univariate and multiple regression analyses, respectively, where t is the time measured in months after surgery, h₀(t) is the baseline hazard, x is the vector of covariates (e.g., measurements of the assays) and β is the vector of regression coefficients (parameters of the model). β will be estimated by maximizing the partial likelihood of the Cox proportional hazard model Likelihood ratio tests are performed to test whether methylation is related to the hazard. The difference between 2Log (Likelihood) of full model and null-model is approximately x²-distributed with k degrees of freedom under the null hypotheses β₁= . . . =βₖ=0.

The assumption of proportional hazards were checked by scaled Schoenfeld residuals (Thernau et al., 2000).

For the calculation, analysis and diagnostic of the Cox Proportional Hazard Model the R functions coxph, coxph.zph of the “survival” package were used.

Stepwise Regression Analysis

For multivariate Cox regression models a stepwise procedure (Venables et al., 1999; Harrel, 2001) was used in order to find sub-models including only relevant variables. Two effects are usually achieved by these procedures:
Variables (methylation rates) that are basically unrelated to the dependent variable (DFS/MFS) are excluded as they do not add relevant information to the model.
Out of a set of highly correlated variables, only the one with the best relation to the dependent variable is retained.
   Inclusion of both types of variables can lead to numerical instabilities and a loss of power. Moreover, the predictory performance can be low due to overfitting.

The applied algorithm aims at minimizing the Akaike information criterion (AIC) which is defined as
AIC=2□maximized log-likelihood+2□#parameters.

The AIC is related to the predictory performance of a model, smaller values promise better performance. Whereas the inclusion of additional variables always improves the model fit and thus increases the likelihood, the second term penalizes the estimation of additional parameters. The best model will present a compromise model with good fit and usually a small or moderate number of variables.

Stepwise regression calculation with AIC was done with the R function “step”.

Kaplan-Meier Survival Curves and Log-Rank Tests

Survival curves are estimated from DFS/MFS data using the Kaplan-Meier method (Kaplan and Meier, 1958). Log-rank tests were used to test for differences of two survival curves, e.g. survival in hyper- vs. hypomethylated groups. For a description of this test see (Cox and Oates, 1984).

For the Kaplan Meier Analysis the functions “survfit” and “survdiff” of the “survival” package were used.

Independence of Markers from Other Covariates

To check whether our marker panel gives additional and independent information, other relevant clinical factors were included in the cox proportional hazard model and the p-values for the weights for every factor were calculated (Wald-Test) (Thernau et al., 2000). For the analysis of additional factors in the Cox Proportional Hazard model, the R function “coxph” was used.

Correlation Analysis

Pearson and Spearman correlation coefficients are calculated to estimate the concordance between measurements (e.g., methylation in matched fresh frozen and PET samples).

Density Estimation

For numerical variables, kernel density estimation was performed with a gaussian kernel and variable bandwidth. The bandwidth is determined using Silverman's “rule-of-thumb” (Silverman, 1986). For the calculation of the densities the R function “density” was used.

Analysis of Sensitivity and Specificity

For the analysis of sensitivity and specificity of single assays and marker panels ROCs were calculated. The calculation of the ROCs was done with two methods: The first method is to calculate sensitivity and specificity for a given threshold for the time T_{Threshold}. With that threshold, true positives, false positives, true negatives and false negatives were defined and the values for sensitivity and specificity were calculated for different cutoffs of the model. Patients censored before T_{Threshold} were excluded. The ROCs were calculated for different times T_{Threshold} (3 year, 4 years, . . . , 10 years). The second method is to calculate sensitivity and specificity by using the Bayes-formula based on the Kaplan-Meier estimates (Heagerty et al., 2000) for the survival probabilities in the marker positive and marker negative groups for a given time T_{Threshold}. The ROCs were calculated for different times T_{Threshold} (3 year, 4 years, . . . 10 years).

k-Fold Crossvalidation

For the analysis of model selection and model robustness k-fold crossvalidation (Hastie et al., 2001) was used. The set of observation was split in k chunks by random. In turn, every chunk was used as a test set and the remaining k−1 chunks were used as training set. This procedure was repeated n times.

Population Charts

For the description of the relation between censoring and a covariate Population Charts (Mocks et al., 2002) were used. The baseline of the covariate was calculated including all observations with event. For a given time t, the mean (in case of real variables like age) or the fraction (in case of categorical variables) for all censored patients in the risk set at time t was calculated and added to the baseline value.

Technical Performance

Comparison of Assay Replicates

Each marker was measured in at least three replicates, variability between assay replicates was observed to be higher for PET than for fresh frozen samples.

Concordance Study Fresh Frozen Versus PET Samples

Markers analyzed in this study (Example 2) were initially identified on a chip platform (EXAMPLE 1) using fresh frozen samples. The ER+ N0 untreated population was also analyzed on fresh frozen samples in EXAMPLE 2. A concordance study should demonstrate that measured methylation ratios are comparable for fresh frozen and PET samples. For this purpose, 89 fresh frozen samples from three different providers already used in the chip study were processed again in parallel with a matching PET sample originating from the same tumor.

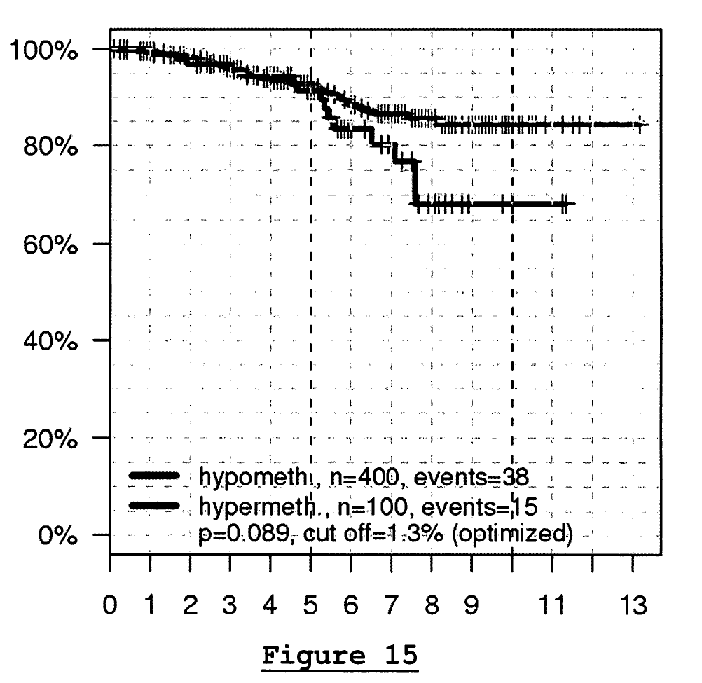

FIG. 15 shows such a concordance study for marker candidate PITX2 assay 1 as a scatter plot between fresh frozen and PET samples (using the QM assay). The association between the paired samples is 0.81 (Spearman's rho). This analysis is based on n=89 samples.

Results

Evaluation of Single Markers. Each of the eight established QM assays was used to measure the 508 samples from the N0, ER+ untreated patient population (random selection and additional relapses) in three replicates. After filtering of measuring points not fulfilling quality criteria and performing a Cox analyses, Kaplan-Meier survival curves and ROC curves for each single marker were generated.

Two different clinical endpoints were used for analyses:
Disease-free survival, i.e. using all kinds of relapses (distant metastasis, loco-regional relapses, relapses at contralateral breast) as event.
Metastasis-free survival, i.e. treating only distant metastasis as an event.

For analyzing the ER+, N0, TAM treated population, five marker candidates were analyzed on 541 samples from the N0, ER+ untreated patient population. Assays were measured in three replicates. Three assays that were measured on the untreated population (PITX-2, ONECUT, and ABCA8) were not measured due to the limited material that was available for the TAM treated population. These assays were rejected either because they performed bad in the untreated population (ONECUT and ABCA8) or in case of PITX2-II it performed significantly worse than the other assay of this marker (PITX2-I). After filtering of measuring points not fulfilling quality criteria Kaplan-Meier survival curves and ROC curves for each single marker were generated.

Two different clinical endpoints were used:
Disease-free survival, i.e., using all kinds of relapses (distant metastasis, locoregional relapses, relapses at contralateral breast) as event.
Metastasis-free survival, i.e. treating only distant metastasis as an event.

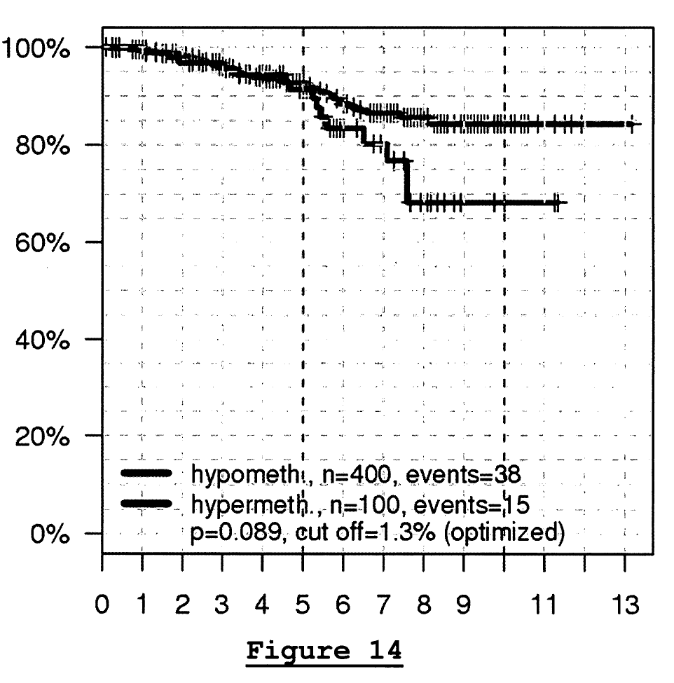

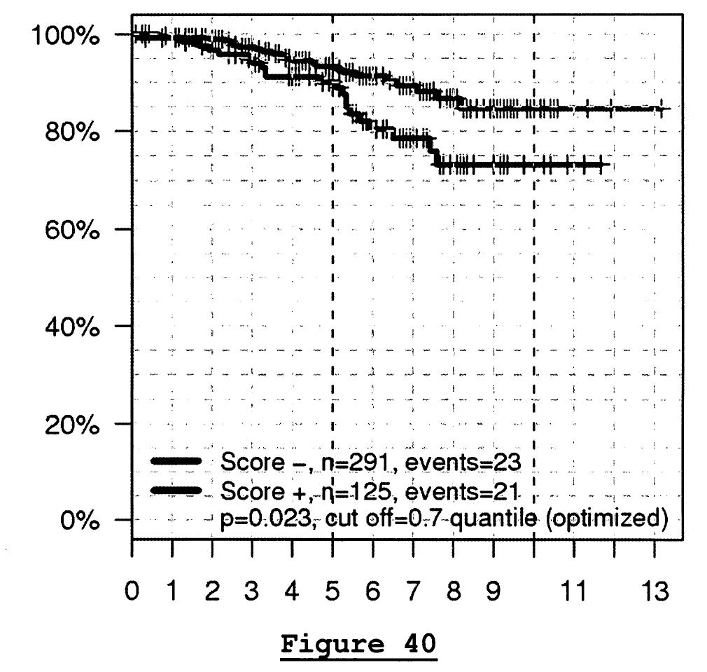

The Kaplan-Meier estimated disease-free survival or metastasis-free survival curves of each single assay are shown in FIGS. 14 to 39, and combinations of assays are shown in FIGS. 40 to 55. The X axis shows the disease free survival times of the patients in years, and the Y-axis shows the proportion of patients with disease free survival. The black plot shows the proportion of disease free patients in the population with above an optimized cut off point's methylation levels, the gray plot shows the proportion of disease free patients in the population with below an optimized cut off point's methylation levels.

The following p-values (probability that the observed distribution occurred by chance) were calculated when the cut off was optimized. For cut-off optimization, the quantiles of both groups were shifted between 0.2 and 0.8 and the p-value for the separation of the curves was calculated for each quantile. The quantile with the lowest p-value was then the best cutoff. Percentage values refer to the methylation ratios at the cut-off point.

### Single Gene Assays

Tamoxifen Treated

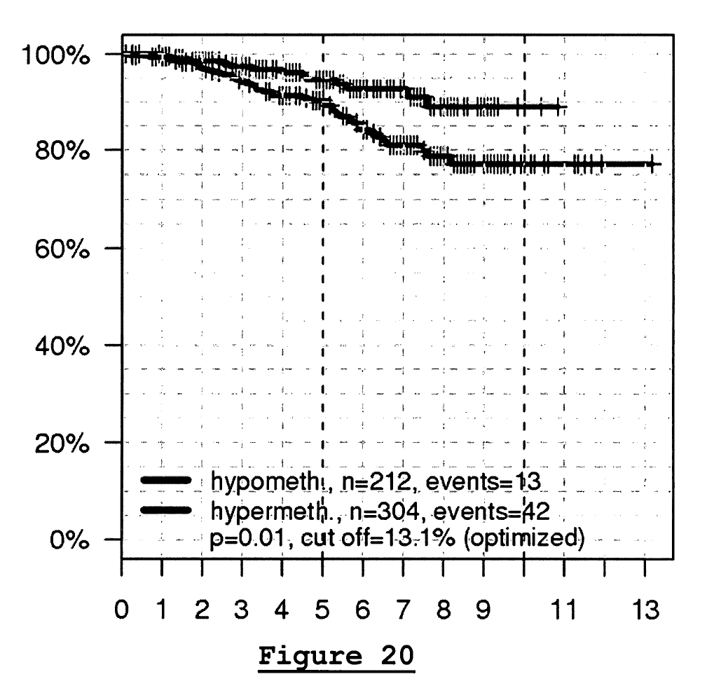

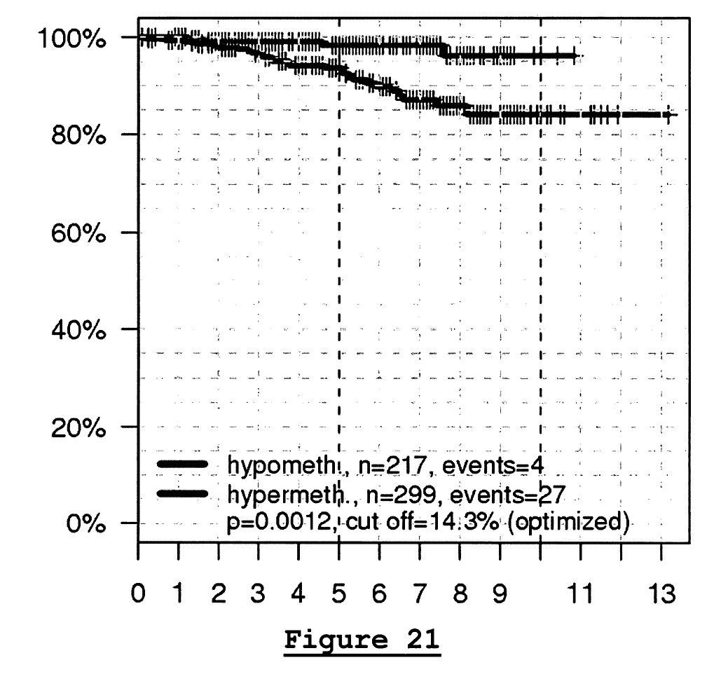

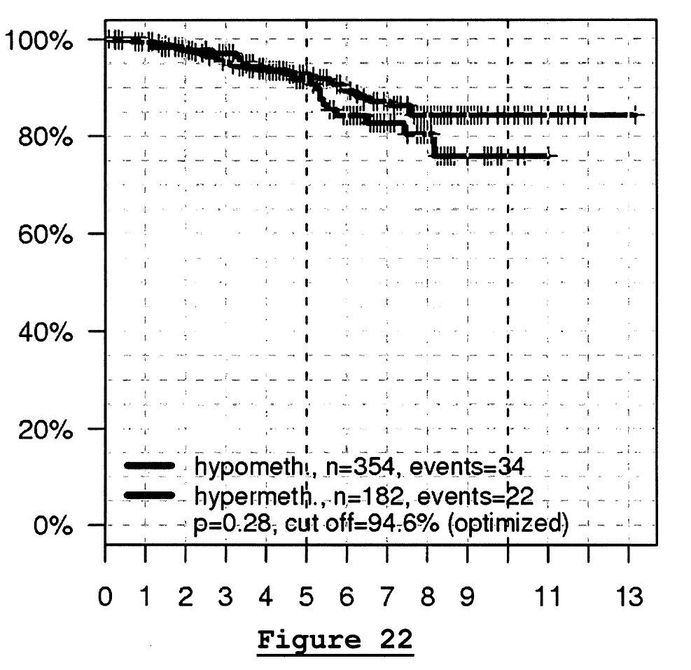

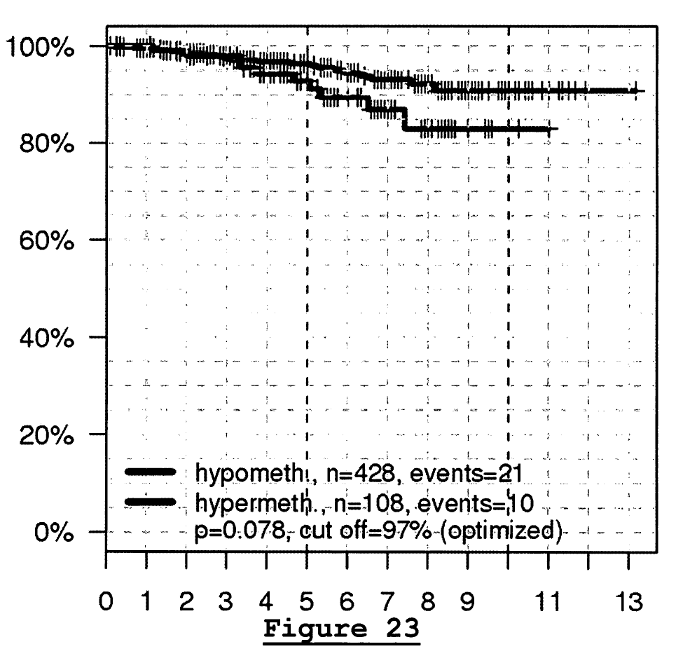

TAM treated (all relapses) ERBB2 (FIG. 14): p-value 0.089; cut off point: 1.3%
TAM treated (distant only) ERBB2 (FIG. 15): p-value 0.084; cut off point: 0.1%
TAM treated (all relapses) TFF1 (FIG. 16): p-value 0.037; cut off point: 50.9%
TAM treated (distant only) TFF1 (FIG. 17): p-value 0.029; cut off point: 52.9%
TAM treated (all relapses) PLAU (FIG. 18): p-value 0.056; cut off point: 4.8%
TAM treated (distant only) PLAU (FIG. 19): p-value 0.065; cut off point: 4.8%
TAM treated (all relapses) PITX2 (FIG. 20): p-value 0.01; cut off point: 13.1%
TAM treated (distant only) PITX2 (FIG. 21): p-value 0.0012; cut off point: 14.3%
TAM treated (all relapses) TBC1D3 (assay II) (FIG. 22): p-value 0.28; cut off point: 94.6%
TAM treated (distant only) TBC1D3 (assay II) (FIG. 23): p-value 0.078; cut off point: 97%

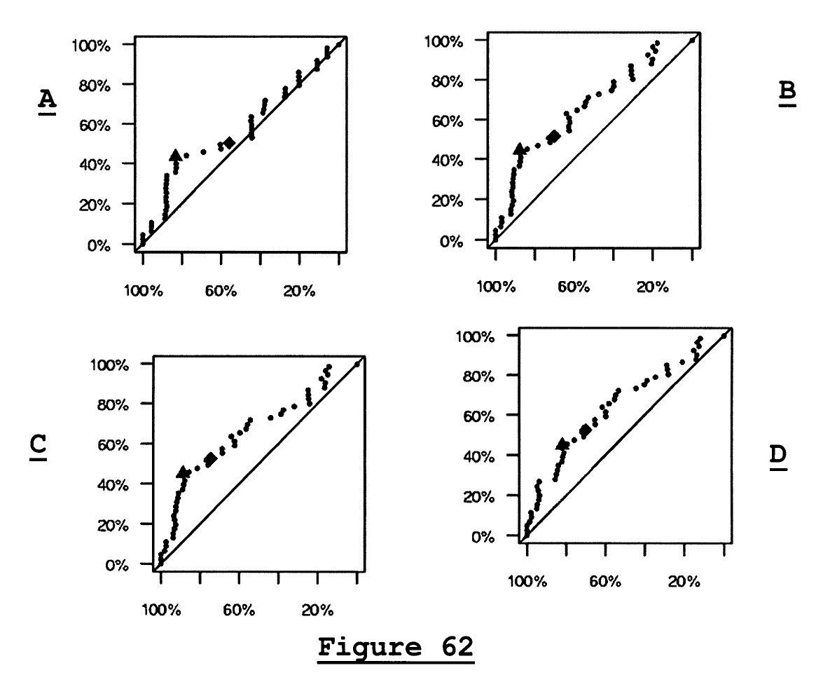

FIG. 62 shows the ROC plot at different times for marker model PITX2 (Assay 1) alone on ER+N0 TAM treated population. Panel A shows the plot at 60 months, panel B shows the plot at 72 months, Panel C shows the plot at 84 months, and Panel D shows the plot at 96 months. Only distant metastasis are defined as events. Sensitivity (proportion of all relapsed patients in poor prognostic group) shown on the X-axis and specificity (proportion of all relapse free patients in good prognostic group) shown on the Y-axis are calculated from KM estimates, and the estimated area under the curve (AUC) is calculated. Values for median cut off (triangle) and best cut off (diamond, 0.42 quantile) are plotted.
AUC 60 months: 0.6
AUC 72 months: 0.69
AUC 84 months: 0.69
AUC 96 months: 0.67

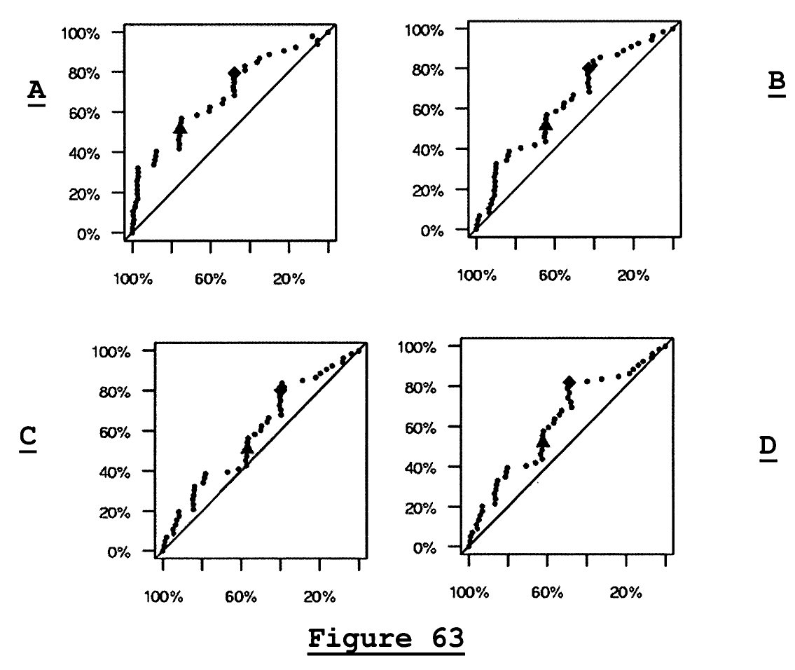

FIG. 63 shows the ROC plot at different times for marker model TFF1 on ER+N0 TAM treated population. Panel A shows the plot at 60 months, panel B shows the plot at 72 months, panel C shows the plot at 84 months and panel D shows the plot at 96 months. Only distant metastasis are defined as events. Sensitivity (proportion of all relapsed patients in poor prognostic group) shown on the X-axis and specificity (proportion of all relapse free patients in good prognostic group) shown on the Y-axis are calculated from KM estimates for different thresholds (=5, 6, 7, 8 years) and the estimated area under the curve (AUC) is calculated. Values for median cut off (triangle) and best cut off (diamond, 0.78 quantile) are plotted.
AUC 60 months: 0.7
AUC 72 months: 0.65
AUC 84 months: 0.61
AUC 96 months: 0.64

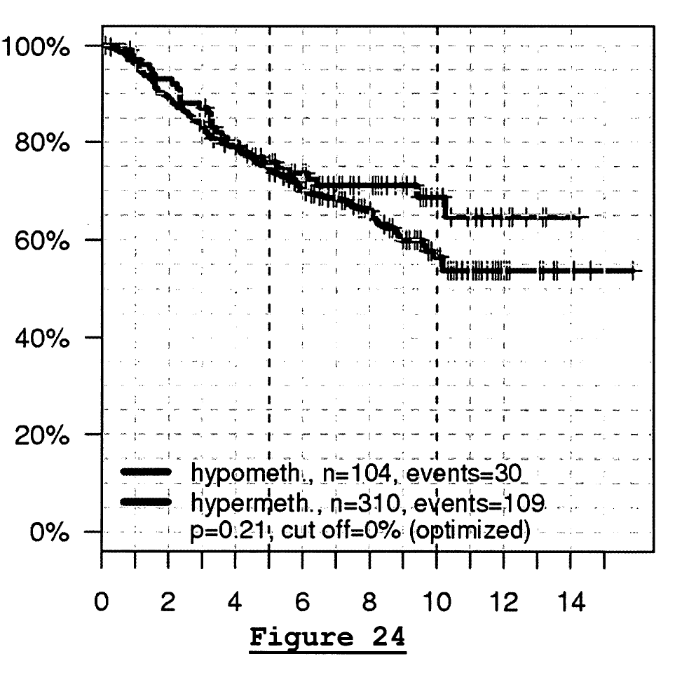

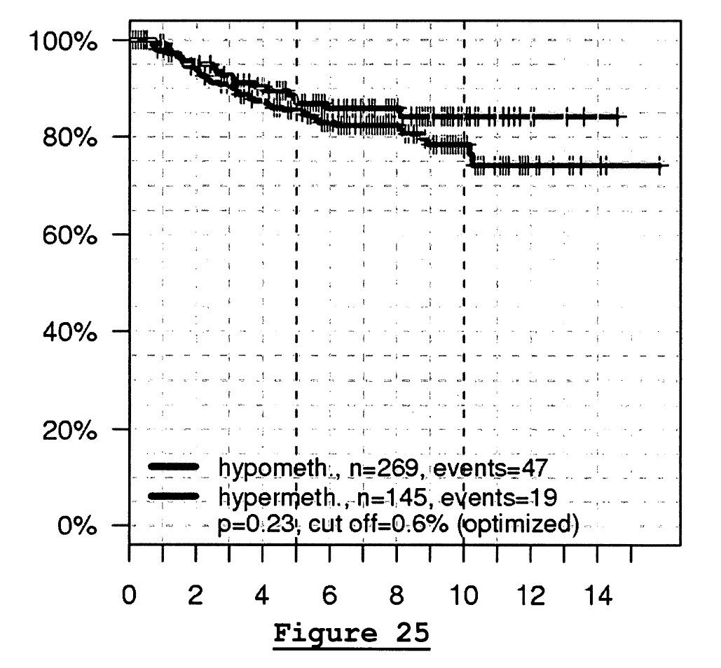

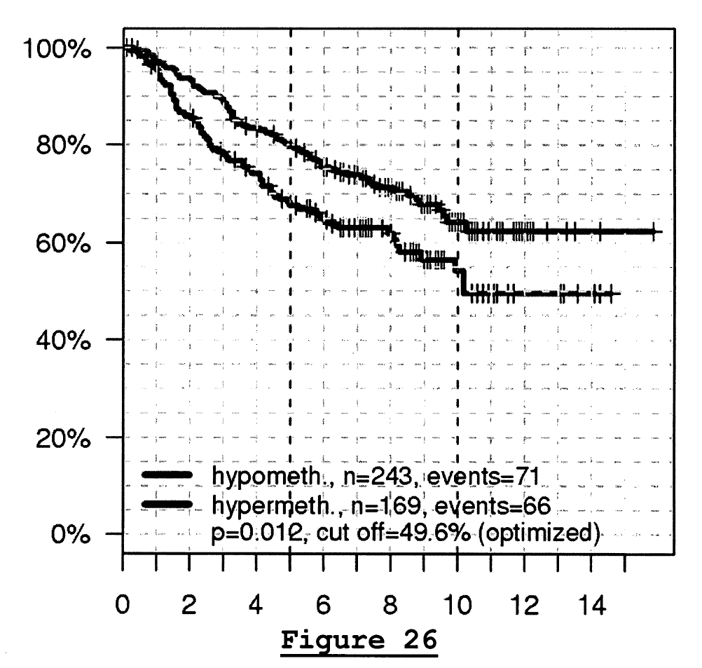

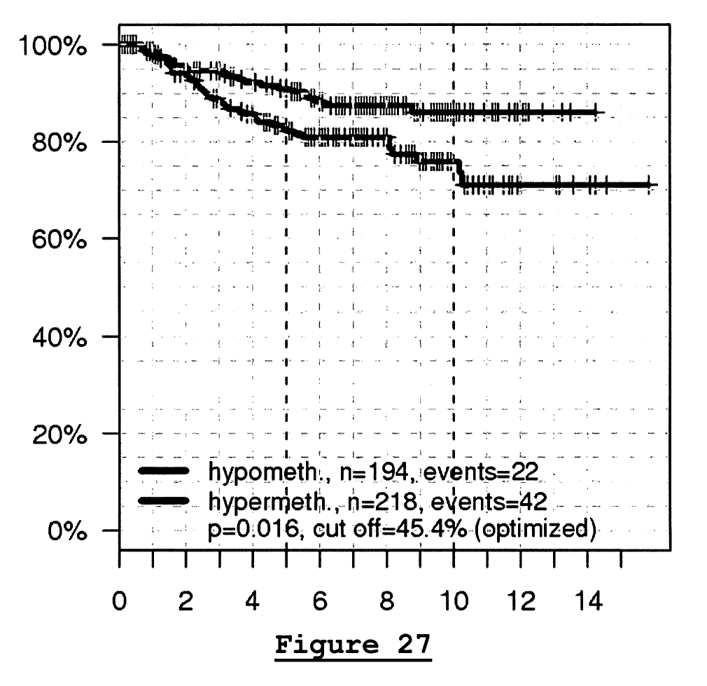

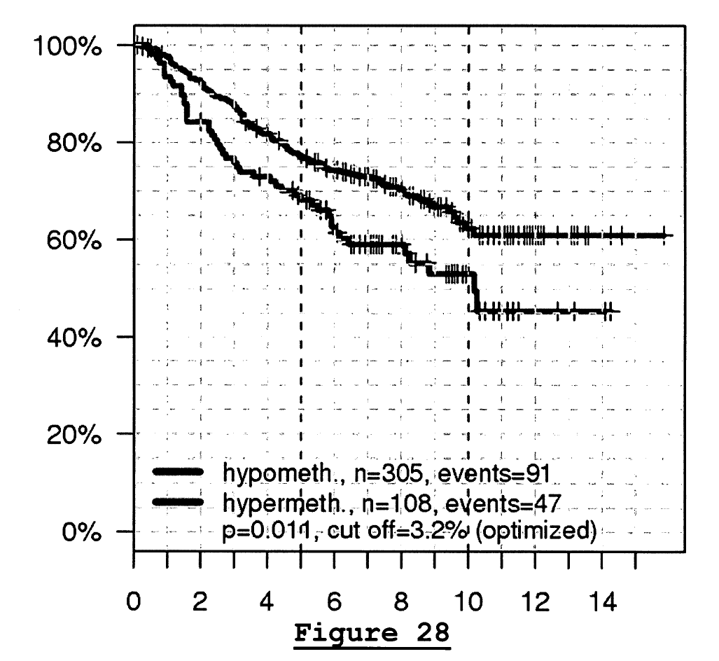

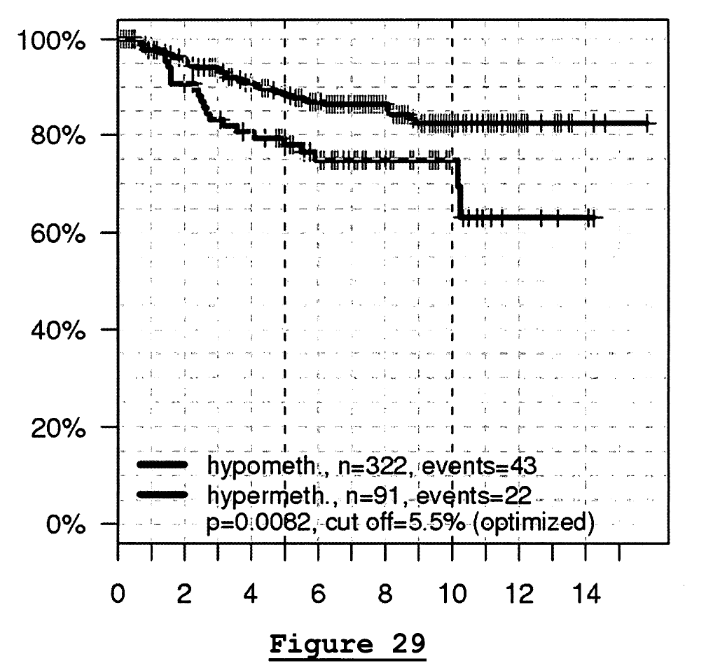

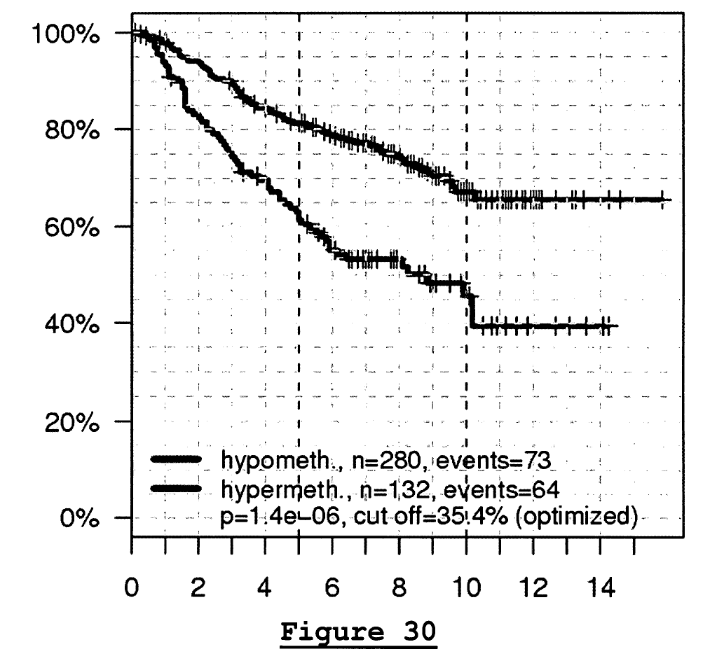

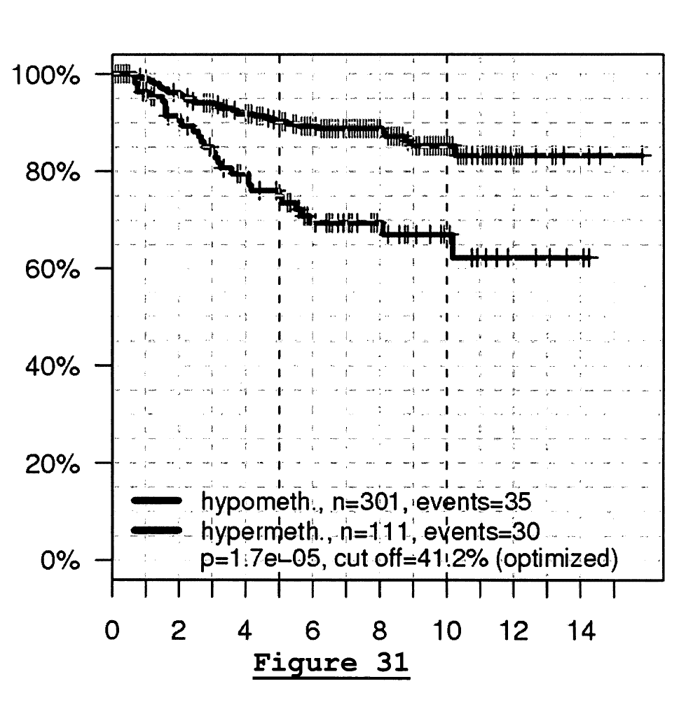

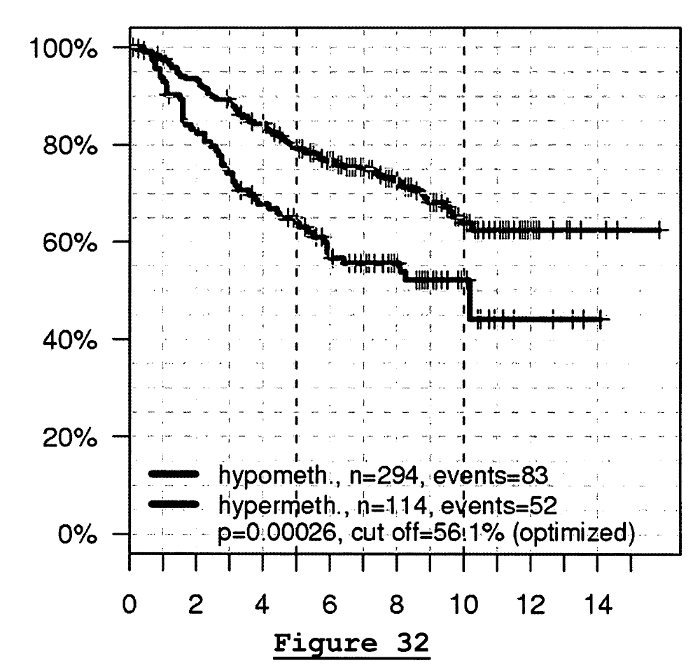

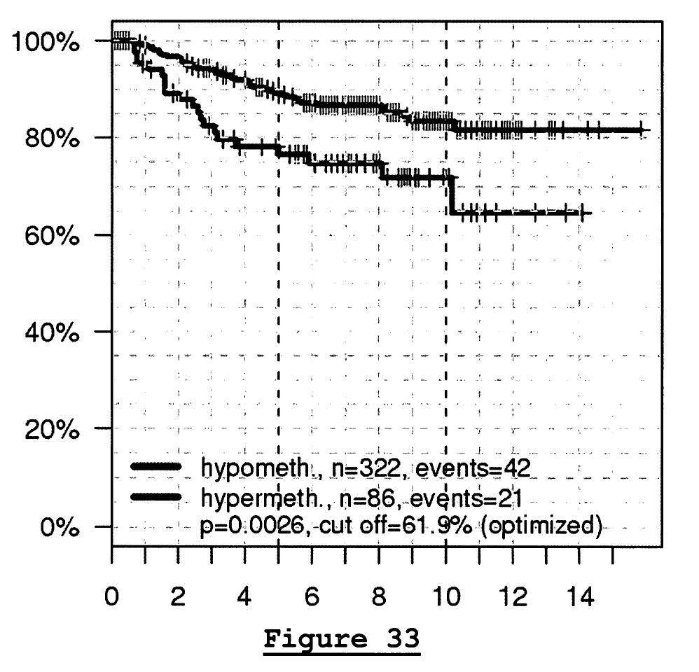

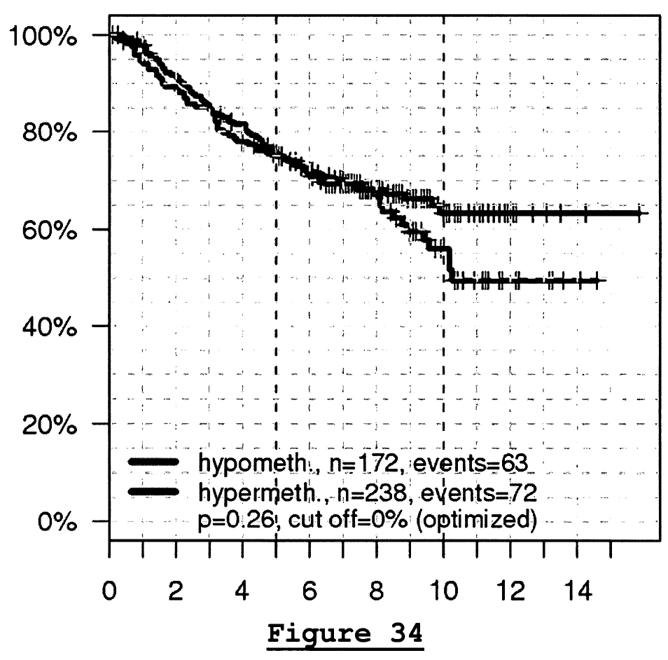

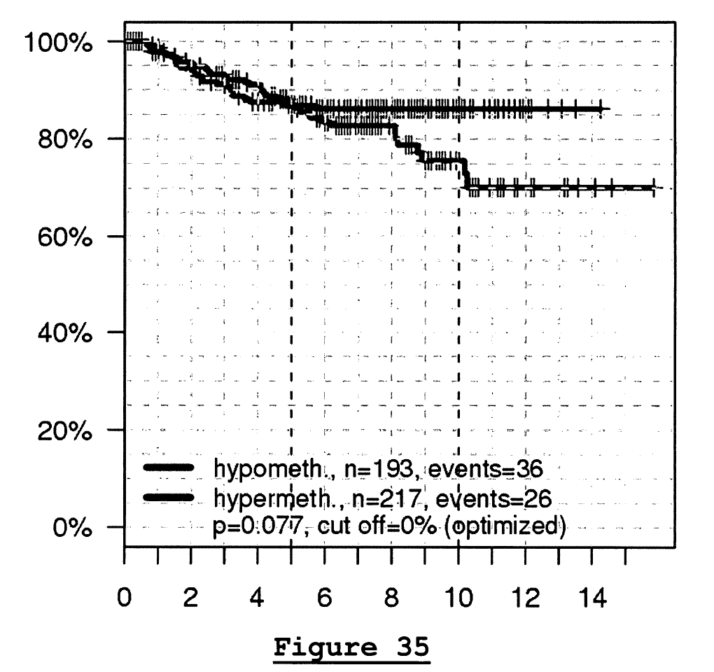

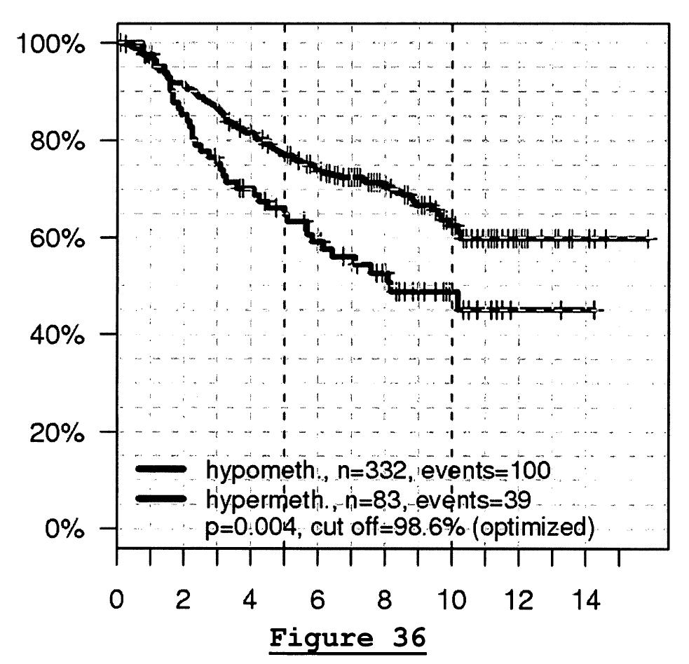

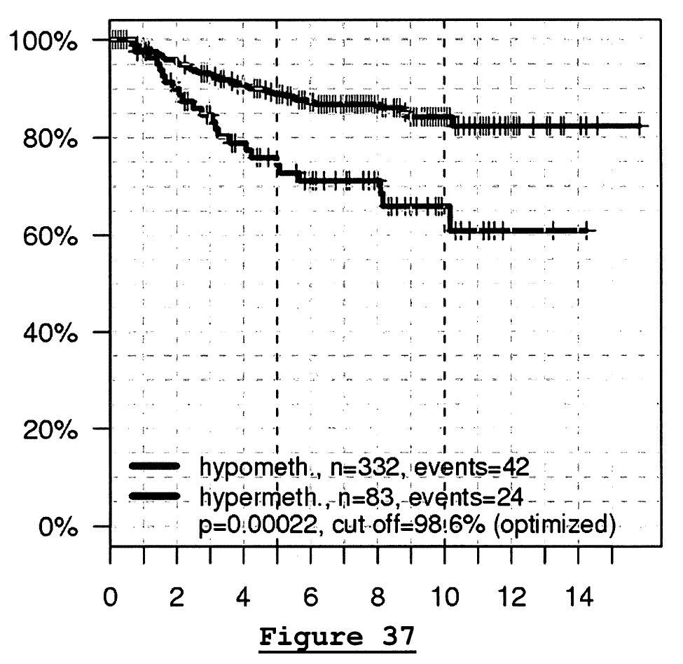

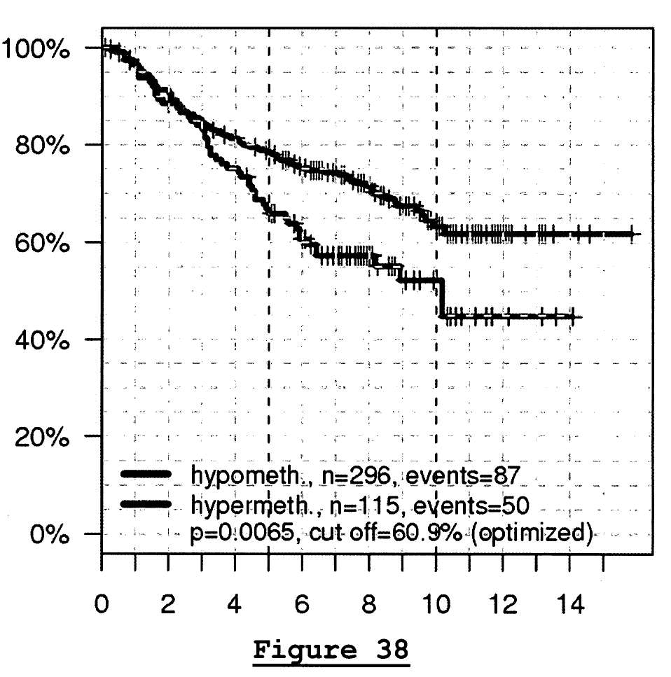

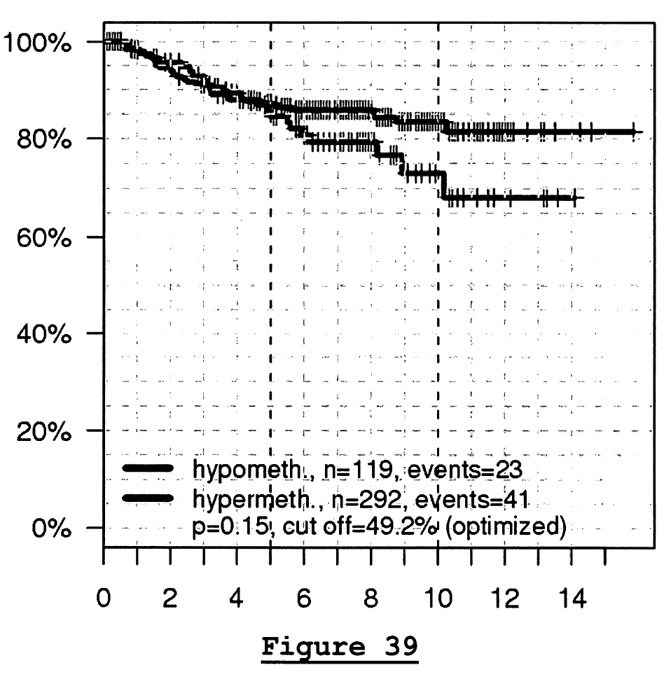

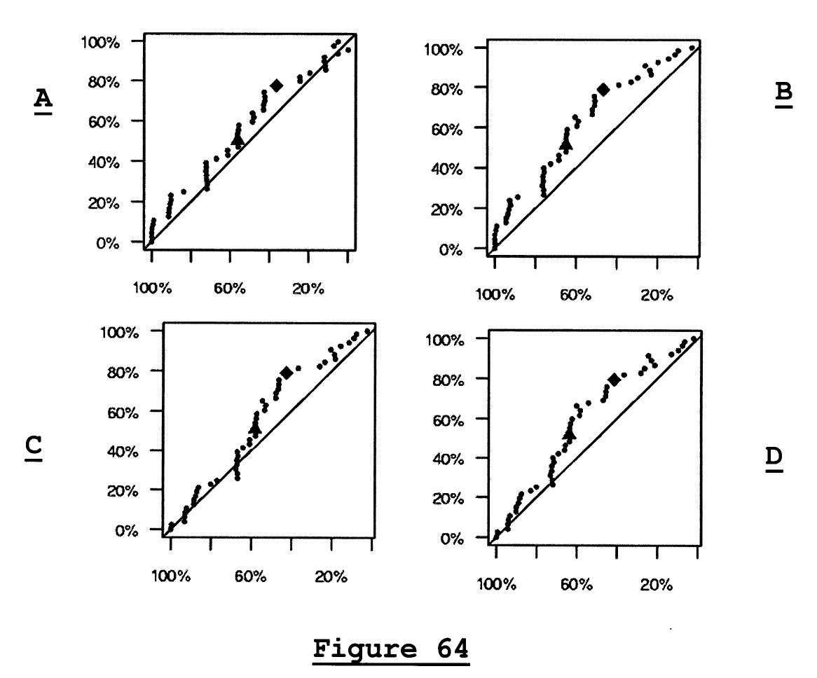

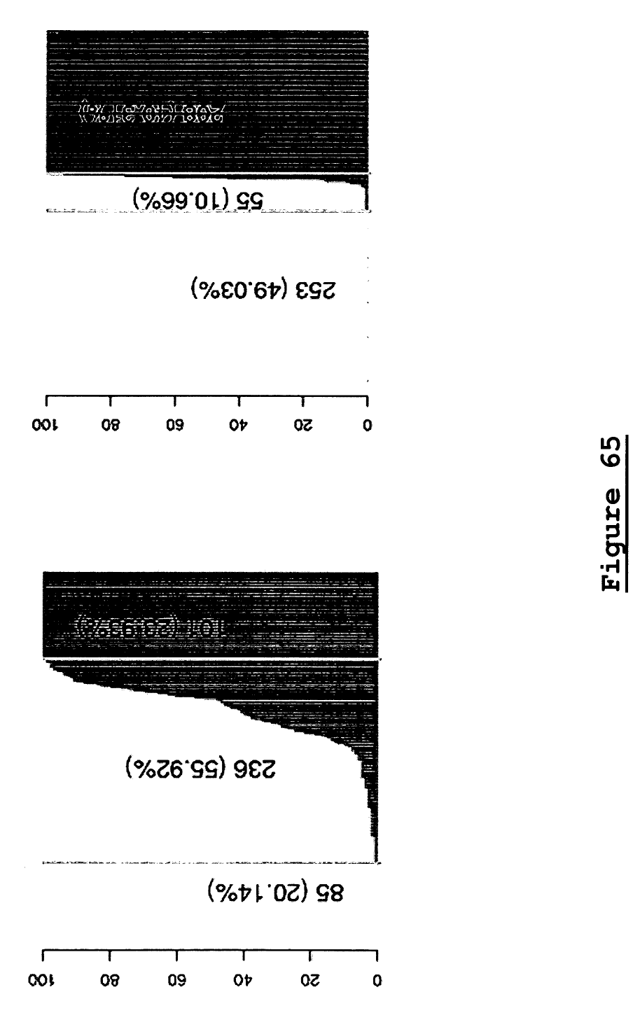

FIG. 64 shows the ROC plot at different times for marker model PLAU on ER+N0 TAM treated population. Panel A shows the plot at 60 months, panel B shows the plot at 72 months, panel C shows the plot at 84 months and panel D shows the plot at 96 months. Only distant metastasis are defined as events. Sensitivity (proportion of all relapsed patients in poor prognostic group) shown on the X-axis and specificity (proportion of all relapse free patients in good prognostic group) shown on the Y-axis are calculated from KM estimates for different thresholds (=5, 6, 7, 8 years), and the estimated area under the curve (AUC) is calculated. Values for median cut off (triangle) and best cut off (diamond, 0.77 quantile) are plotted.
AUC 60 months: 0.6
AUC 72 months: 0.63
AUC 84 months: 0.57
AUC 96 months: 0.6
   Non Tamoxifen Treated
Non Tamoxifen treated (all relapses) ERBB2 (FIG. 24): p-value 0.21; cut off point: 0%;
Non Tamoxifen treated (distant only) ERBB2 (FIG. 25): p-value 0.23; cut off point: 0.6%,
Non Tamoxifen treated (all relapses) TFF1 (FIG. 26): p-value 0.012; cut off point: 49.6%;
Non Tamoxifen treated (distant only) TFF1 (FIG. 27): p-value 0.016; cut off point: 45.4%;
Non Tamoxifen treated (all relapses) PLAU (FIG. 28): p-value 0.011; cut off point: 3.2%;
Non Tamoxifen treated (distant only) PLAU (FIG. 29): p-value 0.0082; cut off point: 5.5%;
Non Tamoxifen treated (all relapses) PITX2 (I) (FIG. 30): p-value 1.4e-06; cut off point: 35.4%;
Non Tamoxifen treated (distant only) PITX2 (I) (FIG. 31): p-value 1.7 e-05; cut off point: 41.2%;
Non Tamoxifen treated (all relapses) PITX2 (II) (FIG. 32): p-value 0.00026; cut off point: 56.1%;
Non Tamoxifen treated (distant only) PITX2 (II) (FIG. 33): p-value 0.0026; cut off point: 61.9%;
Non Tamoxifen treated (all relapses) ONECUT (FIG. 34): p-value 0.26; cut off point: 0%;
Non Tamoxifen treated (distant only) ONECUT (FIG. 35): p-value 0.77; cut off point: 0%;
Non Tamoxifen treated (all relapses) TBC1D3 (FIG. 36): p-value 0.004; cut off point: 98.6%;
Non Tamoxifen treated (distant only) TBC1D3 (FIG. 37): p-value 0.00022; cut off point: 98.6%;
Non Tamoxifen treated (all relapses) ABCA8 (FIG. 38): p-value 0.0065; cut off point: 60.9%;
Non Tamoxifen treated (distant only) ABCA8 (FIG. 39): p-value 0.15; cut off point: 49.2%
   Panels

Based on the results of the single marker evaluations, it was decided to build models using the marker candidates PITX2-Assay I, TFF1, and PLAU. All possible combinations of these markers were evaluated.

Tamoxifen Treated

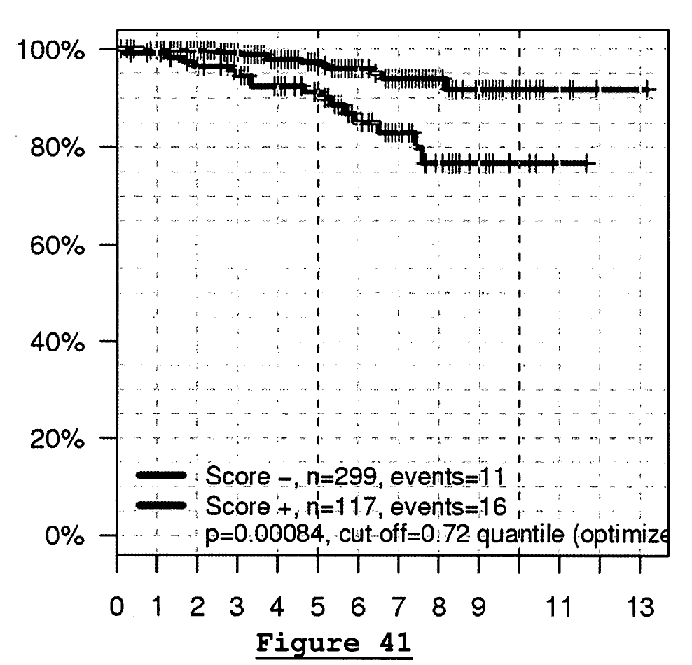

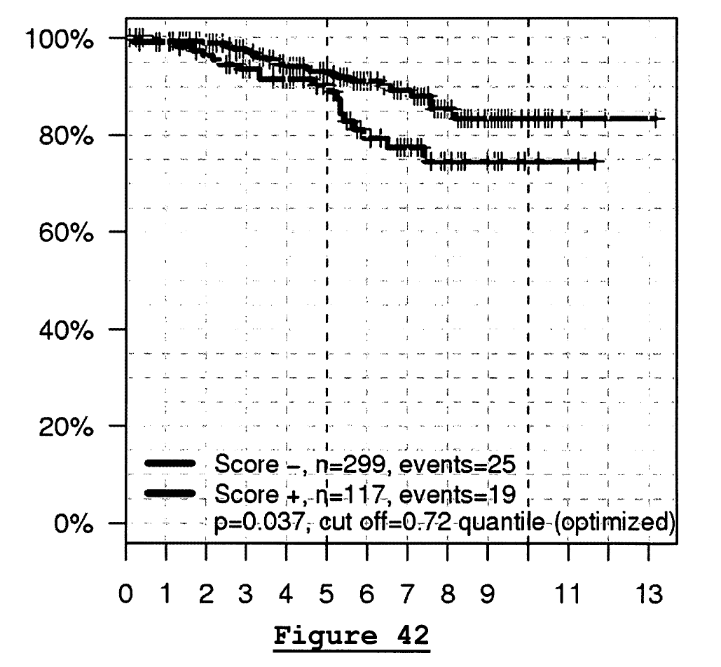

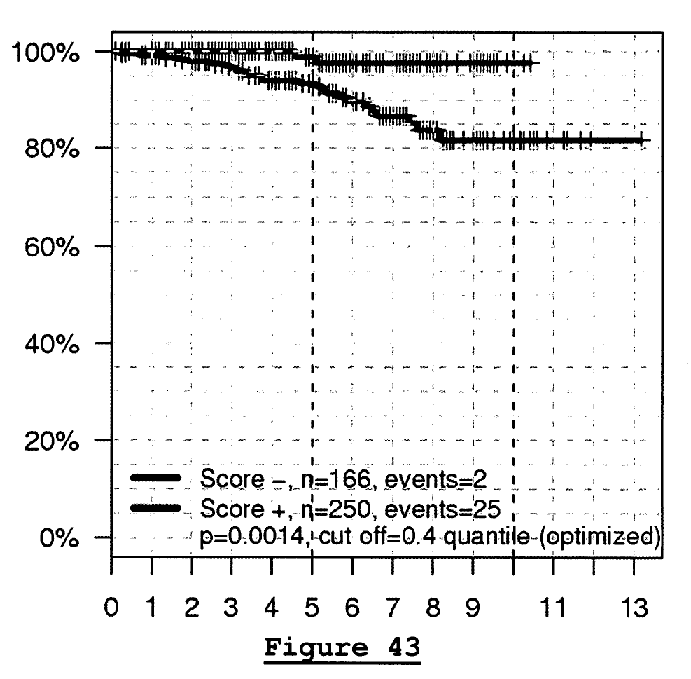

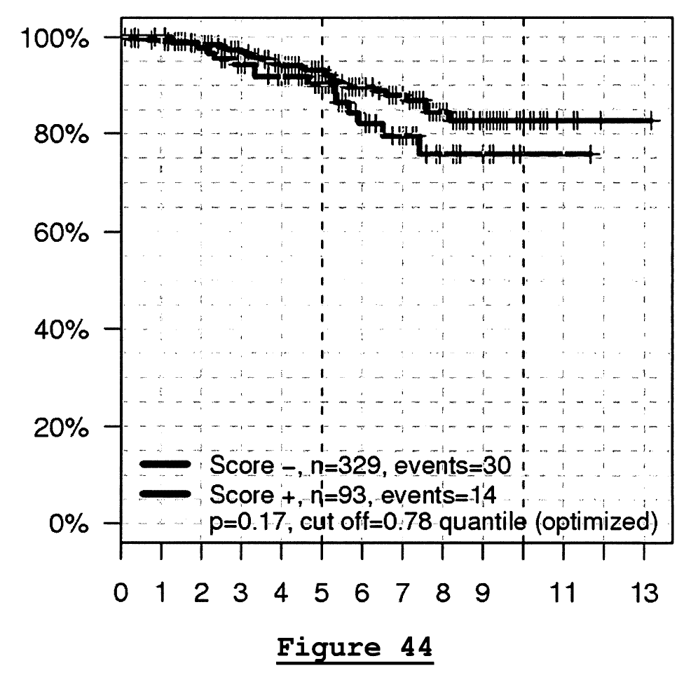

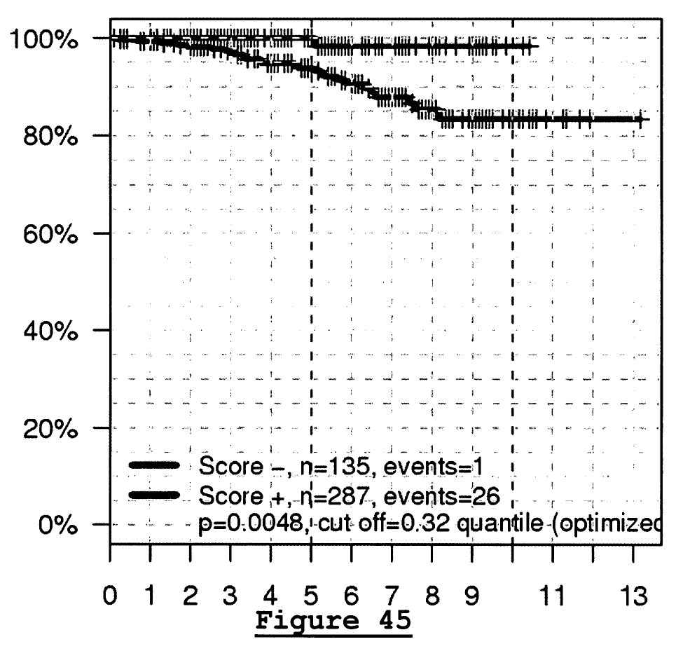

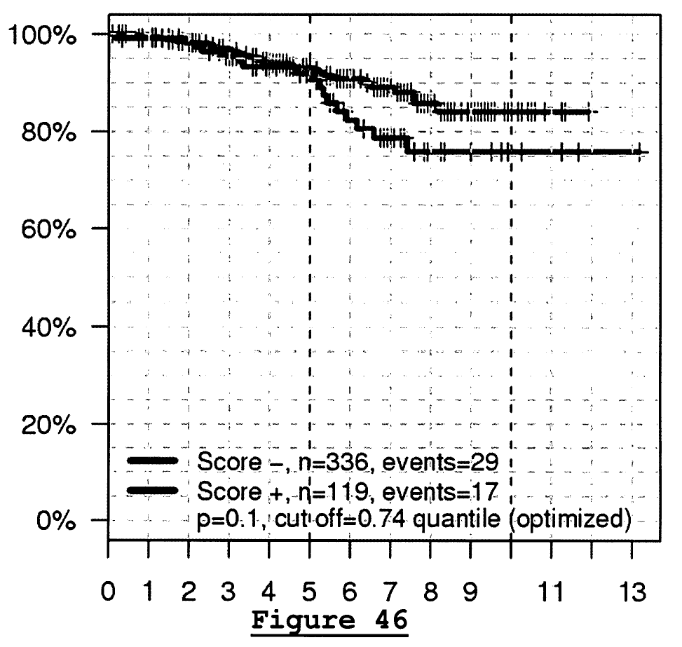

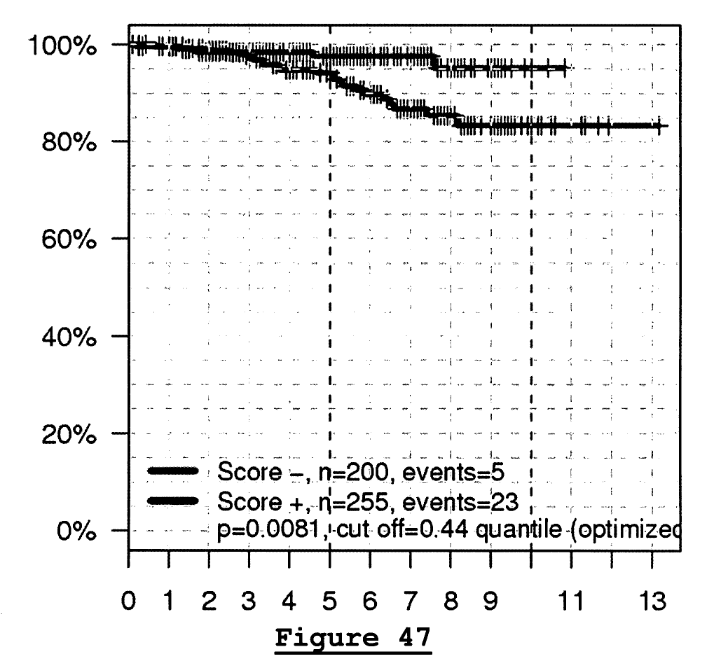

TAM treated (all relapses) TFF1 & PLAU (FIG. 40): p-value 0.023; cut off point: 0.7 quantile;
TAM treated (distant only) TFF1 & PLAU (FIG. 41): p-value 0.00084; cut off point: 0.72 quantile;
TAM treated (all relapses) TFF1 & PLAU & PITX2 (FIG. 42): p-value 0.037; cut off point: 0.72 quantile;
TAM treated (distant only) TFF1 & PLAU & PITX2 (FIG. 43): p-value 0.0014; cut off point: 0.4 quantile;
TAM treated (all relapses) PITX2 & TFF1 (FIG. 44): p-value 0.17; cut off point: 0.78 quantile;
TAM treated (distant only) PITX2 & TFF1 (FIG. 45): p-value 0.0048; cut off point: 0.32 quantile;
TAM treated (all relapses) PITX2 & PLAU (FIG. 46): p-value 0.1; cut off point: 0.74 quantile;
TAM treated (distant only) PITX2 & PLAU (FIG. 47): p-value 0.0081; cut off point: 0.44 quantile.

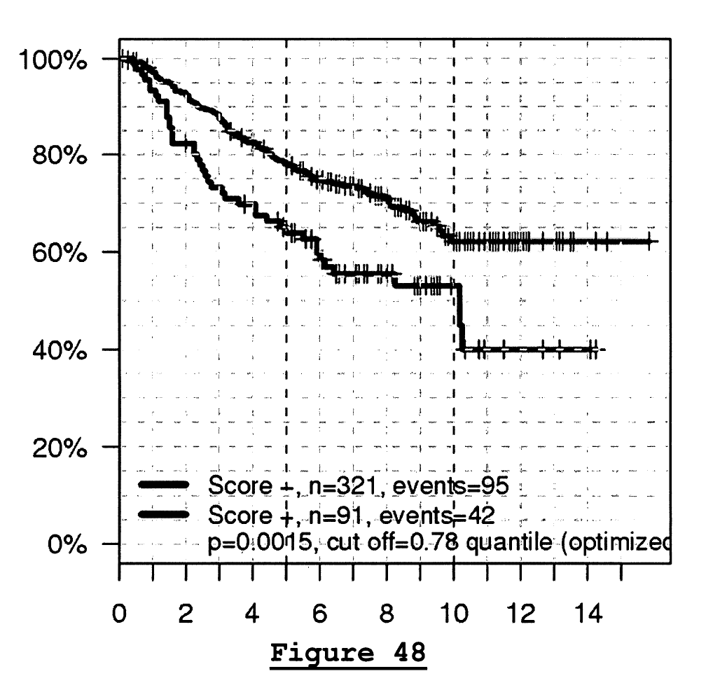

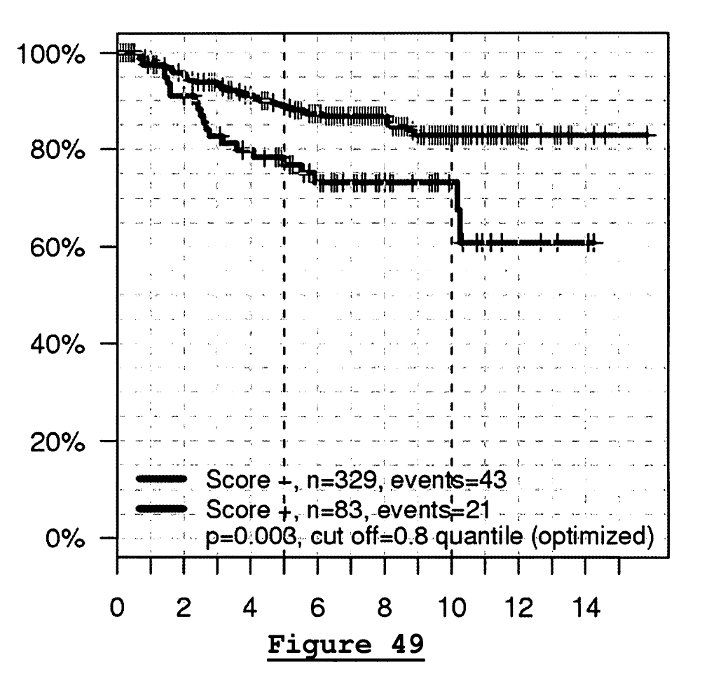

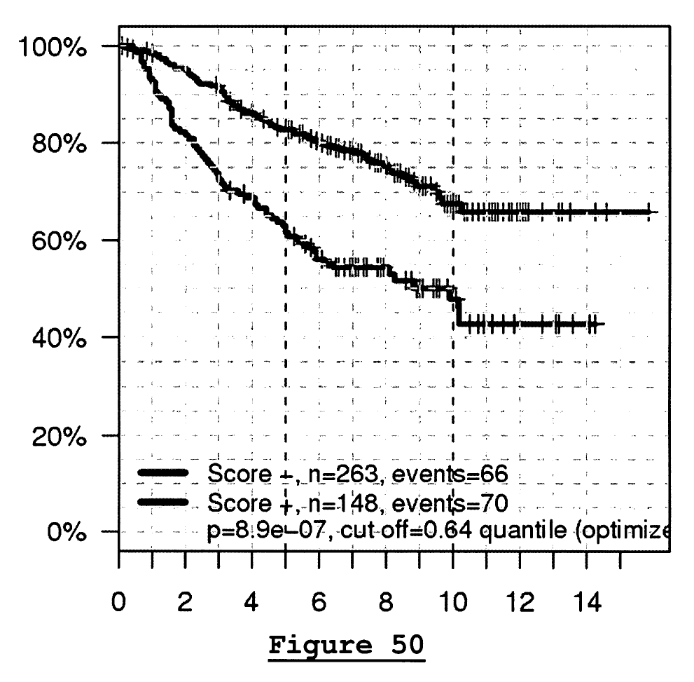

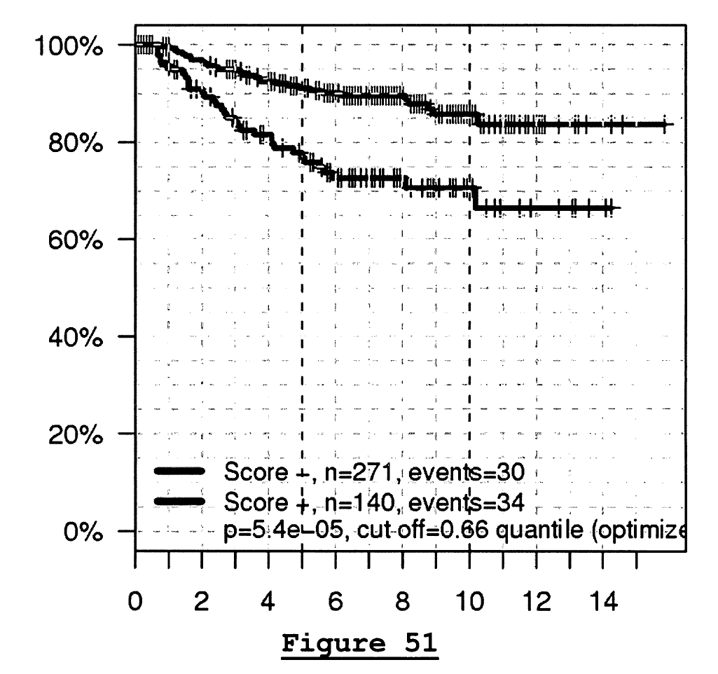

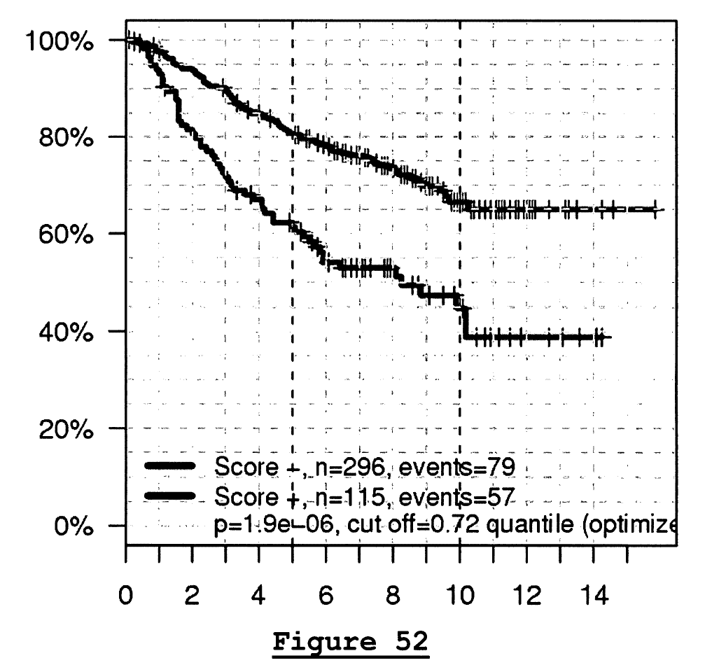

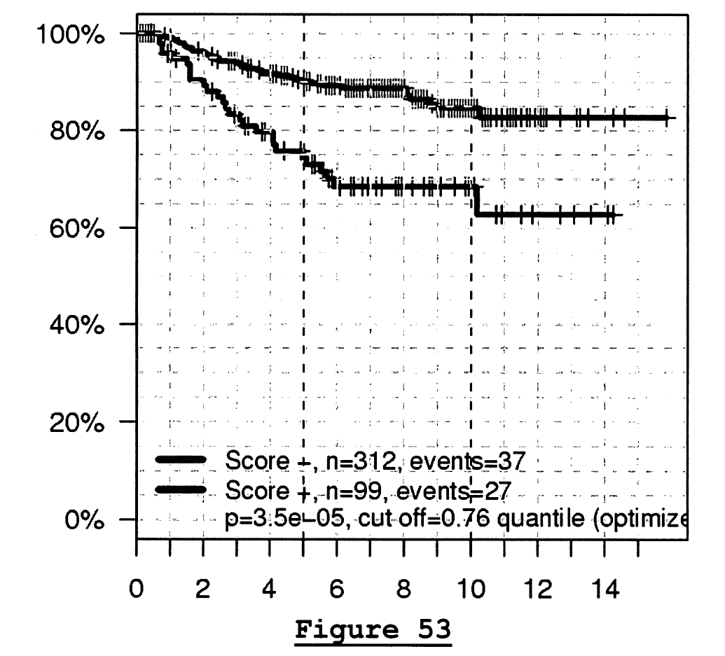

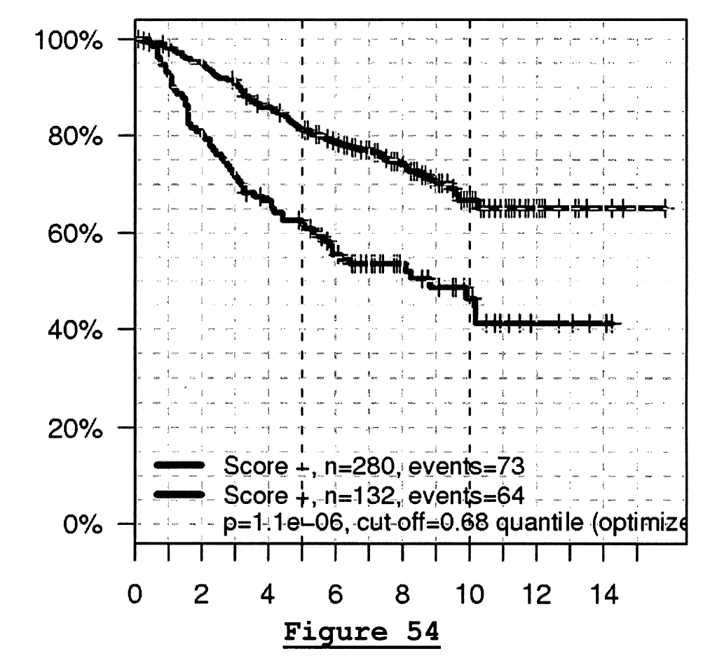

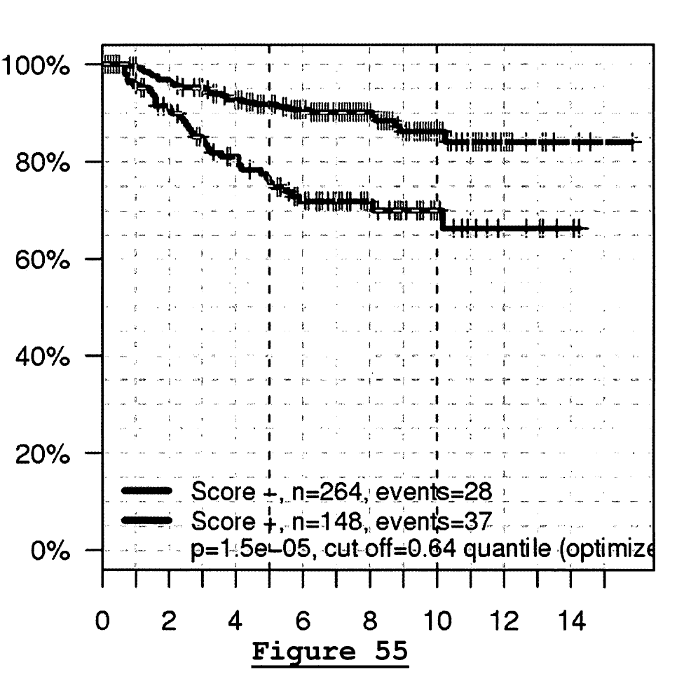

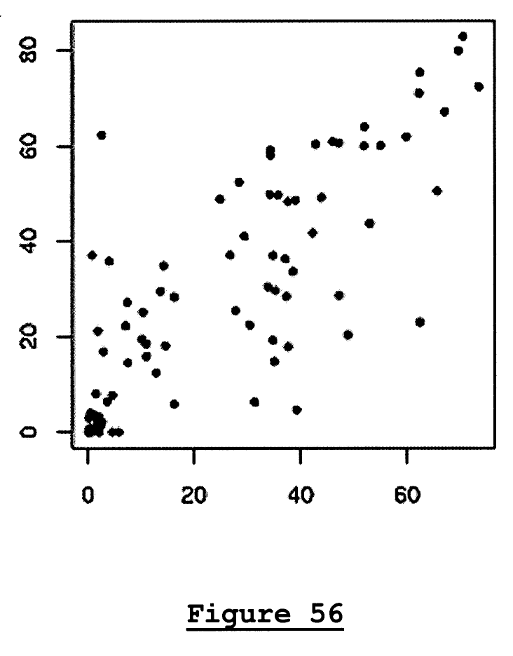

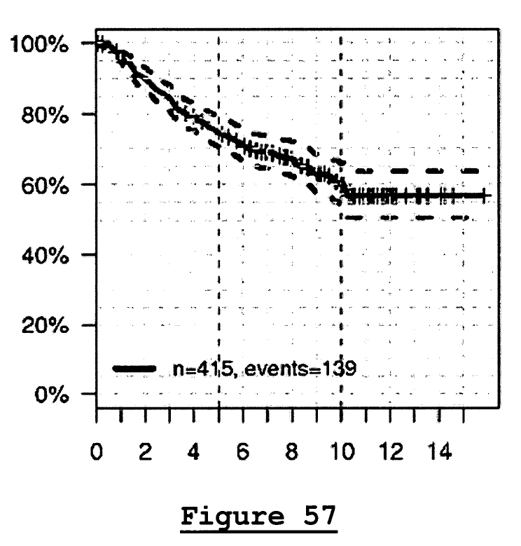

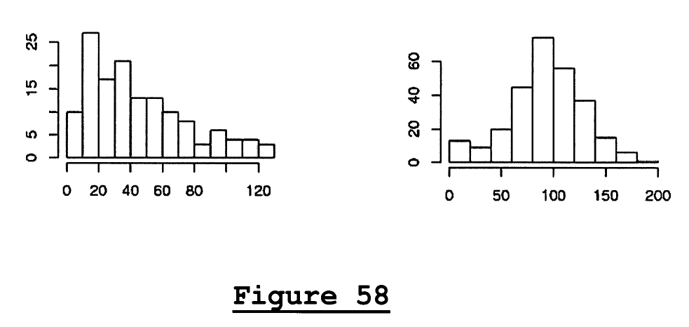

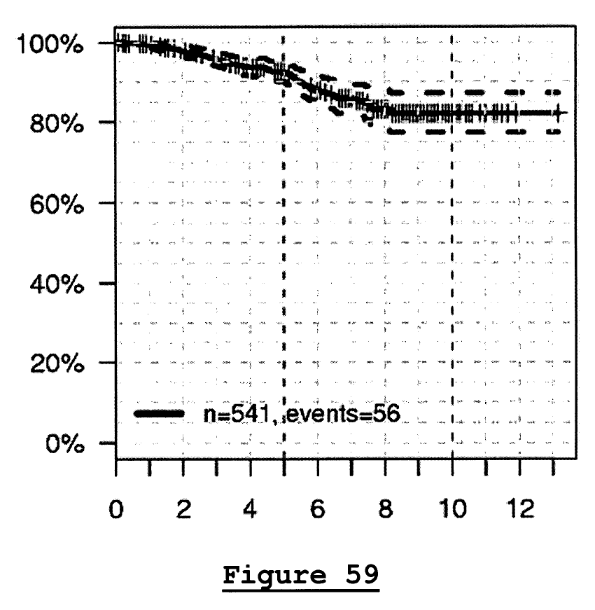

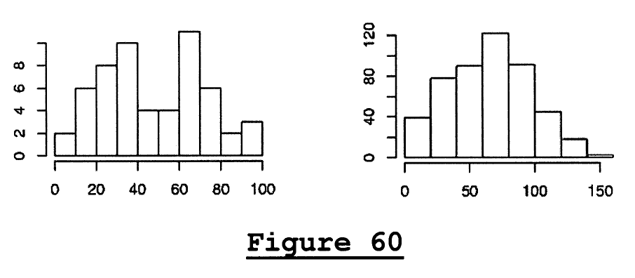

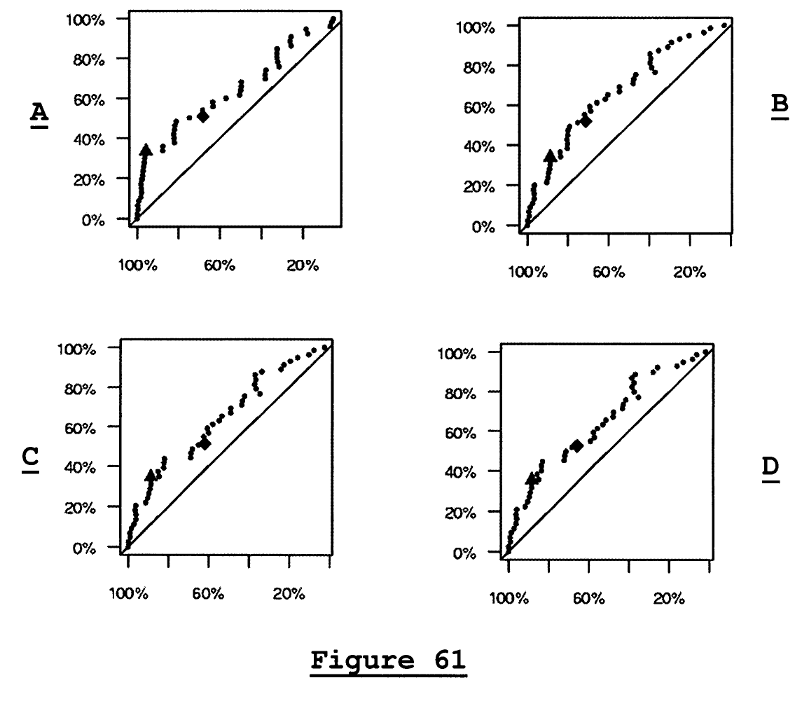

FIG. 61 shows the ROC plot at different times for marker model PITX2 (Assay 1) and TFF1 on ER+N0 TAM treated population. Panel A shows the plot at 60 months, panel B shows the plot at 72 months, panel C shows the plot at 84 months and panel D shows the plot at 96 months. Only distant metastasis are defined as events. Sensitivity (proportion of all relapsed patients in poor prognostic group) shown on the X-axis and specificity (proportion of all relapse free patients in good prognostic group) shown on the Y-axis are calculated from KM estimates, and the estimated area under the curve (AUC) is calculated. Values for median cut off (triangle) and best cut off (diamond, 0.32 quantile) are plotted.
AUC 60 months: 0.62
AUC 72 months: 0.67
AUC 84 months: 0.63
AUC 96 months: 0.65
   Non Tamoxifen Treated
Non Tamoxifen treated (all relapses) TFF1 & PLAU (FIG. 48): p-value 0.0015; cut off point: 0.78 quantile;
Non Tamoxifen treated (distant only) TFF1 & PLAU (FIG. 49): p-value 0.003; cut off point: 0.8 quantile;
Non Tamoxifen treated (all relapses) TFF1 & PLAU & PITX2 (FIG. 50): p-value 8.9e-07; cut off point: 0.64 quantile;
Non Tamoxifen treated (distant only) TFF1 & PLAU & PITX2 (FIG. 51): p-value 5.4e-05; cut off point: 0.66 quantile;
Non Tamoxifen treated (all relapses) PITX2 & TFF1 (FIG. 52): p-value 1.9e-06; cut off point: 0.72 quantile;
Non Tamoxifen treated (distant only) PITX2 & TFF1 (FIG. 53): p-value 3.5e-05; cut off point: 0.76 quantile;
Non Tamoxifen treated (all relapses) PITX2 & PLAU (FIG. 54): p-value 1.1e-06; cut off point: 0.68 quantile;
Non Tamoxifen treated (distant only) PITX2 & PLAU (FIG. 55): p-value 1.5e-05; cut off point: 0.64 quantile.
   Robustness of Marker Models

To evaluate the robustness of the models, a crossvalidation was performed on model marker panel PITX2 (Assay 1) plus TFF1 and marker panel PITX2 (Assay 1) alone, with 200 replicates. The stability of the assignment of one certain patient to the bad or good outcome group is illustrated in FIG. 65, the left hand figure shows model marker panel PITX2 (Assay 1) plus TFF1 and the right hand figure shows model marker panel PITX2 (Assay 1) alone. The plot illustrates in how many crossvalidation replicates each patient get's assigned to group 1 (light gray) or group 2 (dark gray).

TABLE 4
Numbers of censored and relapsed patients in randomly selected
sample set of ER+, N0, untreated population.

| | Frequency | Percentage |
|---|---|---|
| Censored | 276 | 66.5 |
| Distant metastasis | 66 | 15.9 |
| Locoregional relapse | 49 | 11.8 |
| Contralateral breast | 24 | 5.8 |
| Sum | 415 | 100.0 |

TABLE 5
Numbers of censored and relapsed patients
in ER+, N0, TAM treated population.

| | Frequency | Percentage |
|---|---|---|
| Censored | 485 | 89.6 |
| Distant metastasis | 31 | 5.7 |
| Locoregional relapse | 20 | 3.7 |
| Contralateral breast | 5 | 0.9 |
| Sum | 541 | 100.0 |

1. A method for providing at least one of a prognosis for, and predicting the outcome of endocrine treatment of a subject with a cell proliferative disorder of the breast tissue, comprising:
a) obtaining a biological sample from a subject; and
b) determining, within the sample, expression of at least one of the PITX2 gene, and the regulatory sequences thereof; whereby at least one of a prognosis for, and predicting the outcome of endocrine treatment of the subject is, at least in part, afforded.

2. The method of claim 1, further comprising in b), determining expression of at least one of the TFF1 gene, the PLAU genes, and the regulatory sequences thereof.

3. The method of claim 1, further comprising in b), determining expression of at least one of the PLAU gene, and the regulatory sequences thereof.

4. The method of claim 1, further comprising in b), determining expression of at least one of the TFF1 gene, and the regulatory sequences thereof.

5. The method of any one of claims 1 to 4, wherein the subject is estrogen receptor positive.

6. The method of any one of claims 1 to 4, further comprising:
d) determining a suitable treatment regimen for the subject.

7. The method of claim 6, wherein the suitable treatment regimen comprises one or more therapies selected from the group consisting of chemotherapy, radiotherapy, surgery, biological therapy, immunotherapy, antibodies, molecularly targeted drugs, estrogen receptor modulators, estrogen receptor down-regulators, aromatase inhibitors, ovarian ablation, LHRH analogues and other centrally acting drugs influencing estrogen production.

8. The method of any one of claims 1 to 4, wherein the cell proliferative disorder of the breast tissue is selected from the group consisting of ductal carcinoma in situ, invasive ductal carcinoma, invasive lobular carcinoma, lobular carcinoma in situ, comedocarcinoma, inflammatory carcinoma, mucinous carcinoma, scirrhous carcinoma, colloid carcinoma, tubular carcinoma, medullary carcinoma, metaplastic carcinoma, and papillary carcinoma and papillary carcinoma in situ, undifferentiated or anaplastic carcinoma and Paget's disease of the breast, and combinations thereof.

9. The method of any one of claims 1 to 4, wherein expression is determined by analysis of at least one of mRNA expression, LOH, and protein expression.

10. The method of any one of claims 1 to 4, wherein expression is determined by analysis of the methylation status of one or more CpG positions within the genes or regulatory regions thereof.

11. A method for providing at least one of a prognosis for, and predicting the outcome of endocrine treatment of a subject with a cell proliferative disorder of the breast tissue, comprising:
a) isolating genomic DNA from a biological sample obtained from a subject;
b) treating the genomic DNA, or a fragment or portion thereof, with one or more reagents suitable to convert 5-position unmethylated cytosine bases to uracil or to another base that is detectably dissimilar to cytosine in terms of hybridization properties;
c) contacting the treated genomic DNA, or the treated fragment or portion thereof, with an amplification enzyme and at least two primers comprising, in each case a contiguous sequence at least 18 nucleotides in length that is complementary to, or hybridizes under moderately stringent or stringent conditions to a sequence selected from the group consisting of SEQ ID NOS:150, 151, 155, 156, and complements thereof, wherein the treated DNA, or the fragment or portion thereof is either amplified to produce one or more amplificates, or is not amplified;
d) determining, based on the presence or absence of, or on the quantity or on a property of said amplificate, the methylation state of at least one CpG dinucleotide sequence of SEQ ID NO:149, or an average, or a value reflecting an average methylation state of a plurality of CpG dinucleotide sequences of SEQ ID NO: 149, whereby at least one of a prognosis for, and predicting the outcome of endocrine treatment of the subject is, at least in part, afforded.

12. The method of claim 11, further comprising contacting in c) with at least two primers comprising, in each case a contiguous sequence at least 18 nucleotides in length that is complementary to, or hybridizes under moderately stringent or stringent conditions to a sequence selected from the group consisting of SEQ ID NOS:76-103, SEQ ID NOS:153, 154, 157 and 158, and complements thereof.

13. The method of any one of claims 11 or 12, wherein in b), the one or more reagents comprises a solution selected from the group consisting of bisulfite, hydrogen sulfite, disulfite, and combinations thereof.

14. The method of any one of claims 11 and 12, wherein determining in d) comprises one or more methods taken from the group consisting of oligonucleotide hybridization analysis, Ms-SnuPE, sequencing, Real-Time detection probes, and oligonucleotide array analysis.

15. A nucleic acid molecule consisting of a sequence at least 18 bases in length of a contiguous sequence selected from the group consisting of SEQ ID NOS:2-5, SEQ ID NOS:151-158, and SEQ ID NOS:76-103, and contiguous portions thereof.

16. An oligomer consisting essentially of at least one base sequence having a length of at least 10 contiguous nucleotides, which hybridizes to or is identical to one of the nucleic acid sequences selected from the group consisting of SEQ ID NOS:2-5, SEQ ID NOS:151-158, and SEQ ID NOS:76 to SEQ ID NO:103.

17. The oligomer of claim 16, wherein the oligomer is an oligonucleotide or a peptide nucleic acid (PNA)-oligomer.

18. A composition, comprising:
a nucleic acid comprising a sequence at least 18 contiguous bases in length of a chemically pretreated genomic DNA sequence selected from the group comprising of SEQ ID NOS:2-5, SEQ ID NOS:151, 152, 155, 156, sequences complementary thereto, and contiguous portions thereof; and
a buffer, comprising at least one of: magnesium chloride, dNTP, taq polymerase, an oligomer comprising at least one base sequence having a length of at least 9 contigous nucleotides that is complementary to, or hybridizes under moderately stringent or stringent conditions to a pre-treated genomic DNA selected from the group consisting of SEQ ID NOS:2-5, SEQ ID NOS:151, 152, 155, 156, sequences complementary thereto, and contiguous portions thereof.

19. A method for providing at least one of a prognosis for, and predicting the outcome of endocrine treatment of a subject with a cell proliferative disorder of the breast tissue, comprising: use, in a suitable methylation assay, of at least one of a nucleic acid according to claim 15, an oligomer according to claim 16, and a composition according to claim 18, whereby at least one of a prognosis for, and predicting the outcome of endocrine treatment of the subject is, at least in part, afforded.
